(19) United States
(12) Patent Application Publication
(10) Pub. No.: US 2026/0090799 A1
(43) Pub. Date: Apr. 2, 2026

(54) Multi-Strand Knotless Tensionable Fixation System and Related Methods

(71) Applicant: Dunamis Medical Technologies, LLC, Greenville, AL (US)

(72) Inventors: Prithviraj Chavan, Greenville, AL (US); Forrest Samuel, Carlsbad, CA (US)

(21) Appl. No.: 19/347,671

(22) Filed: Oct. 1, 2025

Related U.S. Application Data
(60) Provisional application No. 63/702,084, filed on Oct. 1, 2024.
(60) Provisional application No. 63/708,963, filed on Oct. 18, 2024.

(51) Int. Cl. A61B 17/04; A61B 17/00

(52) U.S. Cl. CPC A61B 17/0487; A61B 2017/00477; A61B 2017/0496

(57) ABSTRACT

A knotless tensionable fixation system including a base, a locking element, and a lock screw. The system enables a user to achieve complex soft tissue-to-bone or bone-to-bone repair by first placing the base against a first bone segment (or fixation plate or prosthesis) and then provisionally securing multiple tensionable fixation members that may be attached to the same or different tissue or bone to be repaired. Once all tensionable fixation members have been provisionally secured to the base under the desired tension to achieve reduction of the repair, the locking element may be coupled to the base and secured by the lock screw under compression to provide a secondary or final lock simultaneously to all tensionable fixation members to securely lock all tensionable fixation members under tension at the same time and with one single locking element.

### CROSS-REFERENCES TO RELATED APPLICATIONS

The present application is a non-provisional application claiming the benefit of priority under 35 U.S. C. § 119(e) from U.S. Provisional Application No. 63/702,084, filed on Oct. 1, 2024, and U.S. Provisional Application No. 63/708,963, filed on Oct. 18, 2024, the complete disclosures of which are hereby incorporated by reference into this disclosure as if set forth fully herein.

### FIELD

The present disclosure relates generally to bone fracture and soft tissue repair, and more specifically to an apparatus and method for repairing soft tissue and bone fracture injuries using multiple strands of tension members secured in multiple locations with a single locking element.

### BACKGROUND

Fractures especially in the proximal shoulder area have tendons attached to multiple pieces of bone. During a shoulder fracture fixation procedure, for example, the surgeons sometimes may end up using many sutures to suture through the tendons and the bones to align them followed by a complex process of knot-tying to prevent further displacement of the fracture. The process is tedious from the standpoint of suture management and operation time, as well as the surgeon's ability to follow a certain technique which may optimize a reduction of the fracture while the fracture goes through the process of healing. If adequate reduction cannot be maintained it can result in failure of the repair, non-union, malunion, loss of function and even revision surgery.

### SUMMARY

Given the frequency of these fractures, whether they are treated with plate and screws or joint replacements and the importance of achieving good reduction, healing for optimal patient outcomes, a need exists for a system that can be positioned on the bone, plate, or prosthesis and provide a quick method of passing tensionable fixation members (e.g., surgical sutures) through the system provisionally fixing it using a design feature from the system followed by a secondary mechanism which can augment this fixation. This system will allow for soft tissue to bone and bone to bone healing. The system can also have features which can allow for fixation with wires, guide pin, or screws if required. It brings sutures from multiple different directions and secures them on the system after manually applying adequate tension to optimize reduction in securing the suture on the system. Though this system can be used for any indication requiring suture management and security, it can be extremely helpful in indications like proximal humerus injuries where a significant number of sutures are used and fixed in multiple different directions followed by knot tying to assist with providing adequate repair.

In some embodiments, the multi-strand knotless tensionable fixation system (also referred to herein as “system”) includes a base, a locking element, a lock screw and at least one tensionable fixation member. The system of the present disclosure enables a user to achieve complex soft tissue-to-bone or bone-to-bone repair by first placing the base against a first bone segment (or securing to a fixation plate or prosthesis) and then securing multiple tensionable fixation members (e.g., surgical sutures, tape, wires, etc.) that may be attached to the same or different tissue or bone to be repaired (e.g., several unique tensionable fixation member strands may be attached the same tissue, or unique tensionable fixation member strands may be attached to different bone fragments or tissue segments), or to another artificial fixation member such as a prosthesis. In some embodiments, each tensionable fixation member has one or more free ends and a target engagement portion, the target engagement portion may be attached to a tissue or bone segment to be repaired or a surgical implant or prosthesis, and the tensionable fixation member is passed through the base such that the one or more free ends extend proximally from the base to enable manipulation by a user. In some embodiments, the target engagement portion comprises one end of the tensionable fixation member, which then has one free end. In some embodiments, the target engagement portion may be a loop or middle portion of the tensionable fixation member, which then has two free ends that may be secured by the base. In some embodiments, the system enables each tensionable fixation member to be provisionally secured to the base under adequate tension while other tensionable fixation members (or another strand of the same tensionable fixation member) are being utilized. In some embodiments, once all tensionable fixation members have been provisionally secured to the base under the desired tension to achieve reduction of the repair, the locking element may be coupled to the base and secured by the lock screw under compression to provide a secondary or final lock (e.g., the provisional lock being a first lock) simultaneously to all tensionable fixation members to securely lock all tensionable fixation members under tension at the same time and with one single locking element.

In some embodiments, the base may have any perimeter shape suitable to allow fixation of multiple tensionable fixation members in multiple directions, including but not limited to circular, semi-circular, curved, triangular, square, oval, hexagonal, octagonal, pentagonal, and the like. In some embodiments, the base may have a top or proximal surface, a bottom or distal surface, and a lateral surface, which may be a planar surface or a curved circumferential surface as shown, depending on the perimeter shape of the base. In some embodiments, the base may have a perimeter ridge or lip configured to engage with the perimeter recess of the locking element when the locking element is advanced onto the base. In some embodiments, the perimeter ridge of the base and perimeter recess of the locking element cooperate to form a “locking interface” configured to capture and compress the tensionable fixation members therebetween upon actuation of the lock screw, thereby providing the secondary locking feature.

In some embodiments, the base includes central opening extending through the base from the top surface to the bottom surface along a central axis. In some embodiments, the central opening includes a threaded lumen configured to threadedly receive the threaded post of the lock screw to secure the lock screw to the base. In some embodiments, the base further comprises a plurality of provisional locking cleats (hereinafter “cleats”) distributed around the central opening. In some embodiments, the plurality of cleats may be positioned equidistant from one another in an annular fashion, however other configurations are possible. For example, in some embodiments, the plurality of cleats may be grouped or bunched to one or more sides of the base. In some embodiments, each cleat comprises a round opening formed in the top surface and a tapered extension. In some embodiments, the cleats may be oriented such that the round openings are positioned near the central opening and the tapered extensions extend toward the perimeter ridge. In some embodiments, the tapered extensions are wider near the round opening and narrower near the perimeter ridge. In some embodiments, the top surface may include a plurality of sloped recesses positioned at the radial ends of the tapered extensions and configured to enable smooth passage of the tensionable fixation member from the base.

In some embodiments, the base includes a plurality of lateral openings formed in the lateral surface with each lateral opening providing access to a fixation channel extending diagonally (toward the top surface) into the base. In some embodiments, each lateral opening is aligned with a unique cleat such that the associated fixation channel extends diagonally between the lateral opening and the round opening of the cleat, with the tapered extension being open to the fixation channel. Thus, the base is configured such that, after securing a fixation end (or portion) of a tensionable fixation member to a tissue or bone segment to be repaired, the free end(s) of the tensionable fixation member may be passed (e.g., using a chaperone element such as a needle, passing loop, or guide wire, etc.) into the base through the lateral opening, along the diagonal fixation channel, and emerging through the top surface through the associated cleat, either by way of the round opening or a wider portion of the tapered extension. After pulling on the free end to apply a desired amount of tension to the tensionable fixation member, the user may then pull laterally on the tensionable fixation member to urge the tensionable fixation member into (or farther into) the tapered extension of the cleat so that the tensionable fixation member is wedged or otherwise captured by the tapered extension. This provides the primary or provisional locking of the tensionable fixation member to the base referred to above. In some embodiments, the base may include one or more notches or recesses at the intersection of one or more lateral openings and the bottom surface, the recesses being configured to improve clearance for certain types of needles during use. In some embodiments, the recesses also enable use of a longer cleat and/or fixation channel.

In some embodiments, the base further includes one or more vertical openings extending through the base from the top surface to the bottom surface. By way of example, the vertical openings may be configured to enable passage of a purchase element (not shown) configured to extend through the vertical opening and into a bone segment (or fixation plate or prosthesis) that the base is positioned against in order to secure the base to the bone prior to performing the repair procedure (or after commencement of the repair procedure if determined to be necessary). In some embodiments, the purchase element may be (by way of example only) a bone screw, pin, wire, tack, or any member capable of securing the base to a bone. In some embodiments, the base includes a plurality of vertical openings. In some embodiments, the plurality of vertical openings may be interspersed between the cleats. In some embodiments, the number of vertical openings may be less than the number of cleats. In some embodiments, the vertical openings may be used to pass additional tensionable fixation members therethrough.

In some embodiments, the bottom surface may be planar. In some embodiments, the bottom surface may be contoured to more effectively rest against a bone segment or a prosthesis. In some embodiments, the bottom surface may have a concave curvature.

In some embodiments, the lateral surface may include a plurality of concave recesses (or other contoured surface) disposed between the lateral openings. By way of example, the concave recesses may be configured to provide friction for a user during use.

In some embodiments, the locking element has an outer perimeter shape corresponding to the perimeter shape of the base. In some embodiments, the locking element may be sized to fit on top of the base such that the outer perimeter dimensions of the locking element and base are substantially similar. In some embodiments, the locking element has a proximal or top surface, a distal or bottom surface, and a central opening extending through the locking element from the top surface to the bottom surface along the central axis. In some embodiments, the central opening includes a threaded lumen configured to threadedly receive the threaded post of the lock screw to enable secure coupling of the locking element and the lock screw. In some embodiments, the locking element may have a perimeter recess configured to engage with the perimeter ridge of the base when the locking element is advanced onto the base. In some embodiments, one or more tensionable fixation members may be captured between the perimeter ridge and perimeter recess thereby providing the secondary locking feature as described above.

In some embodiments, the lock screw may include a head, a threaded post extending distally from the head, a neck recess positioned between the head and the threaded post, and a central lumen extending through the lock screw along the central axis. In some embodiments, the neck recess extends circumferentially around the perimeter of the threaded post and includes a smooth surface. In some embodiments, the locking element is coupled to the lock screw by threadedly advancing the lock screw through the central opening of the locking element. By way of example, the threaded post may advance fully through the central opening until the neck recess is positioned within the threaded opening. At this point, the locking element is captured by the neck recess, preventing dissociation of the locking element from the lock screw while allowing rotational movement of the lock screw relative to the locking element when the locking element is positioned within the neck recess. This feature ensures that the lock screw may rotate relative to the locking element, but the locking element does not rotate against the tensionable fixation members during final tightening of the locking element, which could introduce wear due to friction on the tensionable fixation members as well as potentially adversely impact the tension applied to the tensionable fixation members. In some embodiments, the central lumen is sized and shaped to facilitate engagement with a driver tool. For example, the central lumen has a hexagon-shaped perimeter shape, however shapes are possible. By way of example, the threaded post is configured to threadedly engage the threaded surface of the central recess of the base. In some embodiments, actuation of the lock screw (e.g., by a driver tool) advances the lock screw into the central opening, which causes the head of the lock screw to exert a compressive force on the locking element, which in turn exerts a compressive force on the tensionable fixation members within the locking interface described above.

In some embodiments, the system of the present disclosure may be used in soft tissue-to-bone and/or bone-to-bone repair. To use the system, a user may first place the base on a portion of a bone to which the damaged soft tissue and/or bone is to be attached (e.g., or the base may be secured to a fixation plate or prosthesis as described below). The user may then opt to secure the placement on the bone by inserting one or more purchase members (e.g., screws, wires, etc.) through one or more vertical openings to ensure the base remains in place during use. At this point, the user may begin the repair by attaching a fixation end of at least one tensionable fixation member to the damaged soft tissue or bone (e.g., by passing the fixation end through the tissue, bone, or other member). The free end of the attached tensionable fixation member may be associated with a chaperone member (e.g., needle, passing loop, etc.) and then passed through a lateral opening of the base, for example a lateral opening that is closest to and/or oriented toward the damaged tissue/bone to ensure the most efficient path to reduction. The free end is then passed through the fixation channel and cleat associated with the selected lateral opening such that the free end extends proximally from the top surface of the base. The use may then adjust the tension in the tensionable fixation member to a desired level, at which point the user may then urge the tensionable fixation member into the tapered extension of the cleat while maintaining the desired tension in the tensionable fixation member to provisionally lock the tensionable fixation member to the base by wedging or pinching the tensionable fixation member within the cleat.

This process may be repeated multiple times with multiple tensionable fixation members passing through distinct lateral opening/cleat combinations. In some embodiments, the fixation ends of the additional tensionable fixation members may be attached to the same damaged tissue and/or bone and/or prosthesis or distinct damaged tissue and/or bone and/or prosthesis. Once all the tensionable fixation members have been provisionally locked within their respective cleats, the user may then apply the secondary lock by coupling the locking element to the base. Prior to coupling the locking element to the base, the locking element may by coupled to the lock screw as described above such that the locking element is positioned within the neck recess. In some embodiments, the locking element and lock screw may be provided in an already coupled state so the user does not have to perform this step during the procedure. The secondary locking step may be accomplished by aligning the threaded post of the lock screw with the central opening of the base, and then rotationally advancing the lock screw into the central opening until the locking element contacts the tensionable fixation members. Final tightening (e.g., the secondary lock) is then accomplished by further advancement of the lock screw. Once the locking element is in contact with the tensionable fixation members, further rotation of the lock screw will cause the locking element to compress the tensionable fixation members without rotating. This will effectively pinch the tensionable fixation members within the locking interface between the perimeter ridge of the base and the perimeter recess of the locking element. At this point the construct is secured and excess free ends of the tensionable fixation members may be removed or optionally tied in one or more knots.

In some embodiments, if the user determines at any point that any of the tensionable fixation members need to be re-tensioned, the lock screw may be rotated in the opposite direction (e.g., counterclockwise) which will release the locking element from the tensionable fixation members, enabling the locking element to be temporarily removed. Once this happens, the tensionable fixation members should still be provisionally locked under the original applied tension by virtue of being wedged within their respective cleats. To adjust tension in any one tensionable fixation member, the user merely locates the tensionable fixation member at issue, pulls the tensionable fixation member toward the central opening to decouple the tensionable fixation member from the tapered extension of the cleat, adjust the tension to a desired level, and re-wedge the tensionable fixation member within the tapered extension of the cleat. This can be repeated independently for any tensionable fixation member that needs adjusting.

In some embodiments, the locking element may be sized to fit on top of the base such that the outer perimeter dimensions of the locking element are similar but smaller than the outer perimeter dimensions of the base, and the locking element is configured to flex radially outward as a result of compressive forces imparted upon it by the lock screw during final tightening. In some embodiments, the base may have a perimeter ridge or lip having a medial surface configured to engage with the perimeter edge of the locking element when the locking element is advanced onto the base and radially flexed. In some embodiments, the medial surface of the perimeter ridge of the base and perimeter edge of the locking element cooperate to form a locking interface configured to capture and pinch the tensionable fixation members therebetween upon actuation of the lock screw, thereby providing the secondary locking feature.

In some embodiments, the base may have one or more single perimeter recesses configured to engage with a single perimeter ridge of the locking element when the locking element is advanced onto the base. In some embodiments, the base may have one or more double perimeter recesses configured to engage with a double perimeter ridge of the locking element when the locking element is advanced onto the base. In some embodiments, one or more tensionable fixation members may be captured or pinched between the single perimeter recesses and single perimeter ridges and/or the double perimeter recesses and the double perimeter ridges thereby providing a secondary locking feature. In some embodiments, the single perimeter recesses of the base and single perimeter ridges of the locking element, and/or the double perimeter recesses of the base and double perimeter ridges of the locking element cooperate to form locking interfaces configured to capture and compress the tensionable fixation members therebetween upon actuation of the lock screw, thereby providing the secondary locking feature.

In some embodiments, the tensionable fixation member passes between the locking element and the base and is knotlessly secured by via pinching between locking surfaces on the locking element and locking surfaces on the base.

In some embodiments, the multi-strand knotless tensionable fixation system may include a coupling element configured to couple the base to bone, an orthopedic fixation plate, and/or an artificial prosthesis.

In some embodiments, the base may have one or more transverse channels extending therethrough, the transverse channels configured to enable passage of tensionable fixation members from one portion of the base to another portion of the base.

In some embodiments, the base may include recesses formed on the bottom surface surrounding vertical through-holes, the recesses being configured to provide clearance for bone screw heads that may be protruding from a fixation plate to which the base is coupled.

In some embodiments, the multi-strand knotless tensionable fixation system may further include a tunnel plate coupled to the base. In some embodiments, the tunnel plate may have a plurality of transverse and/or angled channels formed therethrough to enable passage of a tensionable fixation member from one side of the system to another side of the system.

In some embodiments, the multi-strand knotless tensionable fixation system may be coupled to a fixation plate, prosthesis, or other artificial implant using a coupling post.

In some embodiments, the base includes a plurality of fixation channels positioned between each cleat and extending radially inward (toward the central opening). By way of example, each fixation channel comprises a compression surface which may be planar or curved and is configured to interact with a distal protrusion of the locking element to form a locking interface that captures the tensionable fixation member in the secondary lock feature of the system. In some embodiments, each fixation channel may be associated with a pair of cleats such that a single distal protrusion may lock one or two tensionable fixation members within a single fixation channel (e.g., a tensionable fixation member engaged with a cleat located on either side of the fixation channel). Thus, the base is configured such that, after securing or otherwise associating a fixation end (or portion) of a tensionable fixation member to a tissue or bone segment to be repaired (or prosthesis), the free end (or a first free end) of the tensionable fixation member may be passed (e.g., using a chaperone element such as a needle, passing loop, or guide wire, etc.) into the base through the ingress opening, then guided laterally through one of the associated tapered wedge recesses of the cleat, and then into the fixation channel such that the free end extends away from the fixation channel. After pulling on the free end to apply a desired amount of tension to the tensionable fixation member, the user may then pull radially on the tensionable fixation member to urge the tensionable fixation member into (or farther into) the tapered wedge recess of the cleat so that the tensionable fixation member is wedged or otherwise captured by the tapered wedge recess. This provides the primary or provisional locking of the tensionable fixation member to the base referred to above.

In some embodiments, the base may include an annular sloped surface surrounding the central opening and configured to deflect a chaperone element (such as a needle, passing loop, or guide wire used to usher the tensionable fixation member through the cleat) away from the central opening to make it easier for a user to maneuver during use.

In some embodiments, the bottom surface may include on or more utility flanges formed therein. By way of example, the utility flanges are configured to engage with a tensionable fixation member, for example a user may loop a tensionable fixation member around a utility flange to change the direction of the tensionable fixation member within the inner cavity of the base.

In some embodiments, the base includes a plurality of vertical openings. In some embodiments, the plurality of vertical openings may be interspersed between the cleats. For example, in such a case, the vertical openings positioned between the cleats (and within a fixation channel) may serve as an anchor point for a fixation portion of a tensionable fixation member, which is then threaded through or wrapped around an object (e.g., muscle, tendon, bone, prosthesis, or other member) and returned to the base such that the free ends are secured to the base using the cleats and locking element.

In some embodiments, the locking element may have a plurality of distal protrusions extending distally from the bottom surface and configured to nest within the fixation channels of the base when the locking element is advanced onto the base. By way of example, the plurality of distal protrusions may be distributed in a radial manner adjacent the outer perimeter edge of the locking element, in spatial alignment with the fixation channels of the base when the locking element is applied to the base. In some embodiments, one or more tensionable fixation members may be captured between the distal protrusions and compression surface of the fixation channel (which collectively form locking interfaces) thereby providing the secondary locking feature as described above. In some embodiments, the distal protrusions may have variable height dimensions to accommodate different types of tensionable fixation members, including but not limited to (and by way of example only) surgical sutures, tapes, wires, etc. In some embodiments, the distal protrusions have a width dimension substantially similar to (but slightly smaller than) the width dimension of the fixation channels, so that the distal protrusions may be snugly received within the fixation channels to ensure alignment during association of the locking element with the base and to act as a counter torque feature during actuation of the lock screw during final tightening, reducing or eliminating rotational friction that might otherwise cause rotational misalignment of the locking element relative to the base.

In some embodiments, the base and locking element may have an overall concave curvature. By way of example, this configuration can be advantageous to ensure a more secure interaction with a contoured anatomy, curved plate, or curved prosthesis.

In some embodiments, any of the multi-strand knotless tensionable fixation systems described herein may be used in a method of securing at least two objects under tension with at a plurality of tensionable fixation members.

In some embodiments, any of the multi-strand knotless tensionable fixation systems described herein may be used in a method of securing at least two portions of a singular object under tension with at a plurality of tensionable fixation members.

In some embodiments, any of the multi-strand knotless tensionable fixation systems described herein may be used in a method of securing at least an object under tension with at least one tensionable fixation member.

As additional description to the embodiments described below, the present disclosure describes the following embodiments.

Embodiment 1 is a multi-strand knotless tensionable fixation system, comprising: a plurality of tensionable fixation members, each tensionable fixation member of the plurality of tensionable fixation members having at least one free end and a target engagement portion; a base member having a proximal surface, a distal surface, a laterally facing perimeter surface, a central opening extending between the proximal and distal surface, a first locking interface feature positioned on the proximal surface, and a plurality of provisional locking cleats formed within the proximal surface; a locking element having a proximal surface, a distal surface, a central opening extending between the proximal and distal surfaces, and a second locking interface feature positioned on the distal surface; and a compression member configured to extend through the central opening of the locking element and into the central opening of the base member, the compression member having a radial flange configured to engage the proximal surface of the locking element; wherein each provisional locking cleat of said plurality of provisional locking cleats is configured to hold fast an associated one of the plurality of tensionable fixation members extending between the provisional locking cleat and a target object under tension; wherein the compression member is configured such that actuation of the compression member causes the compression member to translate distally within the central opening of the base member such that the radial flange exerts a compressive force on the locking element; and wherein the first locking interface feature and second locking interface feature are configured to cooperate with one another to pinch said plurality of tensionable fixation members associated with said plurality of provisional locking cleats upon actuation of the compression member.

Embodiment 2 is the system of embodiment 1, wherein the compression member is a lock screw having a head and a threaded shank, and the head comprises the radial flange.

Embodiment 3 is the system of embodiments 1 or 2, wherein each provisional locking cleat of the plurality of provisional locking cleats comprises a tapered wedge member configured to engage one of said plurality of tensionable fixation members.

Embodiment 4 is the system of any of embodiments 1 through 3, wherein the base member further comprises a plurality of lateral openings formed in said laterally facing surface, each one of said plurality of lateral openings providing access into a corresponding one of said plurality of provisional locking cleats.

Embodiment 5 is the system of any of embodiments 1 through 4, wherein each one of the plurality of lateral openings is configured to enable passage of one of said plurality of tensionable fixation members therethrough.

Embodiment 6 is the system of any of embodiments 1 through 5, wherein the base includes one or more transverse channels extending therethrough.

Embodiment 7 is the system of any of embodiments 1 through 6, wherein the first locking interface feature is a raised portion of the proximal surface extending around the perimeter of the base member.

Embodiment 8 is the system of any of embodiments 1 through 7, wherein the second locking interface features is a recess of the distal surface extending around the perimeter of the locking element.

Embodiment 9 is the system of any of embodiments 1 through 8, further comprising a coupling member configured to couple the base member to at least one of a fixation plate, artificial prosthesis, and bone.

Embodiment 10 is the system of any of embodiments 1 through 9, wherein the second locking interface comprises a distal facing protrusion extending from the distal surface of the locking element.

Embodiment 11 is a method of securing one or more objects with a multi-strand tensionable fixation system, comprising the steps of: positioning a multi-strand tensionable fixation system near one or more target objects to be secured under tension, the multi-strand tensionable fixation system comprising: a plurality of tensionable fixation members, each tensionable fixation member of the plurality of tensionable fixation members having at least one free end and a target engagement portion; a base member having a proximal surface, a distal surface, a laterally facing perimeter surface, a central opening extending between the proximal and distal surface, a first locking interface feature positioned on the proximal surface, and a plurality of provisional locking cleats formed within the proximal surface, each provisional locking cleat of said plurality of provisional locking cleats configured to hold fast an associated one of said plurality of tensionable fixation members extending between the provisional locking cleat and a target object under tension; a locking element having a proximal surface, a distal surface, a central opening extending between the proximal and distal surfaces, and a second locking interface feature positioned on the distal surface; a compression member a compression member configured to extend through the central opening of the locking element and into the central opening of the base member, the compression member having a radial flange configured to engage the proximal surface of the locking element; engaging a first target object of said one or more target objects with a target engagement portion of a first tensionable fixation member of said plurality of tensionable fixation members; provisionally securing a first free end of said first tensionable fixation member within a first provisional locking cleat of said plurality of provisional locking cleats; provisionally securing a second free end of said first tensionable fixation member within a second provisional locking cleat of said plurality of provisional locking cleats; engaging a second target object of said one or more target objects with a target engagement portion of a second tensionable fixation member of said plurality of tensionable fixation members; provisionally securing a first free end of said second tensionable fixation member within a third provisional locking cleat of said plurality of provisional locking cleats; provisionally securing a second free end of said second tensionable fixation member within a fourth provisional locking cleat of said plurality of provisional locking cleats; positioning the locking element on the base member such that the provisionally secured first and second free ends of said first tensionable fixation member and first and second free ends of said second tensionable fixation member are positioned between the first locking interface feature of the base member and the second locking interface feature of the locking element; and advancing the compression member through the central opening of the locking element and into the central opening of the base member such that the radial flange contacts the proximal surface of the locking element; and actuating the compression member such that the compression member translates distally within the central opening of the base member and the radial flange exerts a compressive force on the engaged proximal surface of the locking element, thereby causing the provisionally secured first and second free ends of said first tensionable fixation member and first and second free ends of said second tensionable fixation member to be pinched between the first locking interface feature of the base member and the second locking interface feature of the locking element to achieve a final locked state.

Embodiment 12 is the method of embodiment 11, wherein the step of provisionally securing a first free end of said first tensionable fixation member within a first provisional locking cleat of said plurality of provisional locking cleats further comprises the sub-steps of: passing the first free end of said first tensionable fixation member into the first provisional locking cleat of said plurality of provisional locking cleats; applying a desired amount of tension to the first free end of said first tensionable fixation member; and pulling on said free end to wedge said first free end of said first tensionable fixation member into a tapered wedge portion of said first provisional locking cleat under said desired amount of tension.

Embodiment 13 is the method of embodiments 11 or 12, wherein the step of provisionally securing a second free end of said first tensionable fixation member within a second provisional locking cleat of said plurality of provisional locking cleats further comprises the sub-steps of: passing the second free end of said first tensionable fixation member into the second provisional locking cleat of said plurality of provisional locking cleats; applying a desired amount of tension to the second free end of said first tensionable fixation member; and pulling on said second free end to wedge said second free end of said first tensionable fixation member into a tapered wedge portion of said second provisional locking cleat under said desired amount of tension.

Embodiment 14 is the method of any of embodiments 11 through 13, wherein the step of provisionally securing a first free end of said second tensionable fixation member within a third provisional locking cleat of said plurality of provisional locking cleats further comprises the sub-steps of: passing the second free end of said first tensionable fixation member into the third provisional locking cleat of said plurality of provisional locking cleats; applying a desired amount of tension to the first free end of said second tensionable fixation member; and pulling on said first free end of said second tensionable fixation member to wedge said first free end of said second tensionable fixation member into a tapered wedge portion of said third provisional locking cleat under said desired amount of tension.

Embodiment 15 is the method of any of embodiments 11 through 14, wherein the step of provisionally securing a second free end of said second tensionable fixation member within a fourth provisional locking cleat of said plurality of provisional locking cleats further comprises the sub-steps of: passing the second free end of said second tensionable fixation member into the fourth provisional locking cleat of said plurality of provisional locking cleats; applying a desired amount of tension to the second free end of said second tensionable fixation member; and pulling on said second free end of said second tensionable fixation member to wedge said second free end of said second tensionable fixation member into a tapered wedge portion of said fourth provisional locking cleat under said desired amount of tension.

Embodiment 16 is the method of any of embodiments 11 through 15, wherein said first and second provisional locking cleats are positioned adjacent to one another.

Embodiment 17 is the method of any of embodiments 11 through 16, wherein the third and fourth provisional locking cleats are positioned adjacent to one another.

Embodiment 18 is the method of any of embodiments 11 through 17, further comprising the step of coupling the base member to an orthopedic fixation plate, artificial prosthesis, or bone segment.

Embodiment 19 is the method of any of embodiments 11 through 18, wherein the compression member is a lock screw having a head and a threaded shank, and the head comprises the radial flange.

Embodiment 20 is the method of any of embodiments 11 through 19, wherein each provisional locking cleat of the plurality of provisional locking cleats comprises a tapered wedge member configured to engage one of said plurality of tensionable fixation members.

Embodiment 21 is the method of any of embodiments 11 through 20, wherein the base member further comprises a plurality of lateral openings formed in said laterally facing surface, each one of said plurality of lateral openings providing access into a corresponding one of said plurality of provisional locking cleats.

Embodiment 22 is the method of any of embodiments 11 through 21, wherein each one of the plurality of lateral openings is configured to enable passage of one of said plurality of tensionable fixation members therethrough.

Embodiment 23 is the method of any of embodiments 11 through 22, wherein the base includes one or more transverse channels extending therethrough.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a partially exploded top perspective view of an example of a multi-strand knotless tensionable fixation system, according to some embodiments;

FIG. 2 is a partially exploded bottom perspective view of the multi-strand knotless tensionable fixation system of FIG. 1, according to some embodiments;

FIG. 3 is a perspective view of the multi-strand knotless tensionable fixation system of FIG. 1, according to some embodiments;

FIG. 4 is a top perspective view of an example of a base member forming part of the multi-strand knotless tensionable fixation system of FIG. 1, according to some embodiments;

FIG. 5 is a bottom perspective view of the base member of FIG. 4, according to some embodiments;

FIG. 6 is a top plan view of the base member of FIG. 4, according to some embodiments;

FIG. 7 is a side plan view of the base member of FIG. 4, according to some embodiments;

FIG. 8 is a top perspective view of an example of a locking element forming part of the multi-strand knotless tensionable fixation system of FIG. 1, according to some embodiments;

FIG. 9 is a bottom perspective view of the locking element of FIG. 9, according to some embodiments;

FIG. 10 is a bottom plan view of the locking element of FIG. 9, according to some embodiments;

FIG. 11 is a bottom perspective view of an example of a lock screw forming part of the multi-strand knotless tensionable fixation system of FIG. 1, according to some embodiments;

FIG. 12 is a top perspective view of the lock screw of FIG. 11, according to some embodiments;

FIG. 13 is a side plan view of the lock screw of FIG. 11, according to some embodiments;

FIG. 14 is a top plan view of the lock screw of FIG. 11, according to some embodiments;

FIG. 15 is a top perspective view of another example of a multi-strand knotless tensionable fixation system, according to some embodiments;

FIG. 16 is a bottom perspective view of the multi-strand knotless tensionable fixation system of FIG. 15, according to some embodiments;

FIG. 17 is a partially exploded perspective view of the multi-strand knotless tensionable fixation system of FIG. 15, according to some embodiments;

FIG. 18 is a top plan view of the multi-strand knotless tensionable fixation system of FIG. 15, according to some embodiments;

FIG. 19 is a partially exploded side plan view of the multi-strand knotless tensionable fixation system of FIG. 15, according to some embodiments;

FIG. 20 is a perspective view of the multi-strand knotless tensionable fixation system of FIG. 15, according to some embodiments;

FIG. 21 is a top perspective view of an example of a base member forming part of the multi-strand knotless tensionable fixation system of FIG. 15, according to some embodiments;

FIG. 22 is a bottom perspective view of the base member of FIG. 21, according to some embodiments;

FIG. 23 is a top plan view of the base member of FIG. 21, according to some embodiments;

FIG. 24 is a side plan view of the base member of FIG. 21, according to some embodiments;

FIG. 25 is a top perspective view of an example of a locking element forming part of the multi-strand knotless tensionable fixation system of FIG. 15, according to some embodiments;

FIG. 26 is a bottom perspective view of the locking element of FIG. 25, according to some embodiments;

FIG. 27 is a top perspective view of another example of a multi-strand knotless tensionable fixation system, according to some embodiments;

FIG. 28 is a bottom perspective view of the multi-strand knotless tensionable fixation system of FIG. 27, according to some embodiments;

FIG. 29 is a partially exploded perspective view of the multi-strand knotless tensionable fixation system of FIG. 27, according to some embodiments;

FIG. 30 is a partially exploded bottom perspective view of the multi-strand knotless tensionable fixation system of FIG. 27, according to some embodiments;

FIG. 31 is a perspective view of the multi-strand knotless tensionable fixation system of FIG. 27, according to some embodiments;

FIG. 32 is a top perspective view of an example of a base member forming part of the multi-strand knotless tensionable fixation system of FIG. 27, according to some embodiments;

FIG. 33 is a bottom perspective view of the base member of FIG. 32, according to some embodiments;

FIG. 34 is a side plan view of the base member of FIG. 32, according to some embodiments;

FIG. 35 is a top plan view of the base member of FIG. 32, according to some embodiments;

FIG. 36 is a top perspective view of an example of a locking element forming part of the multi-strand knotless tensionable fixation system of FIG. 27, according to some embodiments;

FIG. 37 is a bottom perspective view of the locking element of FIG. 36, according to some embodiments;

FIG. 38 is a bottom plan view of the locking element of FIG. 36, according to some embodiments;

FIG. 39 is a perspective view of another example of a multi-strand knotless tensionable fixation system, according to some embodiments;

FIG. 40 is a bottom perspective view of the multi-strand knotless tensionable fixation system of FIG. 39, according to some embodiments;

FIG. 41 is a bottom plan view of the multi-strand knotless tensionable fixation system of FIG. 39, according to some embodiments;

FIG. 42 is a side plan view of the multi-strand knotless tensionable fixation system of FIG. 39, according to some embodiments;

FIG. 43 is a top perspective view of an example of a base member forming part of the multi-strand knotless tensionable fixation system of FIG. 39, according to some embodiments;

FIG. 44 is a bottom perspective view of the base member of FIG. 43, according to some embodiments;

FIG. 45 is a perspective view of an example of a locking element forming part of the multi-strand knotless tensionable fixation system of FIG. 39, according to some embodiments;

FIG. 46 is another perspective view of the locking element of FIG. 45, according to some embodiments;

FIG. 47 is a top perspective view of another example of a multi-strand knotless tensionable fixation system, according to some embodiments;

FIG. 48 is a bottom perspective view of the multi-strand knotless tensionable fixation system of FIG. 47, according to some embodiments;

FIG. 49 is a partially exploded perspective view of the multi-strand knotless tensionable fixation system of FIG. 47, according to some embodiments;

FIG. 50 is a perspective view of the multi-strand knotless tensionable fixation system of FIG. 47, according to some embodiments;

FIG. 51 is a bottom perspective view of the multi-strand knotless tensionable fixation system of FIG. 47, according to some embodiments;

FIG. 52 is a side plan view of the multi-strand knotless tensionable fixation system of FIG. 47, according to some embodiments;

FIG. 53 is a top perspective view of an example of a base member forming part of the multi-strand knotless tensionable fixation system of FIG. 47, according to some embodiments;

FIG. 54 is a bottom perspective view of the base member of FIG. 53, according to some embodiments;

FIG. 55 is a side perspective view of the base member of FIG. 53, according to some embodiments;

FIG. 56 is a top plan view of the base member of FIG. 53, according to some embodiments;

FIG. 57 is a cross-sectional view of the base member of FIG. 53 taken along line A—A in FIG. 55;

FIG. 58 is a top perspective view of an example of a locking element forming part of the multi-strand knotless tensionable fixation system of FIG. 47, according to some embodiments;

FIG. 59 is a bottom perspective view of the locking element of FIG. 58, according to some embodiments;

FIG. 60 is a top perspective view of an example of a coupling element forming part of the multi-strand knotless tensionable fixation system of FIG. 47, according to some embodiments;

FIG. 61 is a bottom perspective view of the coupling element of FIG. 60, according to some embodiments;

FIG. 62 is a side plan view of the coupling element of FIG. 60, according to some embodiments;

FIG. 63 is a top plan view of the coupling element of FIG. 60, according to some embodiments;

FIG. 64 is a top perspective view of an example of a lock screw forming part of the multi-strand knotless tensionable fixation system of FIG. 47, according to some embodiments;

FIG. 65 is a bottom perspective view of the lock screw of FIG. 64, according to some embodiments;

FIG. 66 is a side plan view of the lock screw of FIG. 64, according to some embodiments;

FIG. 67 is a top plan view of the lock screw of FIG. 64, according to some embodiments;

FIGS. 68-69 are perspective and exploded perspective views, respectively, of the multi-strand knotless tensionable fixation system of FIG. 47 coupled with a bone plate, according to some embodiments;

FIG. 70 is another exploded perspective view of the multi-strand knotless tensionable fixation system of FIG. 47 coupled with a bone plate, according to some embodiments;

FIG. 71 is a side sectional view of the multi-strand knotless tensionable fixation system of FIG. 47 coupled with a bone plate, according to some embodiments;

FIG. 72 is a top perspective view of another example of a base member forming part of the multi-strand knotless tensionable fixation system of FIG. 47, according to some embodiments;

FIG. 73 is a top plan view of the base member of FIG. 72, according to some embodiments;

FIG. 74 is a bottom perspective view of the base member of FIG. 72, according to some embodiments;

FIG. 75 is a bottom plan view of the base member of FIG. 72, according to some embodiments;

FIG. 76 is a side plan view of a multi-strand knotless tensionable fixation system of FIG. 47 coupled with an example of a secondary tunnel plate, according to some embodiments;

FIG. 77 is an exploded perspective view of the multi-strand knotless tensionable fixation system coupled with an example of a secondary tunnel plate of FIG. 76, according to some embodiments;

FIG. 78 is a perspective view of the secondary tunnel plate of FIG. 76, according to some embodiments;

FIG. 79 is a side plan view of the secondary tunnel plate of FIG. 76, according to some embodiments;

FIG. 80 is a sectional view of the secondary tunnel plate of FIG. 76 taken along line B-B of FIG. 79, according to some embodiments;

FIG. 81 is a plan view of a multi-strand knotless tensionable fixation system of FIG. 47 coupled with an example of a surgical prothesis, according to some embodiments;

FIG. 82 is a perspective view of another example of a multi-strand knotless tensionable fixation system coupled with an example of a bone plate, according to some embodiments;

FIG. 83 is an exploded view of the multi-strand knotless tensionable fixation system coupled with a bone plate of FIG. 82, according to some embodiments;

FIG. 84 is an exploded view of the multi-strand knotless tensionable fixation system FIG. 82, shown without the bone plate, according to some embodiments;

FIG. 85 is a perspective view of another example of a multi-strand knotless tensionable fixation system, according to some embodiments;

FIG. 86 is an exploded perspective view of the multi-strand knotless tensionable fixation system of FIG. 85, according to some embodiments;

FIG. 87 is another perspective view of the multi-strand knotless tensionable fixation system of FIG. 85, according to some embodiments;

FIG. 88 is another exploded perspective view of the multi-strand knotless tensionable fixation system of FIG. 85, according to some embodiments;

FIGS. 89-90 are perspective views of an example of a base member forming part of the multi-strand knotless tensionable fixation system of FIG. 85, according to some embodiments;

FIGS. 91-92 are side plan views of the base member of FIG. 89, according to some embodiments;

FIG. 93 is a bottom perspective view of the base member of FIG. 89, according to some embodiments;

FIG. 94 is a sectional view of the base member of FIG. 89 taken along line C—C of FIG. 91, according to some embodiments;

FIGS. 95-96 are top perspective and bottom perspective views, respectively, of another an example of a base member forming part of the multi-strand knotless tensionable fixation system of FIG. 85, according to some embodiments;

FIGS. 97-98 are top perspective and bottom perspective views, respectively, of another an example of a base member forming part of the multi-strand knotless tensionable fixation system of FIG. 85, according to some embodiments;

FIGS. 99-102 are bottom perspective, top perspective, top plan, and bottom plan views, respectively, of an example of a locking element forming part of the multi-strand knotless tensionable fixation system of FIG. 85, according to some embodiments;

FIGS. 103-104 are perspective views of another example of a base member forming part of the multi-strand knotless tensionable fixation system of FIG. 85, according to some embodiments;

FIG. 105 is a top plan view of the base member of FIG. 103, according to some embodiments;

FIG. 106 is a side perspective view of the base member of FIG. 103, according to some embodiments;

FIG. 107 is a side plan view of the base member of FIG. 103, according to some embodiments;

FIG. 108 is a sectional view of the base member of FIG. 103 taken along line D-D of FIG. 107, according to some embodiments;

FIGS. 109-110 are side plan and bottom plan views, respectively, of another example of a locking element forming part of the multi-strand knotless tensionable fixation system of FIG. 85, according to some embodiments;

FIG. 111 is a partially exploded perspective view of another example of a multi-strand knotless tensionable fixation system, according to some embodiments;

FIGS. 112-113 are exploded top perspective and exploded bottom perspective views, respectively, of the multi-strand knotless tensionable fixation system of FIG. 111, according to some embodiments;

FIGS. 114-115 are top perspective views of an example of a base member forming part of the multi-strand knotless tensionable fixation system of FIG. 111, according to some embodiments;

FIGS. 116-117 are bottom perspective and top plan views, respectively, of the base member of FIG. 114, according to some embodiments;

FIGS. 118-119 are perspective and bottom plan views, respectively, of a locking element forming part of the multi-strand knotless tensionable fixation system of FIG. 111, according to some embodiments;

FIGS. 120-121 are exploded perspective views of an example of a curved variation of the multi-strand knotless tensionable fixation system of FIG. 111, according to some embodiments;

FIGS. 122-123 are perspective and side plan views, respectively, of a base member forming part of the curved variation of FIG. 120, according to some embodiments;

FIGS. 124-125 are bottom plan and side plan views, respectively, of a locking element forming part of the curved variation of FIG. 120, according to some embodiments;

FIGS. 126-127 are exploded top perspective and exploded bottom perspective views, respectively of another variation of the multi-strand knotless tensionable fixation system of FIG. 111, according to some embodiments;

FIGS. 128-130 are perspective, side plan, and exploded perspective views, respectively, of another variation of the multi-strand knotless tensionable fixation system of FIG. 111 coupled to a bone plate, according to some embodiments;

FIG. 131 is a flowchart depicting several steps in a method of securing at least two objects under tension using the multi-strand knotless tensionable fixation system of FIG. 1, according to some embodiments;

FIGS. 132-136 are block diagrams depicting several steps of the method of FIG. 131, according to some embodiments;

FIG. 137 is a flowchart depicting several steps in a method of securing at least two portions of a singular object under tension using the multi-strand knotless tensionable fixation system of FIG. 85, according to some embodiments;

FIGS. 138-142 are block diagrams depicting several steps of the method of FIG. 131, according to some embodiments;

FIG. 143 is a flowchart depicting several steps in a method of securing at least one object under tension using the multi-strand knotless tensionable fixation system of FIG. 1, according to some embodiments; and

FIG. 144 is a block diagram depicting at least one step of the method of FIG. 143, according to some embodiments.

### DETAILED DESCRIPTION OF A PREFERRED EMBODIMENT

Illustrative embodiments of the disclosure are described below. In the interest of clarity, not all features of an actual implementation are described in this specification. It will of course be appreciated that in the development of any such actual embodiment, numerous implementation-specific decisions must be made to achieve the developers'specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. The multi-strand knotless tensionable fixation system and related methods disclosed herein boasts a variety of inventive features and components that warrant patent protection, both individually and in combination.

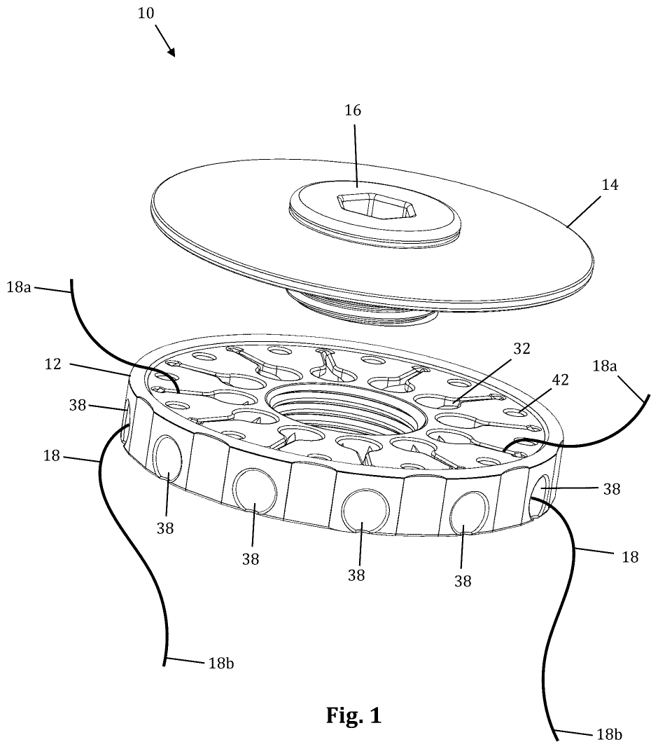

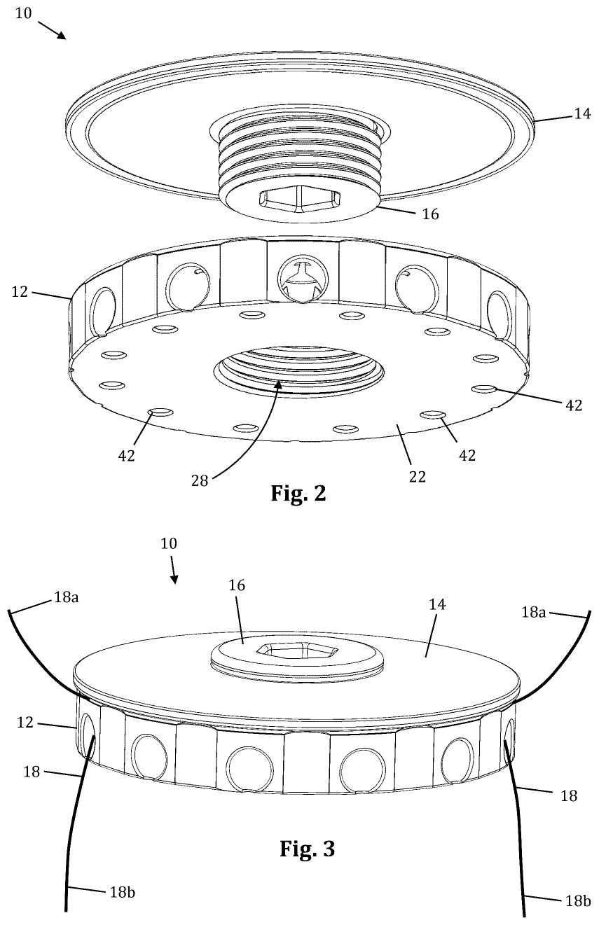

FIGS. 1-3 illustrate an example of a knotless tensionable fixation system 10 according to some embodiments of the disclosure. By way of example only, the knotless tensionable fixation system 10 (also referred to herein as “system 10”) includes a base 12, a locking element 14, a lock screw 16 and at least one tensionable fixation member 18. The system 10 of the present disclosure enables a user to achieve complex soft tissue-to-bone or bone-to-bone repair by first placing the base 12 against a first bone segment (or securing to a fixation plate or prosthesis) and then securing multiple tensionable fixation members 18 (e.g., surgical sutures, tape, wires, etc.) that may be attached to the same or different tissue or bone to be repaired (e.g., several unique tensionable fixation member strands may be attached the same tissue, or unique tensionable fixation member strands may be attached to different bone fragments or tissue segments), or to another artificial fixation member such as a prosthesis. In some embodiments, each tensionable fixation member 18 has one or more free ends 18a and a target engagement portion 18b, the target engagement portion 18b may be attached to a tissue or bone segment to be repaired or a surgical implant or prosthesis, and the tensionable fixation member 18 is passed through the base 12 such that the one or more free ends 18a extend proximally from the base 12 to enable manipulation by a user. In some embodiments, the target engagement portion 18b comprises one end of the tensionable fixation member, which then has one free end 18a. In some embodiments, the target engagement portion 18b may be a loop or middle portion of the tensionable fixation member 18, which then has two free ends 18a that may be secured by the base 12. In some embodiments, the system 10 enables each tensionable fixation member 18 to be provisionally secured to the base 12 under adequate tension while other tensionable fixation members 18 (or another strand of the same tensionable fixation member 18) are being utilized. In some embodiments, once all tensionable fixation members 18 have been provisionally secured to the base 12 under the desired tension to achieve reduction of the repair, the locking element 14 may be coupled to the base 12 and secured by the lock screw 16 under compression to provide a secondary or final lock (e.g., the provisional lock being a first lock) simultaneously to all tensionable fixation members 18 to securely lock all tensionable fixation members 18 under tension at the same time and with one single locking element 14.

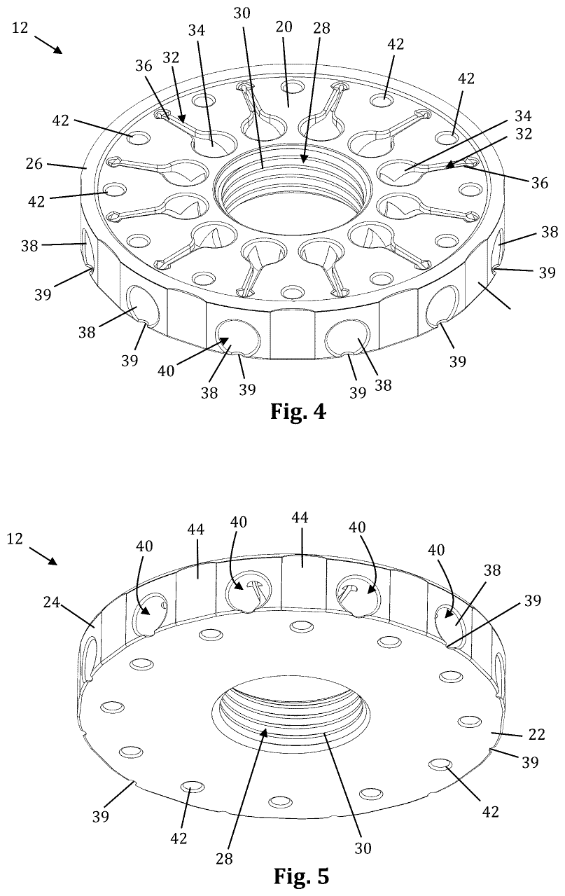

FIGS. 4-7 illustrate an example of a base 12 forming part of the system 10, according to some embodiments. By way of example, the base 12 may have any perimeter shape suitable to allow fixation of multiple tensionable fixation members 18 in multiple directions, including but not limited to circular (shown by way of example in FIGS. 1-7), semi-circular, curved, triangular, square, oval, hexagonal, octagonal, pentagonal, and the like. In some embodiments, the base 12 may have a top or proximal surface 20, a bottom or distal surface 22, and a lateral surface 24, which may be a planar surface or a curved circumferential surface as shown, depending on the perimeter shape of the base. In some embodiments, the base 12 may have a perimeter ridge or lip 26 configured to engage with the perimeter recess 58 of the locking element 14 when the locking element 14 is advanced onto the base 12. In some embodiments, the perimeter ridge 26 of the base 12 and perimeter recess 58 of the locking element 14 cooperate to form a “locking interface” configured to capture and compress the tensionable fixation members 18 therebetween upon actuation of the lock screw 16, thereby providing the secondary locking feature.

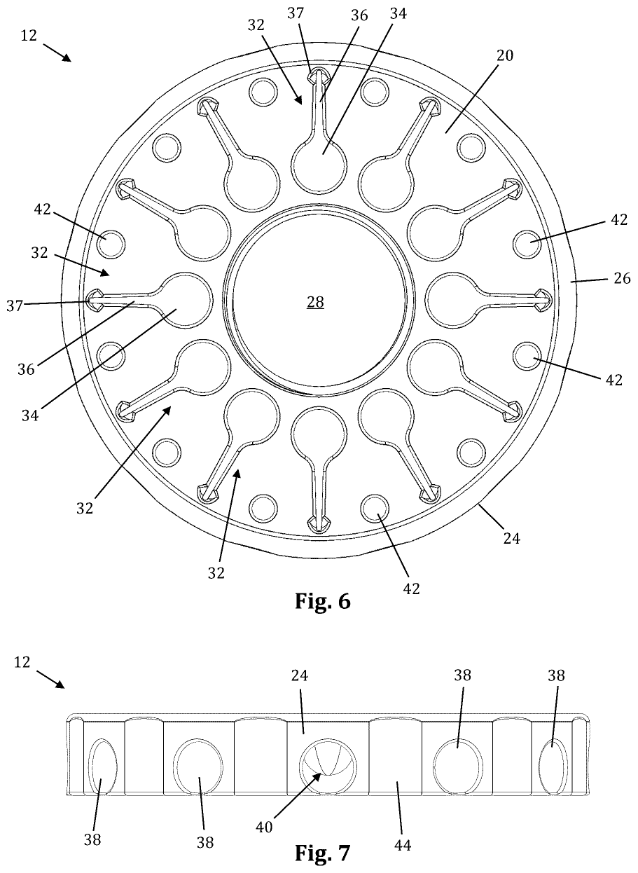

In some embodiments, the base 12 includes central opening 28 extending through the base 12 from the top surface 20 to the bottom surface 22 along a central axis. In some embodiments, the central opening 28 includes a threaded lumen 30 configured to threadedly receive the threaded post 62 of the lock screw 16 to secure the lock screw 16 to the base 12. In some embodiments, the base 12 further comprises a plurality of provisional locking cleats 32 (hereinafter “cleats 32”) distributed around the central opening 28. In some embodiments, the plurality of cleats 32 may be positioned equidistant from one another in an annular fashion, as shown by way of example in FIG. 6, however other configurations are possible. For example, in some embodiments, the plurality of cleats 32 may be grouped or bunched to one or more sides of the base 12. In some embodiments, each cleat 32 comprises a round opening 34 formed in the top surface 20 and a tapered extension 36. In some embodiments, the cleats 32 may be oriented such that the round openings 34 are positioned near the central opening 28 and the tapered extensions 36 extend toward the perimeter ridge 26. In some embodiments, the tapered extensions 36 are wider near the round opening 34 and narrower near the perimeter ridge 26. In some embodiments, the top surface 20 may include a plurality of sloped recesses 37 positioned at the radial ends of the tapered extensions 36 and configured to enable smooth passage of the tensionable fixation member 18 from the base 12.

In some embodiments, the base 12 includes a plurality of lateral openings 38 formed in the lateral surface 24 with each lateral opening 38 providing access to a fixation channel 40 extending diagonally (toward the top surface 20) into the base 12. In some embodiments, each lateral opening 38 is aligned with a unique cleat 32 such that the associated fixation channel 40 extends diagonally between the lateral opening 38 and the round opening 34 of the cleat 32, with the tapered extension 36 being open to the fixation channel 40. Thus, the base 12 is configured such that, after securing a fixation end (or portion) 18b of a tensionable fixation member 18 to a tissue or bone segment to be repaired, the free end(s) 18a of the tensionable fixation member 18 may be passed (e.g., using a chaperone element such as a needle, passing loop, or guide wire, etc.) into the base 12 through the lateral opening 38, along the diagonal fixation channel 40, and emerging through the top surface 20 through the associated cleat 32, either by way of the round opening 34 or a wider portion of the tapered extension 36. After pulling on the free end 18a to apply a desired amount of tension to the tensionable fixation member 18, the user may then pull laterally on the tensionable fixation member 18 to urge the tensionable fixation member into (or farther into) the tapered extension 36 of the cleat 32 so that the tensionable fixation member 18 is wedged or otherwise captured by the tapered extension 36. This provides the primary or provisional locking of the tensionable fixation member 18 to the base 12 referred to above. In some embodiments, the base 12 may include one or more notches or recesses 39 at the intersection of one or more lateral openings 38 and the bottom surface 22, the recesses 39 being configured to improve clearance for certain types of needles during use. In some embodiments, the recesses 39 also enable use of a longer cleat 32 and/or fixation channel 40.

In some embodiments, the base 12 further includes one or more vertical openings 42 extending through the base 12 from the top surface 20 to the bottom surface 22. By way of example, the vertical openings 42 may be configured to enable passage of a purchase element (not shown) configured to extend through the vertical opening 42 and into a bone segment (or fixation plate or prosthesis) that the base 12 is positioned against in order to secure the base 12 to the bone prior to performing the repair procedure (or after commencement of the repair procedure if determined to be necessary). In some embodiments, the purchase element may be (by way of example only) a bone screw, pin, wire, tack, or any member capable of securing the base 12 to a bone. In some embodiments, the base 12 includes a plurality of vertical openings 42. In some embodiments, the plurality of vertical openings 42 may be interspersed between the cleats 32. In some embodiments, the number of vertical openings 42 may be less than the number of cleats 32. In some embodiments, the vertical openings 42 may be used to pass additional tensionable fixation members 18 therethrough.

In some embodiments, the bottom surface 22 may be planar. In some embodiments, the bottom surface 22 may be contoured to more effectively rest against a bone segment or a prosthesis. In some embodiments, the bottom surface 22 may have a concave curvature.

In some embodiments, the lateral surface 24 may include a plurality of concave recesses 44 (or other contoured surface) disposed between the lateral openings 38. By way of example, the concave recesses 44 may be configured to provide friction for a user during use.

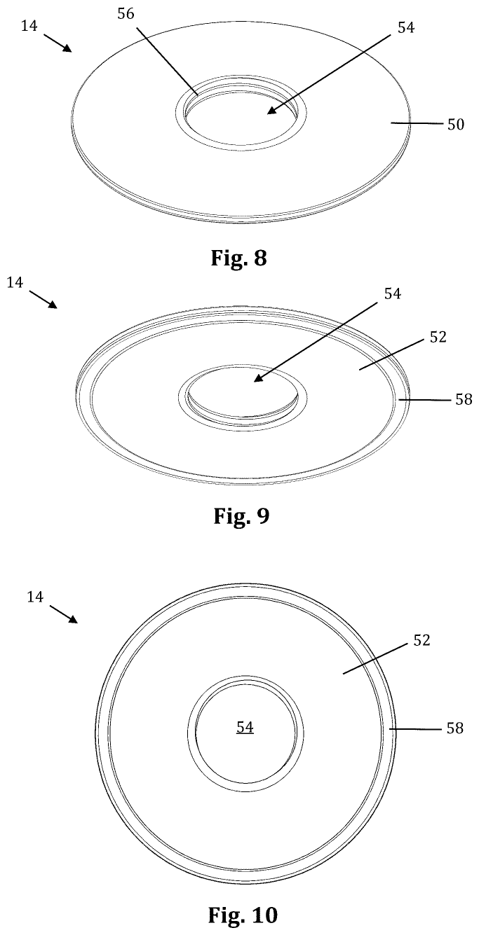

FIGS. 8-10 illustrate an example of a locking element 14, according to some embodiments. In some embodiments, the locking element 14 has an outer perimeter shape corresponding to the perimeter shape of the base 12. In some embodiments, the locking element 14 may be sized to fit on top of the base 12 such that the outer perimeter dimensions of the locking element 14 and base 12 are substantially similar. In some embodiments, the locking element 14 has a proximal or top surface 50, a distal or bottom surface 52, and a central opening 54 extending through the locking element 14 from the top surface 50 to the bottom surface 52 along the central axis. In some embodiments, the central opening 54 includes a threaded lumen 56 configured to threadedly receive the threaded post 62 of the lock screw 16 to enable secure coupling of the locking element 14 and the lock screw 16. In some embodiments, the locking element 14 may have a perimeter recess 58 configured to engage with the perimeter ridge 26 of the base 12 when the locking element 14 is advanced onto the base 12. In some embodiments, one or more tensionable fixation members 18 may be captured between the perimeter ridge 26 and perimeter recess 58 thereby providing the secondary locking feature as described above.

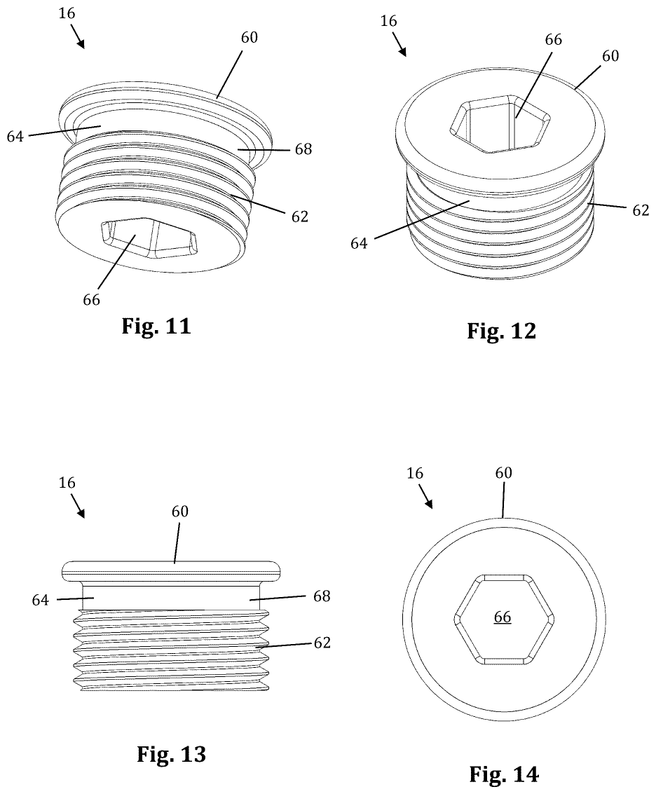

FIGS. 11-14 illustrate an example of a lock screw 16 forming part of the system 10, according to some embodiments. By way of example, the lock screw 16 may include a head 60, a threaded post 62 extending distally from the head 60, a neck recess 64 positioned between the head 60 and the threaded post 62, and a central lumen 66 extending through the lock screw 16 along the central axis. In some embodiments, the neck recess 64 extends circumferentially around the perimeter of the threaded post 62 and includes a smooth surface 68. In some embodiments, the locking element 14 is coupled to the lock screw 16 by threadedly advancing the lock screw 16 through the central opening 54 of the locking element 14. By way of example, the threaded post 62 may advance fully through the central opening 54 until the neck recess 64 is positioned within the threaded opening 54. At this point, the locking element 14 is captured by the neck recess 64, preventing dissociation of the locking element 14 from the lock screw 16 while allowing rotational movement of the lock screw 16 relative to the locking element 14 when the locking element 14 is positioned within the neck recess 64. This feature ensures that the lock screw 16 may rotate relative to the locking element 14, but the locking element 14 does not rotate against the tensionable fixation members 18 during final tightening of the locking element 14, which could introduce wear due to friction on the tensionable fixation members 18 as well as potentially adversely impact the tension applied to the tensionable fixation members 18. In some embodiments, the central lumen 66 is sized and shaped to facilitate engagement with a driver tool. For example, the central lumen 66 as shown by way of example only in FIGS. 11-14 has a hexagon-shaped perimeter shape, however shapes are possible. By way of example, the threaded post 62 is configured to threadedly engage the threaded surface 30 of the central recess 28 of the base 12. In some embodiments, actuation of the lock screw 16 (e.g., by a driver tool) advances the lock screw 16 into the central opening 28, which causes the head 60 of the lock screw 16 to exert a compressive force on the locking element 14, which in turn exerts a compressive force on the tensionable fixation members 18 within the locking interface described above.

In some embodiments, the system 10 of the present disclosure may be used in soft tissue-to-bone and/or bone-to-bone repair. To use the system 10, a user may first place the base 12 on a portion of a bone to which the damaged soft tissue and/or bone is to be attached (e.g., or the base 12 may be secured to a fixation plate or prosthesis as described below). The user may then opt to secure the placement on the bone by inserting one or more purchase members (e.g., screws, wires, etc.) through one or more vertical openings 42 to ensure the base 12 remains in place during use. At this point, the user may begin the repair by attaching a fixation end 18b of at least one tensionable fixation member 18 to the damaged soft tissue or bone (e.g., by passing the fixation end 18b through the tissue, bone, or other member). The free end 18a of the attached tensionable fixation member 18 may be associated with a chaperone member (e.g., needle, passing loop, etc.) and then passed through a lateral opening 38 of the base 12, for example a lateral opening 38 that is closest to and/or oriented toward the damaged tissue/bone to ensure the most efficient path to reduction. The free end 18a is then passed through the fixation channel 40 and cleat 32 associated with the selected lateral opening 38 such that the free end 18a extends proximally from the top surface 20 of the base 12. The use may then adjust the tension in the tensionable fixation member 18 to a desired level, at which point the user may then urge the tensionable fixation member 18 into the tapered extension 36 of the cleat 32 while maintaining the desired tension in the tensionable fixation member 18 to provisionally lock the tensionable fixation member 18 to the base 12 by wedging or pinching the tensionable fixation member 18 within the cleat 32.

This process may be repeated multiple times with multiple tensionable fixation members 18 passing through distinct lateral opening 38/cleat 32 combinations. In some embodiments, the fixation ends 18b of the additional tensionable fixation members 18 may be attached to the same damaged tissue and/or bone and/or prosthesis or distinct damaged tissue and/or bone and/or prosthesis. Once all the tensionable fixation members 18 have been provisionally locked within their respective cleats 32, the user may then apply the secondary lock by coupling the locking element 14 to the base 12. Prior to coupling the locking element 14 to the base 12, the locking element 14 may by coupled to the lock screw 16 as described above such that the locking element 14 is positioned within the neck recess 64. In some embodiments, the locking element 14 and lock screw 16 may be provided in an already coupled state so the user does not have to perform this step during the procedure. The secondary locking step may be accomplished by aligning the threaded post 62 of the lock screw 16 with the central opening 28 of the base 12, and then rotationally advancing the lock screw 16 into the central opening 28 until the locking element 14 contacts the tensionable fixation members 18. Final tightening (e.g., the secondary lock) is then accomplished by further advancement of the lock screw 16. Once the locking element 14 is in contact with the tensionable fixation members 18, further rotation of the lock screw 16 will cause the locking element 14 to compress the tensionable fixation members 18 without rotating. This will effectively pinch the tensionable fixation members 18 within the locking interface between the perimeter ridge 26 of the base 12 and the perimeter recess 58 of the locking element 14. At this point the construct is secured and excess free ends 18a of the tensionable fixation members 18 may be removed or optionally tied in one or more knots.

If the user determines at any point that any of the tensionable fixation members 18 need to be re-tensioned, the lock screw 16 may be rotated in the opposite direction (e.g., counterclockwise) which will release the locking element 14 from the tensionable fixation members 18, enabling the locking element 14 to be temporarily removed. Once this happens, the tensionable fixation members 18 should still be provisionally locked under the original applied tension by virtue of being wedged within their respective cleats 32. To adjust tension in any one tensionable fixation member 18, the user merely locates the tensionable fixation member at issue, pulls the tensionable fixation member 18 toward the central opening 28 to decouple the tensionable fixation member 18 from the tapered extension 36 of the cleat 32, adjust the tension to a desired level, and re-wedge the tensionable fixation member 18 within the tapered extension 36 of the cleat 32. This can be repeated independently for any tensionable fixation member 18 that needs adjusting.

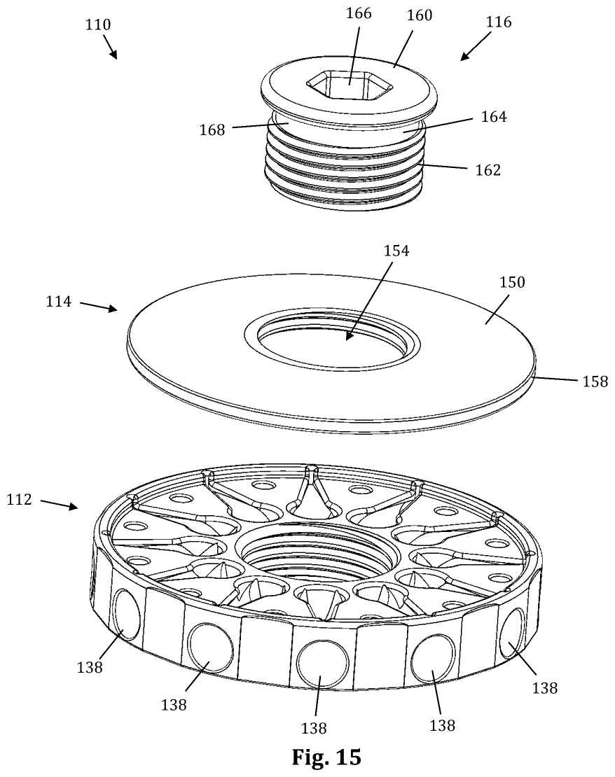

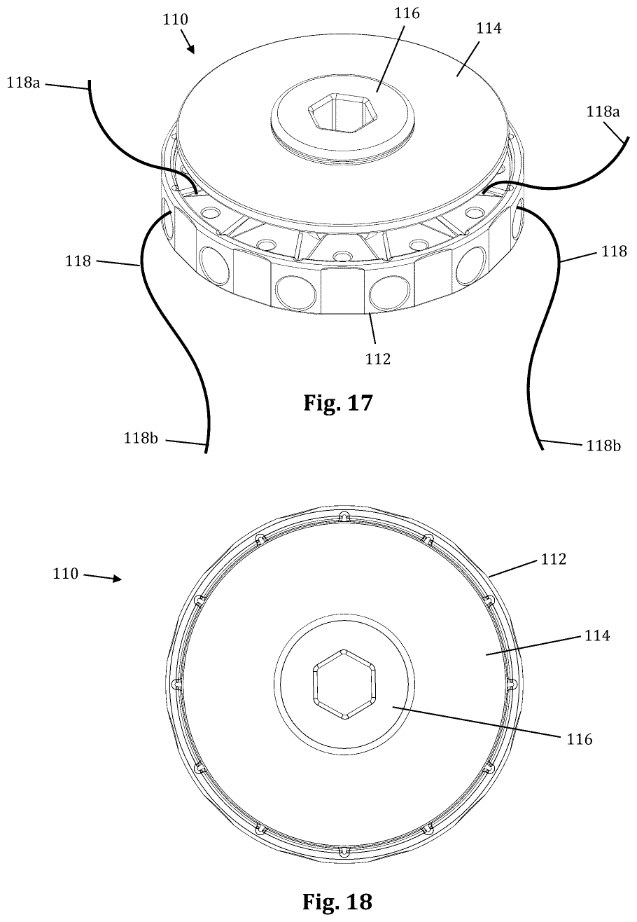

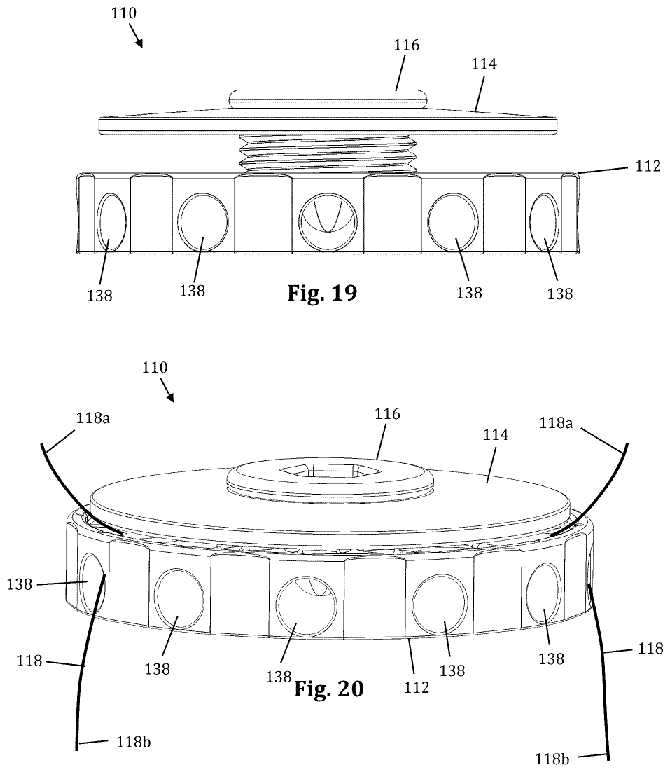

FIGS. 15-20 illustrate another example of a knotless tensionable fixation system 110 according to some embodiments of the disclosure. By way of example only, the knotless tensionable fixation system 110 (also referred to herein as “system 110”) includes a base 112, a locking element 114, a lock screw 116 and at least one tensionable fixation member 18. The system 110 of the present disclosure enables a user to achieve complex soft tissue-to-bone or bone-to-bone repair by first placing the base 112 against a first bone segment (or securing to a fixation plate or prosthesis) and then securing multiple tensionable fixation members 18 (e.g., surgical sutures, tape, wires, etc.) that may be attached to the same or different tissue or bone to be repaired (e.g., several unique tensionable fixation member strands may be attached the same tissue, or unique tensionable fixation member strands may be attached to different bone fragments or tissue segments), or to another artificial fixation member such as a prosthesis. In some embodiments, each tensionable fixation member 18 has one or more free ends 18a and a target engagement portion 18b, the target engagement portion 18b may be attached to a tissue or bone segment to be repaired or a surgical implant or prosthesis, and the tensionable fixation member 18 is passed through the base 112 such that the one or more free ends 18a extend proximally from the base 112 to enable manipulation by a user. In some embodiments, the target engagement portion 18b comprises one end of the tensionable fixation member, which then has one free end 18a. In some embodiments, the target engagement portion 18b may be a loop or middle portion of the tensionable fixation member 18, which then has two free ends 18a that may be secured by the base 112. In some embodiments, the system 110 enables each tensionable fixation member 18 to be provisionally secured to the base 112 under adequate tension while other tensionable fixation members 18 (or another strand of the same tensionable fixation member 18) are being utilized. In some embodiments, once all tensionable fixation members 18 have been provisionally secured to the base 112 under the desired tension to achieve reduction of the repair, the locking element 114 may be coupled to the base 112 and secured by the lock screw 116 under compression to provide a secondary or final lock (e.g., the provisional lock being a first lock) simultaneously to all tensionable fixation members 18 to securely lock all tensionable fixation members 18 under tension at the same time and with one single locking element 114.

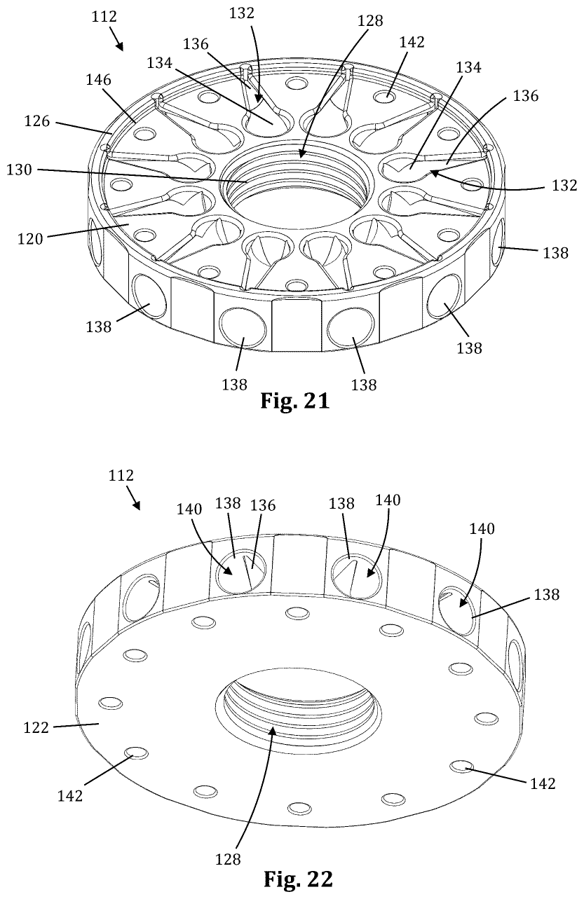

FIGS. 21-24 illustrate an example of a base 112 forming part of the system 110, according to some embodiments. By way of example, the base 112 may have any perimeter shape suitable to allow fixation of multiple tensionable fixation members 18 in multiple directions, including but not limited to circular (shown by way of example in FIGS. 15-24), semi-circular, curved, triangular, square, oval, hexagonal, octagonal, pentagonal, and the like. In some embodiments, the system 110 may have a top or proximal surface 120, a bottom or distal surface 122, and a lateral surface 124, which may be a planar surface or a circumferential surface as shown, depending on the perimeter shape of the base. In some embodiments, the base 112 may have a perimeter ridge or lip 126 having a medial surface 146 configured to engage with the perimeter edge 158 of the locking element 114 when the locking element 114 is advanced onto the base 112 and radially flexed. In some embodiments, the medial surface 146 of the perimeter ridge 126 of the base 112 and perimeter edge 158 of the locking element 114 cooperate to form a “locking interface” configured to capture and pinch the tensionable fixation members 18 therebetween upon actuation of the lock screw 116, thereby providing the secondary locking feature.

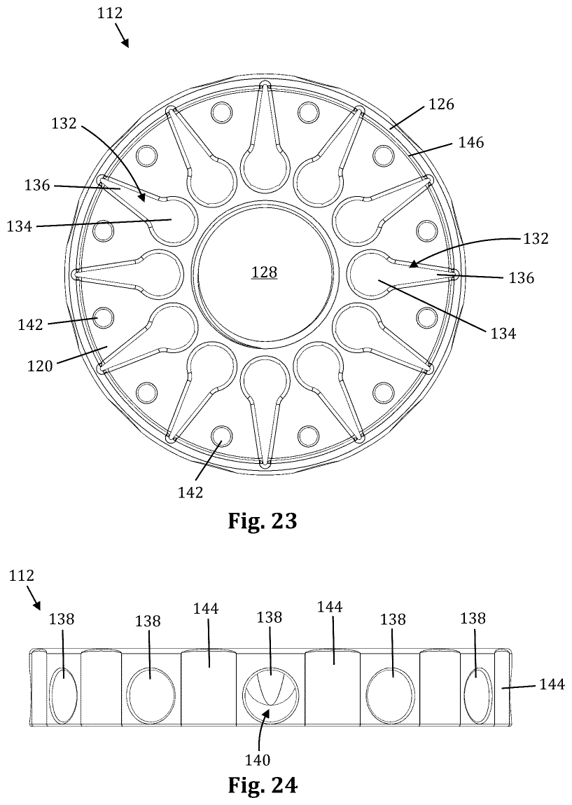

In some embodiments, the base 112 includes a central opening 128 extending through the base 112 from the top surface 120 to the bottom surface 122 along a central axis. In some embodiments, the central opening 128 includes a threaded lumen 130 configured to threadedly receive the threaded post 162 of the lock screw 116 to secure the lock screw 116 to the base 112. In some embodiments, the base 112 further comprises a plurality of cleats 132 distributed around the central opening 128. In some embodiments, the plurality of cleats 132 may be positioned equidistant from one another in an annular fashion, as shown by way of example in FIG. 23, however other configurations are possible. For example, in some embodiments, the plurality of cleats 132 may be grouped or bunched to one or more sides of the base 112. In some embodiments, each cleat 132 comprises a round opening 134 formed in the top surface 120 and a tapered extension 136. In some embodiments, the cleats 132 may be oriented such that the round openings 134 are positioned near the central opening 128 and the tapered extensions 136 extend toward the perimeter ridge 126. In some embodiments, the tapered extensions 136 are wider near the round opening 134 and narrower near the perimeter ridge 126.

In some embodiments, the base 112 includes a plurality of lateral openings 138 formed in the lateral surface 124 with each lateral opening 138 providing access to a fixation channel 140 extending diagonally (toward the top surface 120) into the base 112. In some embodiments, each lateral opening 138 is aligned with a unique cleat 132 such that the associated fixation channel 140 extends diagonally between the lateral opening 138 and the round opening 134 of the cleat 132, with the tapered extension 136 being open to the fixation channel 140. Thus, the base 112 is configured such that, after associating a fixation end 18b of a tensionable fixation member 18 with a tissue or bone segment to be repaired (or a fixation plate or a prosthesis), the free end 18a of the tensionable fixation member 18 may be passed (e.g., using a chaperone element such as a passing loop, needle, or guide wire) into the base 112 through the lateral opening 138, along the diagonal fixation channel 140, and emerging through the top surface 120 through the associated cleat 132, either by way of the round opening 134 or a wider portion of the tapered extension 136. After pulling on the free end 18a to apply a desired amount of tension to the tensionable fixation member 18, the user may then pull laterally on the tensionable fixation member 18 to urge the tensionable fixation member into (or farther into) the tapered extension 136 of the cleat 132 so that the tensionable fixation member 18 is wedged or otherwise captured by the tapered extension 136. This provides the primary or provisional locking of the tensionable fixation member 18 to the base 112 described herein.

In some embodiments, the base 112 further includes one or more vertical openings 142 extending through the base 112 from the top surface 120 to the bottom surface 122. By way of example, the vertical openings 142 may be configured to enable passage of a purchase element (not shown) configured to extend through the vertical opening 142 and into the bone segment that the base 112 is positioned against in order to secure the base 112 to the bone prior to performing the repair procedure (or after commencement of the repair procedure if determined to be necessary). In some embodiments, the purchase element may be (by way of example only) a bone screw, pin, wire, tack, or any member capable of securing the base 112 to a bone. In some embodiments, the base 112 includes a plurality of vertical openings 142. In some embodiments, the plurality of vertical openings 142 may be interspersed between the cleats 132. In some embodiments, the number of vertical openings 142 may be less than the number of cleats 132. In some embodiments, the vertical openings 142 may be used to pass additional tensionable fixation members 18 therethrough.

In some embodiments, the bottom surface 122 may be planar. In some embodiments, the bottom surface 122 may be contoured to more effectively rest against bone, a fixation plate, or a prosthesis. In some embodiments, the bottom surface 122 may be concave.

In some embodiments, the lateral surface 124 may include a plurality of concave recesses 144 (or other contoured surface) disposed between the lateral openings 138. By way of example, the concave recesses 144 may be configured to provide friction for a user during use.

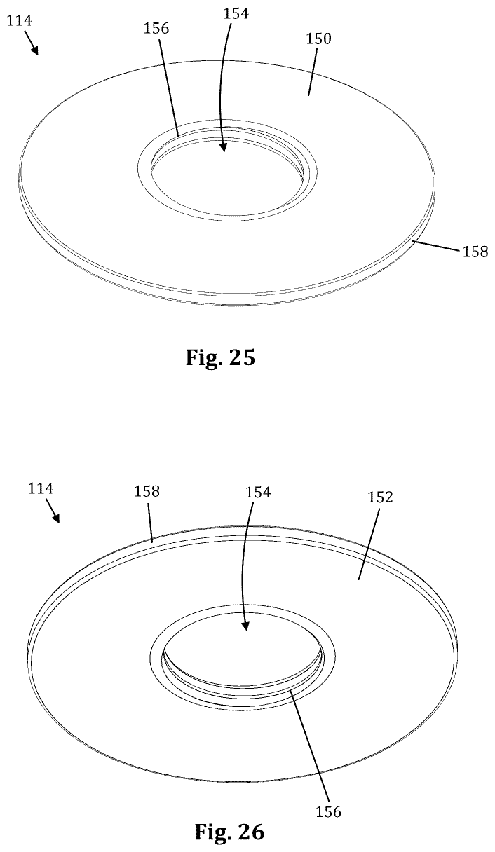

FIGS. 25-26 illustrate an example of a locking element 114, according to some embodiments. In some embodiments, the locking element 114 has an outer perimeter shape corresponding to the perimeter shape of the base 112. In some embodiments, the locking element 114 may be sized to fit on top of the base 112 such that the outer perimeter dimensions of the locking element 114 are similar but smaller than the outer perimeter dimensions of the base 112. In some embodiments, the locking element 114 has a proximal or top surface 150, a distal or bottom surface 152, and a central opening 154 extending through the locking element 114 from the top surface 150 to the bottom surface 152 along the central axis. In some embodiments, the bottom surface 152 may be concave. In some embodiments, the central opening 154 includes a threaded lumen 156 configured to threadedly receive the threaded post 162 of the lock screw 116 to enable secure coupling of the locking element 114 and the lock screw 116. In some embodiments, the locking element 114 is configured to radially flex as the lock screw 116 is advanced into the central opening 128 of the base 112 and the head 160 of the lock screw 116 exerts a compressive force against the top surface 150 of the locking element 114. In some embodiments, the locking element 114 may have a perimeter edge 158 configured to cooperate with the perimeter ridge 126 of the base 112 to capture or pinch the one or more tensionable fixation members 18 within a locking interface between the perimeter ridge 126 of the base 112 and the perimeter edge 158 upon radial flexing of the locking element 114 thereby providing a secondary locking feature.

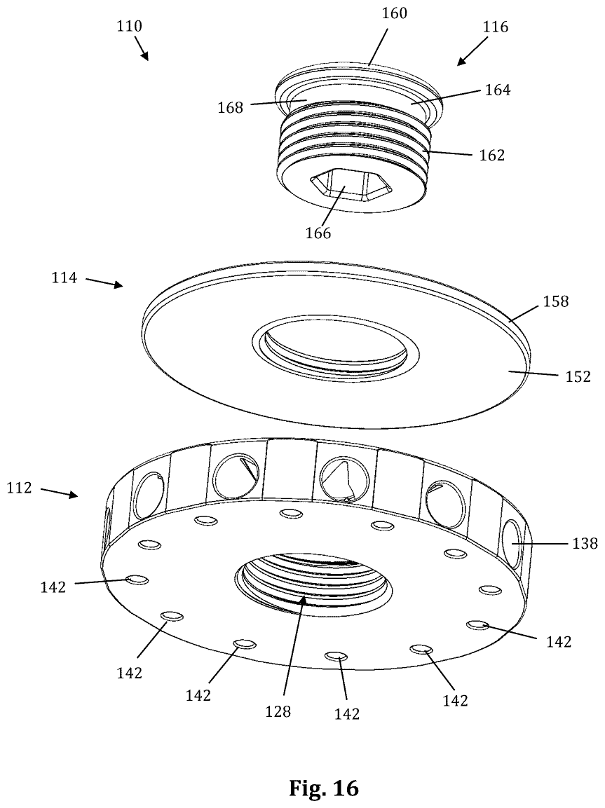

Referring again to FIGS. 15-16, the lock screw 116 may include a head 160, a threaded post 162 extending distally from the head 160, a neck recess 164 positioned between the head 160 and the threaded post 162, and a central lumen 166 extending through the lock screw 116 along the central axis. In some embodiments, the neck recess 164 extends circumferentially around the perimeter of the threaded post 162 and includes a smooth surface 168. In some embodiments, the locking element 114 is coupled to the lock screw 116 by threadedly advancing the lock screw 116 through the central opening 154 of the locking element 114. By way of example, the threaded post 162 may advance fully through the central opening 154 until the neck recess 164 is positioned within the threaded opening 154. At this point, the locking element 114 is captured by the neck recess 164, preventing dissociation of the locking element 114 from the lock screw 116 while allowing rotational movement of the lock screw 116 relative to the locking element 114 when the locking element 114 is positioned within the neck recess 164. This feature ensures that the lock screw 116 can rotate relative to the locking element 114 upon actuation of the lock screw 116, but the locking element 114 does not rotate against the tensionable fixation members 18 during final tightening of the locking element 114, which could introduce wear due to friction on the tensionable fixation members 18 as well as potentially adversely impact the tension applied to the tensionable fixation members 18. In some embodiments, the central lumen 166 is sized and shaped to facilitate engagement with a driver tool. For example, the central lumen 166 as shown by way of example only in FIGS. 15-16 has a hexagon-shaped perimeter shape, however shapes are possible. By way of example, the threaded post 162 is configured to threadedly engage the threaded surface 130 of the central recess 128 of the base 112. In some embodiments, actuation of the lock screw 116 (e.g., by a driver tool) advances the lock screw 116 into the central opening 128, which causes the head 160 of the lock screw 116 to exert a compressive force on the locking element 114, which in turn exerts a compressive force on the tensionable fixation members 18 within the locking interface described above.

In some embodiments, the sytstem 110 of the present disclosure may be used in soft tissue-to-bone and/or bone-to-bone repair. To use the sytstem 110, a user may first place the base 112 on a portion of a bone to which the damaged soft tissue and/or bone is to be attached (or attach the base 112 to a fixation plate or a prosthesis). The user may then opt to secure the placement on the bone (or plate or prosthesis) by inserting one or more purchase members (e.g., screws, wires, etc.) through one or more vertical openings 142 to ensure the base 112 remains in place during use. At this point, the user may begin the repair by attaching or otherwise associating a fixation end 18b of at least one tensionable fixation member 18 to the damaged soft tissue or bone. The free end 18a of the attached tensionable fixation member 18 may be associated with a chaperone member (e.g., needle, passing loop, etc.) and then passed through a lateral opening 138 of the base 112, for example a lateral opening 138 that is closest to and/or oriented toward the damaged tissue/bone to ensure the most efficient path to reduction. The free end 18a is then passed through the fixation channel 140 and cleat 132 associated with the selected lateral opening 138 such that the free end 18a extends proximally from the top surface 120 of the base 112. The use may then adjust the tension in the tensionable fixation member 18 to a desired level, at which point the user may then urge the tensionable fixation member 18 into the tapered extension of the cleat 132 while maintaining the desired tension in the tensionable fixation member 18 to provisionally lock the tensionable fixation member 18 to the base 112 by wedging or pinching the tensionable fixation member 18 within the cleat 132.

This process may be repeated multiple times with multiple tensionable fixation members 18 passing through distinct lateral openings 138/cleat 132 combinations. In some embodiments, the fixation ends 18b of the additional tensionable fixation members 18 may be attached to the same damaged tissue and/or bone and/or prosthesis or distinct damaged tissue and/or bone and/or prosthesis. Once all the tensionable fixation members 18 have been provisionally locked within their respective cleats 132, the user may then apply the secondary lock by coupling the locking element 114 to the base 112. Prior to coupling the locking element 114 to the base 112, the locking element 114 may by coupled to the lock screw 116 as described above such that the locking element 114 is positioned within the neck recess 164. In some embodiments, the locking element 114 and lock screw 116 may be provided in an already coupled state so the user does not have to perform this step themselves. The secondary locking step may be accomplished by aligning the threaded post 162 of the lock screw 116 with the central opening 128 of the base 112, and then actuating the lock screw 116 by rotationally advancing the lock screw 116 into the central opening 128 until the locking element 114 contacts the tensionable fixation members 18. Final tightening (e.g., the secondary lock) is then accomplished by further advancement of the lock screw 116. Once the locking element 114 is in contact with the tensionable fixation members 18, further rotation of the lock screw 116 will apply a compressive force on the locking element 114, causing the locking element 114 to flex radially outward which in turn causes the locking element 114 to compress the tensionable fixation members 18 without rotating. This will effectively pinch the tensionable fixation members 18 within the locking interface between the medial surface 146 of the perimeter ridge 126 of the base 112 and the perimeter edge 158 of the locking element 114. At this point the construct is secured and excess free ends 18a of the tensionable fixation members 18 may be removed.

If the user determines at any point that any of the tensionable fixation members 18 needs to be re-tensioned, the lock screw 116 may be rotated in the opposite direction (e.g., in a counterclockwise direction) which will release the locking element 114 from the tensionable fixation members 18, enabling the locking element 114 to be temporarily removed. Once this happens, the tensionable fixation members 18 should still be provisionally locked under the original applied tension by virtue of being wedged within their respective cleats 132. To adjust tension in any one tensionable fixation member 18, the user merely locates the tensionable fixation member 18 at issue, pulls the tensionable fixation member 18 toward the central opening 128 to decouple the tensionable fixation member 18 from the tapered extension 136 of the cleat 132, adjusts the tension to a desired level, and re-wedges the tensionable fixation member 18 within the tapered extension 136 of the cleat 132. This can be repeated independently for any tensionable fixation member 18 that needs adjusting. Once it has been determined that all the tensionable fixation members 18 have the desired amount of tension, the locking element 114 may be recoupled to the base 112 as described above.

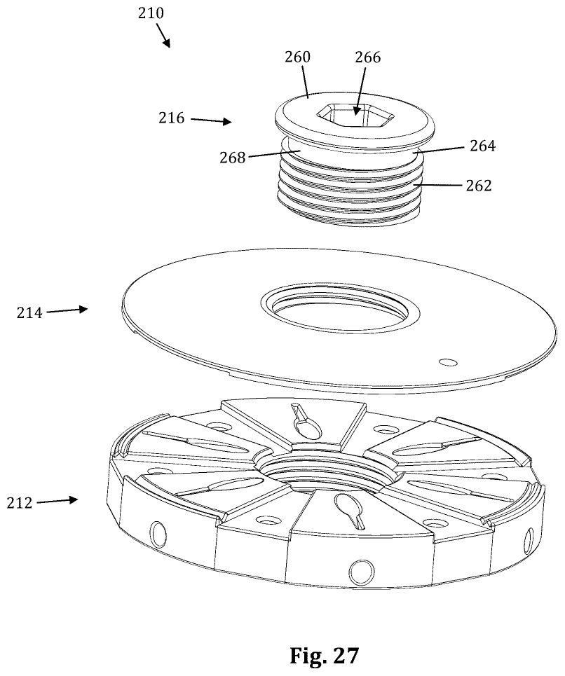

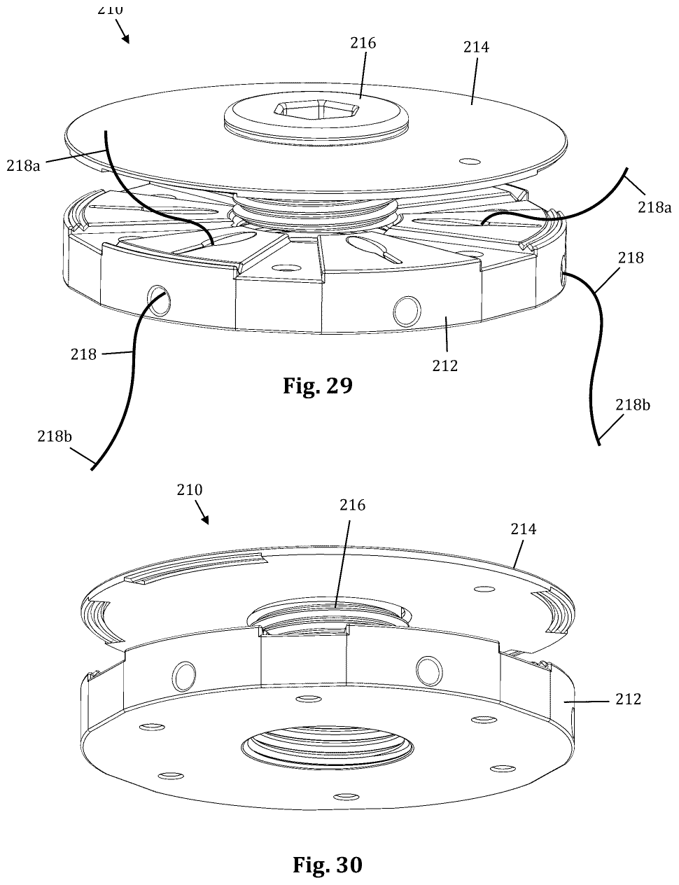

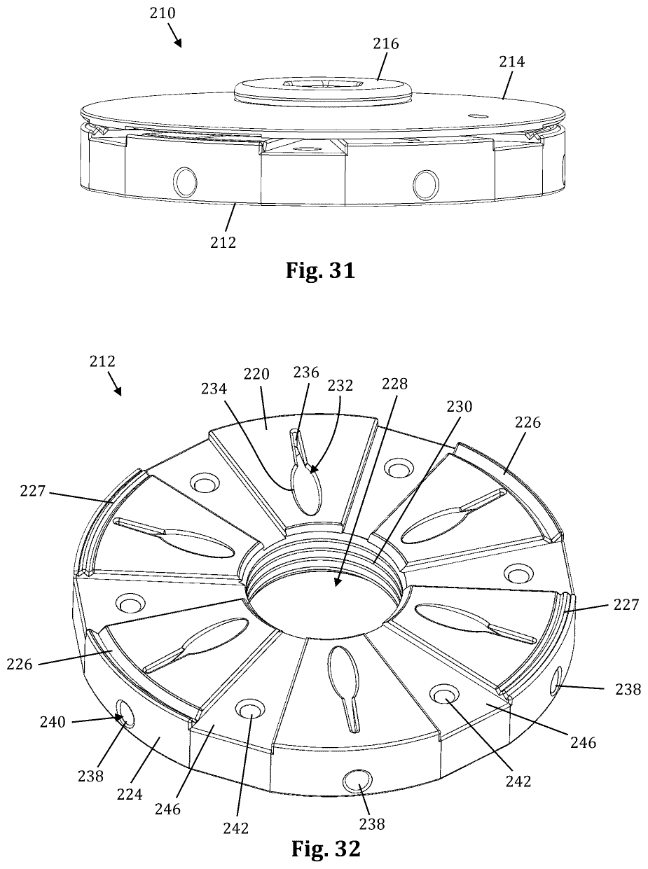

FIGS. 27-31 illustrate an example of a knotless tensionable fixation system 210 according to some embodiments of the disclosure. By way of example only, the knotless tensionable fixation system 210 (also referred to herein as “system 210”) includes a base 212, a locking element 214, a lock screw 216 and at least one tensionable fixation member 18. The system 210 of the present disclosure enables a user to achieve complex soft tissue-to-bone or bone-to-bone repair by first placing the base 212 against a first bone segment (or securing to a fixation plate or prosthesis) and then securing multiple tensionable fixation members 18 (e.g., surgical sutures, tape, wires, etc.) that may be attached to the same or different tissue or bone to be repaired (e.g., several unique tensionable fixation member strands may be attached the same tissue, or unique tensionable fixation member strands may be attached to different bone fragments or tissue segments), or to another artificial fixation member such as a prosthesis. In some embodiments, each tensionable fixation member 18 has one or more free ends 18a and a target engagement portion 18b, the target engagement portion 18b may be attached to a tissue or bone segment to be repaired or a surgical implant or prosthesis, and the tensionable fixation member 18 is passed through the base 212 such that the one or more free ends 18a extend proximally from the base 212 to enable manipulation by a user. In some embodiments, the target engagement portion 18b comprises one end of the tensionable fixation member, which then has one free end 18a. In some embodiments, the target engagement portion 18b may be a loop or middle portion of the tensionable fixation member 18, which then has two free ends 18a that may be secured by the base 212. In some embodiments, the system 210 enables each tensionable fixation member 18 to be provisionally secured to the base 212 under adequate tension while other tensionable fixation members 18 (or another strand of the same tensionable fixation member 18) are being utilized. In some embodiments, once all tensionable fixation members 18 have been provisionally secured to the base 212 under the desired tension to achieve reduction of the repair, the locking element 214 may be coupled to the base 212 and secured by the lock screw 216 under compression to provide a secondary or final lock (e.g., the provisional lock being a first lock) simultaneously to all tensionable fixation members 18 to securely lock all tensionable fixation members 18 under tension at the same time and with one single locking element 214.

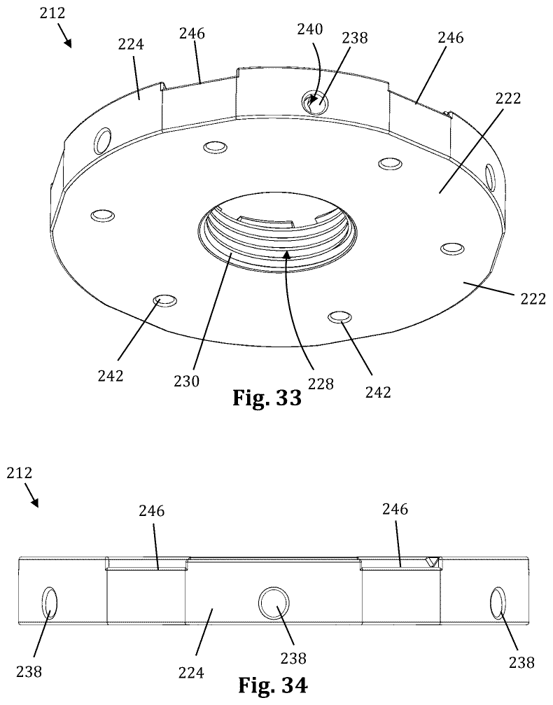

FIGS. 32-35 illustrate an example of a base 212 forming part of the system 210, according to some embodiments. By way of example, the base 212 may have any perimeter shape suitable to allow fixation of multiple tensionable fixation members 18 in multiple directions, including but not limited to circular (shown by way of example in FIGS. 27-35), semi-circular, curved, triangular, square, oval, hexagonal, octagonal, pentagonal, and the like. In some embodiments, the base 212 may have a top or proximal surface 220, a bottom or distal surface 222, and a lateral surface 224, which may be a planar surface or a circumferential surface as shown, depending on the perimeter shape of the base. In some embodiments, the base 212 may have one or more single perimeter recesses 226 configured to engage with a single perimeter ridge 258 of the locking element 214 when the locking element 214 is advanced onto the base 212. In some embodiments, the base 212 may have one or more double perimeter recesses 227 configured to engage with a double perimeter ridge 259 of the locking element 214 when the locking element 214 is advanced onto the base 212. In some embodiments, one or more tensionable fixation members 18 may be captured or pinched between the single perimeter recesses 226 and single perimeter ridges 258 and/or the double perimeter recesses 227 and the double perimeter ridges 259 thereby providing a secondary locking feature. In some embodiments, the single perimeter recesses 226 of the base 212 and single perimeter ridges 258 of the locking element 214, and/or the double perimeter recesses 227 of the base 212 and double perimeter ridges 259 of the locking element 214 cooperate to form locking interfaces configured to capture and compress the tensionable fixation members 18 therebetween upon actuation of the lock screw 216, thereby providing the secondary locking feature.

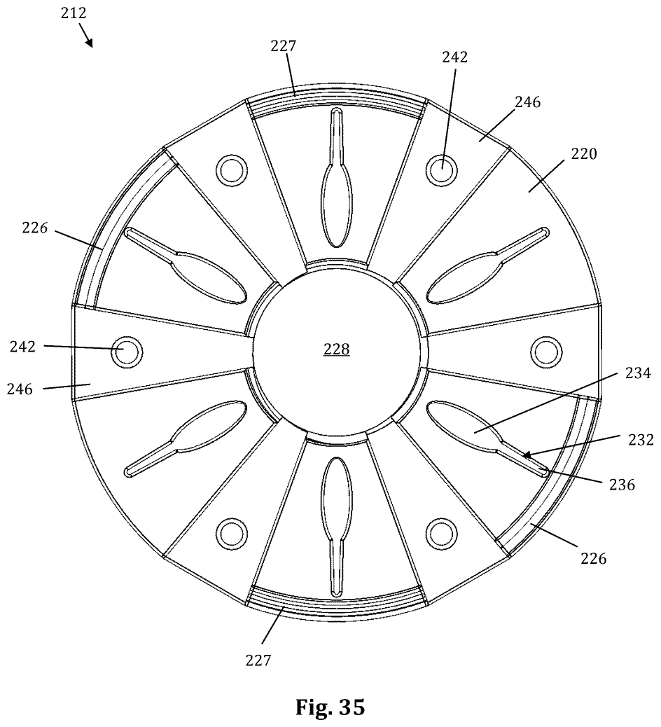

In some embodiments, the base 212 includes a central opening 228 extending through the base 212 from the top surface 220 to the bottom surface 222 along a central axis. In some embodiments, the central opening 228 includes a threaded lumen 230 configured to threadedly receive the threaded post 262 of the lock screw 216 to secure the lock screw 216 to the base 212. a plurality of cleats 232 distributed around the central opening 228. In some embodiments, the plurality of cleats 232 may be positioned equidistant from one another in an annular fashion, as shown by way of example in FIG. 35, however other configurations are possible. For example, in some embodiments, the plurality of cleats 232 may be grouped or bunched to one or more sides of the base 212. In some embodiments, each cleat 232 comprises an oblong opening 234 formed in the top surface 220 and a tapered extension 236. In some embodiments, the cleats 232 may be oriented such that the oblong ends 234 are positioned near the central opening 228 and the tapered extensions 236 are positioned near the outer perimeter of the base 212. In some embodiments, the tapered extensions 236 are wider near the oblong end 234 and narrower near the perimeter ridge 226.

In some embodiments, the base 212 includes a plurality of lateral openings 238 formed in the lateral surface 224 with each lateral opening 238 providing access to a fixation channel 240 extending diagonally (toward the top surface 220) into the base 212. In some embodiments, each lateral opening 238 is aligned with a unique cleat 232 such that the associated fixation channel 240 extends diagonally between the lateral opening 238 and the oblong opening 234 of the cleat 232, with the tapered extension 236 being open to the fixation channel 240. Thus, the base 212 is configured such that, after securing a fixation end 18b of a tensionable fixation member 18 to a tissue or bone segment to be repaired (or fixation plate or prosthesis), the free end 18a of the tensionable fixation member 18 may be passed (e.g., using a chaperone element such as a needle, passing loop, or guide wire, etc.) into the base 212 through the lateral opening 238, along the diagonal fixation channel 240, and emerging through the top surface 220 through the associated cleat 232, either by way of the oblong opening 234 or a wider portion of the tapered extension 236. After pulling on the free end 18a to apply a desired amount of tension to the tensionable fixation member 18, the user may then pull laterally on the tensionable fixation member 18 to urge the tensionable fixation member into (or farther into) the tapered extension 236 of the cleat 232 so that the tensionable fixation member 18 is wedged or otherwise captured by the tapered extension 236. This provides the primary or provisional locking of the tensionable fixation member 18 to the base 212 described herein.

In some embodiments, the base 212 further includes one or more vertical openings 242 extending through the base 212 from the top surface 220 to the bottom surface 222. By way of example, the vertical openings 242 may be configured to enable passage of a purchase element (not shown) configured to extend through the vertical opening 242 and into the bone segment (or fixation plate or prosthesis) that the base 212 is positioned against in order to secure the base 212 to the bone prior to performing the repair procedure (or after commencement of the repair procedure if determined to be necessary). In some embodiments, the purchase element may be (by way of example only) a bone screw, pin, wire, tack, or any member capable of securing the base 212 to a bone. In some embodiments, the base 212 includes a plurality of vertical openings 242. In some embodiments, the plurality of vertical openings 242 may be interspersed between the cleats 232. In some embodiments, the number of vertical openings 242 may be less than the number of cleats 232. In some embodiments, the vertical openings 242 may be positioned within recesses 246 formed in the top surface 220. In some embodiments, the vertical openings 242 may be configured to enable passage of one or more tensionable fixation members 18 therethrough.

In some embodiments, the bottom surface 222 may be planar. In some embodiments, the bottom surface 222 may be contoured to more effectively rest against bone or a fixation plate or a prosthesis. In some embodiments, the bottom surface 222 may be concave.

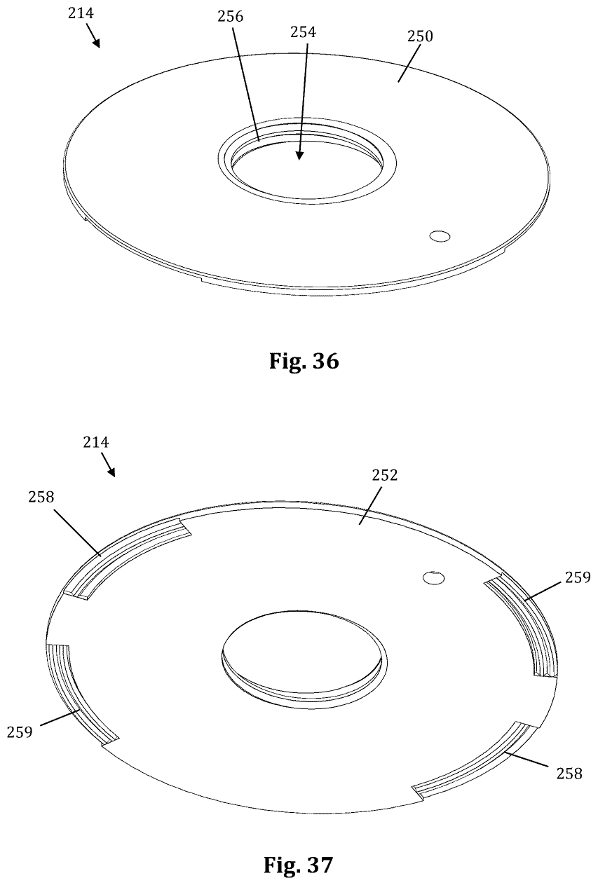

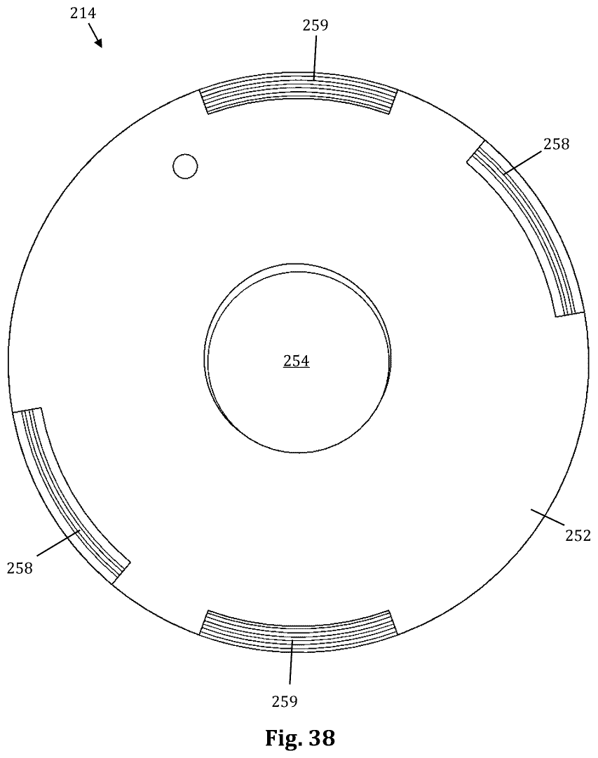

FIGS. 36-38 illustrate an example of a locking element 214, according to some embodiments. In some embodiments, the locking element 214 has an outer perimeter shape corresponding to the perimeter shape of the base 212. In some embodiments, the locking element 214 may be sized to fit on top of the base 212 such that the outer perimeter dimensions of the locking element 214 are substantially similar to the outer perimeter dimensions of the base 212. In some embodiments, the locking element 214 has a proximal or top surface 250, a distal or bottom surface 252, and a central opening 254 extending through the locking element 214 from the top surface 250 to the bottom surface 252 along the central axis. In some embodiments, the bottom surface 252 may be concave. In some embodiments, the central opening 254 includes a threaded lumen 256 configured to threadedly receive the threaded post 262 of the lock screw 216 to enable secure coupling of the locking element 214 and the lock screw 216. In some embodiments, the locking element 214 may have one or more single perimeter ridges 258 configured to mate with the single perimeter recesses 226 of the base 212, and one or more double perimeter ridges 259 configured to mate with the double perimeter recesses 227 of the base 212 to capture or pinch the one or more tensionable fixation members 18 within locking interfaces between the single perimeter recesses 226/ridges 258 and double perimeter recesses 227/ridges 259 upon application of the locking element 114 and actuation of the lock screw 116 thereby providing a secondary locking feature.

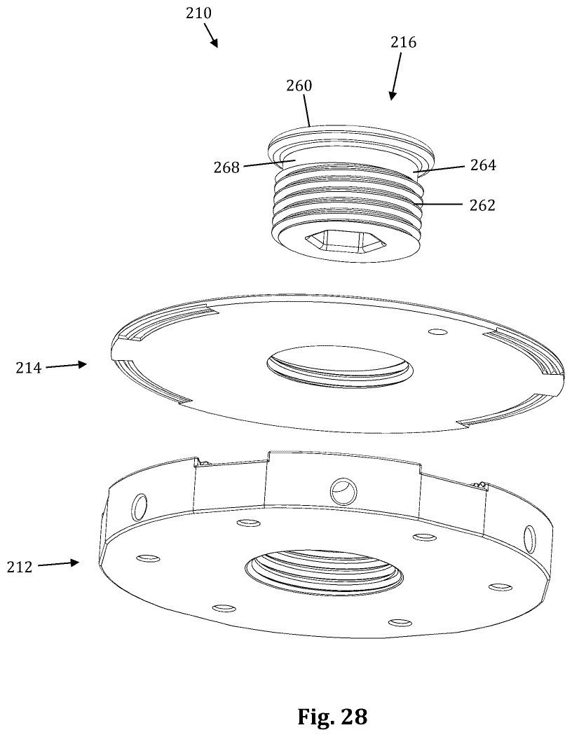

Referring again to FIGS. 27-28, the lock screw 216 may include a head 260, a threaded post 262 extending distally from the head 260, a neck recess 264 positioned between the head 260 and the threaded post 262, and a central lumen 266 extending through the lock screw 216 along the central axis. In some embodiments, the neck recess 264 extends circumferentially around the perimeter of the threaded post 262 and includes a smooth surface 268. In some embodiments, the locking element 214 is coupled to the lock screw 216 by threadedly advancing the lock screw 216 through the central opening 254 of the locking element 214. By way of example, the threaded post 262 may advance fully through the central opening 254 until the neck recess 264 is positioned within the threaded opening 254. At this point, the locking element 214 is captured by the neck recess 264, preventing dissociation of the locking element 214 from the lock screw 216 while allowing rotational movement of the lock screw 216 relative to the locking element 214 when the locking element 214 is positioned within the neck recess 264. This feature ensures that the locking element 214 does not rotate against the tensionable fixation members 18 when the lock screw 216 is actuated during final tightening of the locking element 214, which could introduce wear due to friction on the tensionable fixation members 18 as well as potentially adversely impact the tension applied to the tensionable fixation members 18. In some embodiments, the central lumen 266 is sized and shaped to facilitate engagement with a driver tool. For example, the central lumen 266 as shown by way of example only in FIGS. 27-28 has a hexagon-shaped perimeter shape, however shapes are possible.

In some embodiments, the system 210 of the present disclosure may be used in soft tissue-to-bone and/or bone-to-bone repair. To use the system 210, a user may first place the base 212 on a portion of a bone to which the damaged soft tissue and/or bone is to be attached (or attach the base 212 to a fixation plate or prosthesis). The user may then opt to secure the placement on the bone (or fixation plate or prosthesis) by inserting one or more purchase members (e.g., screws, wires, etc.) through one or more vertical openings 242 to ensure the base 212 remains in place during use. At this point, the user may begin the repair by attaching (or otherwise associating) a fixation end 18b of at least one tensionable fixation member 18 to the damaged soft tissue or bone. The free end(s) 18a of the attached tensionable fixation member 18 may be associated with a chaperone member (e.g., needle, passing loop, etc.) and then passed through a lateral opening 238 of the base 212, for example a lateral opening 238 that is closest to and/or oriented toward the damaged tissue/bone to ensure the most efficient path to reduction. The free end 18a is then passed through the fixation channel 240 and cleat 232 associated with the selected lateral opening 238 such that the free end 18a extends proximally from the top surface 220 of the base 212. The user may then adjust the tension in the tensionable fixation member 18 to a desired level, at which point the user may then urge the tensionable fixation member 18 into the tapered extension of the cleat 232 while maintaining the desired tension in the tensionable fixation member 18 to provisionally lock the tensionable fixation member 18 to the base 212 by wedging or pinching the tensionable fixation member 18 within the cleat 232.

This process may be repeated multiple times with multiple tensionable fixation members 18 passing through distinct lateral openings 238/cleats 232. In some embodiments, the fixation ends 18b of the additional tensionable fixation members 18 may be attached to the same damaged tissue and/or bone and/or prosthesis or distinct damaged tissue and/or bone and/or prosthesis. Once all the tensionable fixation members 18 have been provisionally locked within their respective cleats 232, the user may then apply the secondary lock by coupling the locking element 214 to the base 212. Prior to coupling the locking element 214 to the base 212, the locking element 214 may by coupled to the lock screw 216 as described above such that the locking element 214 is positioned within the neck recess 264. In some embodiments, the locking element 214 and lock screw 216 may be provided in an already coupled state so the user does not have to perform this step themselves. The secondary locking step may be accomplished by aligning the threaded post 262 of the lock screw 216 with the central opening 228 of the base 212, and then rotationally advancing the lock screw 216 into the central opening 228 until the locking element 214 contacts the tensionable fixation members 18. Final tightening (e.g., the secondary lock) is then accomplished by further advancement of the lock screw 216. Once the locking element 214 is in contact with the tensionable fixation members 18, further rotation of the lock screw 216 will cause the locking element 214 to compress the tensionable fixation members 18 without rotating. This will effectively pinch the tensionable fixation members 18 within the locking interfaces between the single perimeter recesses 226 of the bases 212 and single perimeter ridges 258 of the locking element 214 and/or the double perimeter recesses 227 of the base 212 and the double perimeter ridges 259 of the locking element 214. At this point the construct is secured and excess free ends 18a of the tensionable fixation members 18 may be removed or tied.

If the user determines at any point that any of the tensionable fixation members 18 needs to be re-tensioned, the lock screw 216 may be rotated in the opposite direction (e.g., in a counterclockwise direction) which will release the locking element 214 from the tensionable fixation members 18, enabling the locking element 214 to be temporarily removed. Once this happens, the tensionable fixation members 18 should still be provisionally locked under the original applied tension by virtue of being wedged within their respective cleats 232. To adjust tension in any one tensionable fixation member 18, the user merely locates the tensionable fixation member at issue, pulls the tensionable fixation member 18 toward the central opening 228 to decouple the tensionable fixation member 18 from the tapered extension 236 of the cleat 232, adjusts the tension to a desired level, and re-wedges the tensionable fixation member 18 within the tapered extension 236 of the cleat 232. This can be repeated independently for any tensionable fixation member 18 that needs adjusting. Once it has been determined that all the tensionable fixation members 18 have the desired amount of tension, the locking element 214 may be recoupled to the base 212 as described above.

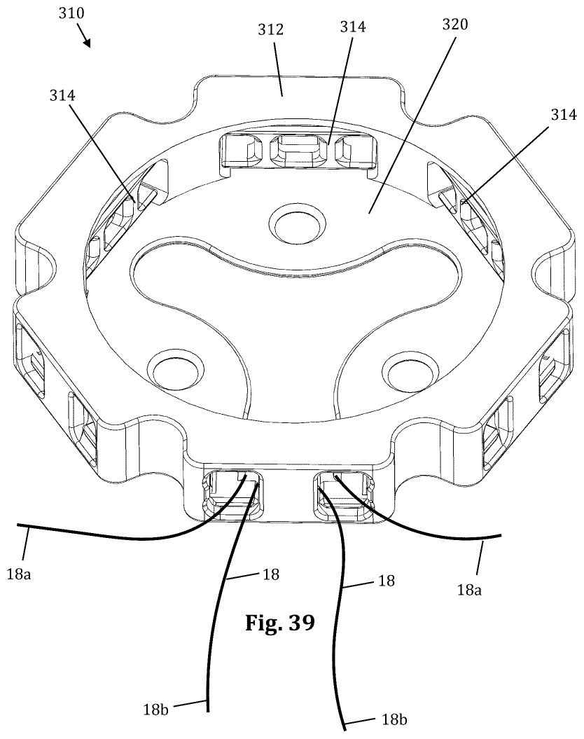

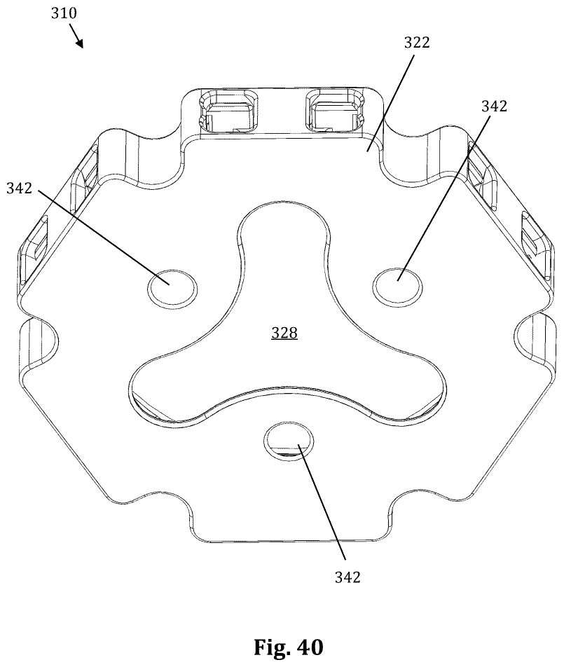

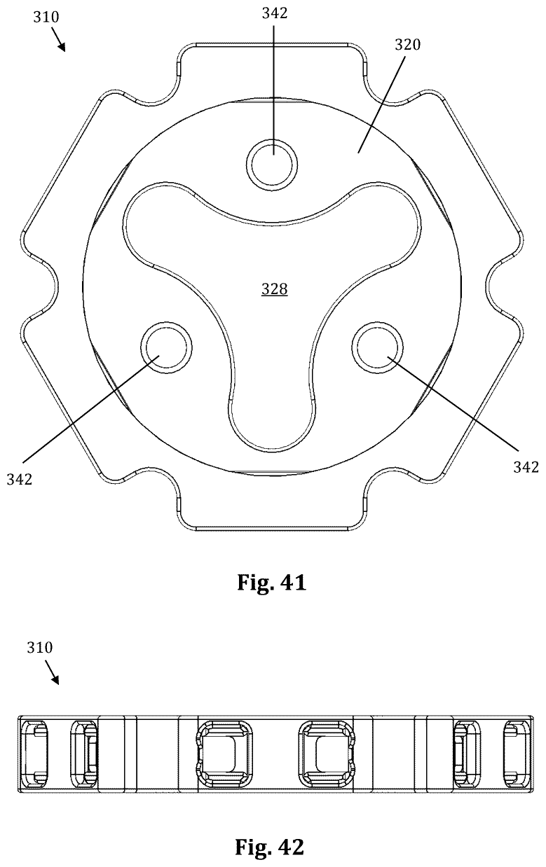

FIGS. 39-42 illustrate an example of a knotless tensionable fixation system 310 according to some embodiments of the disclosure. By way of example, the knotless tensionable fixation system 310 (also referred to herein as “system 310”) comprises a base 312 and a plurality of locking elements 314. By way of example, the system 310 is configured to enable independent tensionable fixation of multiple damaged tissue and/or bone by tethering the damaged tissue/bone to an undamaged bone using multiple tensionable fixation members 18. By way of example, in this embodiment, the tensionable fixation member 18 passes between the locking element 314 and the base 312 and is knotlessly secured by via pinching between locking surfaces on the locking element 314 and locking surfaces on the base 312, as will be explained.

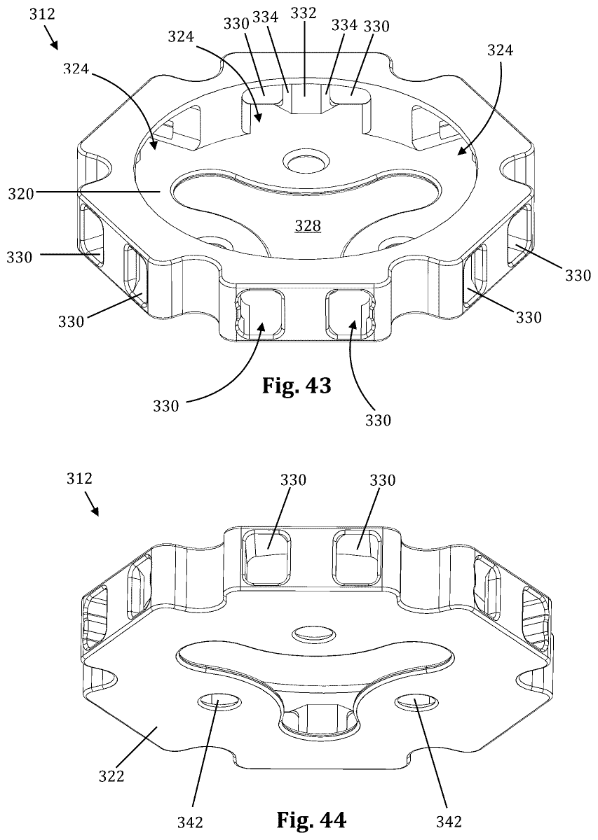

FIGS. 43-44 illustrate an example of a base 312 forming part of the system 310, according to some embodiments. By way of example, the base 312 may have any perimeter shape suitable to allow fixation of multiple tensionable fixation members 18 in multiple directions, including but not limited to circular (shown by way of example in the drawings), triangular, square, oval, hexagonal, octagonal, pentagonal, and the like. In some embodiments, the base 312 may have a top or proximal surface 320, a bottom or distal surface 322, and a plurality of lateral cavities 324, each lateral cavity 324 configured to receive a locking element 314 therein. In some embodiments, the bottom surface 322 may be planar. In some embodiments, the bottom surface 322 may be contoured to more effectively rest against bone, a fixation plate, or a prosthesis. In some embodiments, the bottom surface 322 may be concave.

In some embodiments, the base 312 includes central opening 328 extending through the base 312 from the top surface 320 to the bottom surface 322 along a central axis. In some embodiments, the base 312 further includes one or more vertical openings 342 extending through the base 312 from the top surface 320 to the bottom surface 322. By way of example, the vertical openings 342 may be configured to enable passage of a purchase element (not shown) configured to extend through the vertical opening 342 and into the bone segment that the base 312 is positioned against in order to secure the base 312 to the bone prior to performing the repair procedure (or after commencement of the repair procedure if determined to be necessary). In some embodiments, the purchase element may be (by way of example only) a bone screw, pin, wire, tack, or any member capable of securing the base 312 to a bone. In some embodiments, the base 312 includes a plurality of vertical openings 342. In some embodiments, each lateral cavity 324 includes a pair of lateral openings 330 separated by a crossbar 332. In some embodiments, the crossbar 332 includes a pair of sloped surfaces 334 which slope toward the lateral openings 330 and function as fixation surfaces during use.

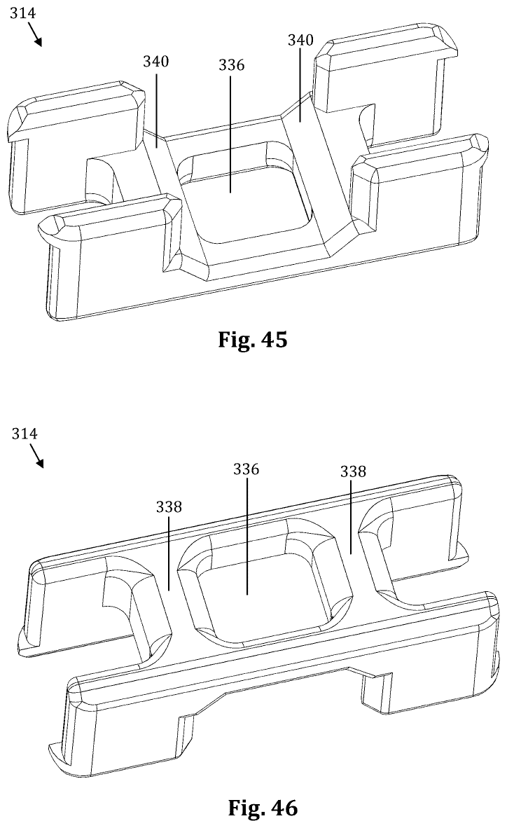

FIGS. 45-46 illustrate an example of a locking element 314 according to some embodiments. By way of example, the locking element may include a central opening 336 flanked by a pair of crossbars 338, each of which includes a sloped surface 340 sloping toward the central opening 336. In some embodiments, the sloped surfaces 340 of the locking element 314 aligns and cooperates with the sloped surfaces 334 of the lateral cavity 324 to capture the tensionable fixation member 18 therebetween to secure the tensionable fixation member 18 to the base 312 under tension.

By way of example, in use the base 312 may be secured to an undamaged bone segment (or fixation plate or prosthesis) by inserting one or more purchase members through one or more vertical openings 342. After a fixation end 18b is attached to a tissue or bone to be repaired, the free end 18a is chaperoned through one of the lateral openings 330, around one of the locking element crossbars 338, through the central opening 336 of the locking element 314, between the opposing sloped surfaces 334, 340 of the base 312 and locking element 314, respectively, and then back out through the original lateral cavity 324. Pulling on the tensionable fixation member 18 free end 18a not only tensions the tensionable fixation member 18 but also cinches the locking element 314 onto the base 312 capturing the tensionable fixation member 18 as described.

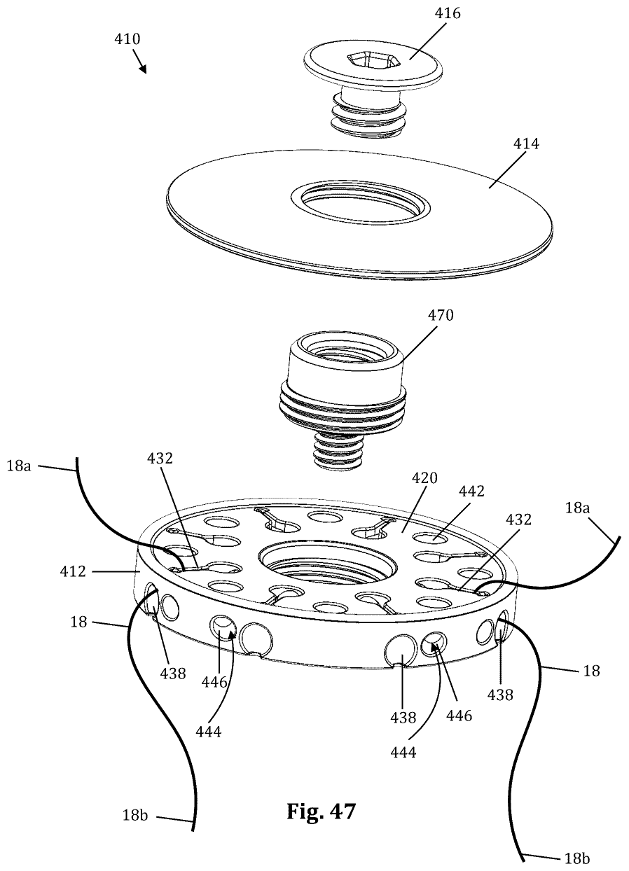

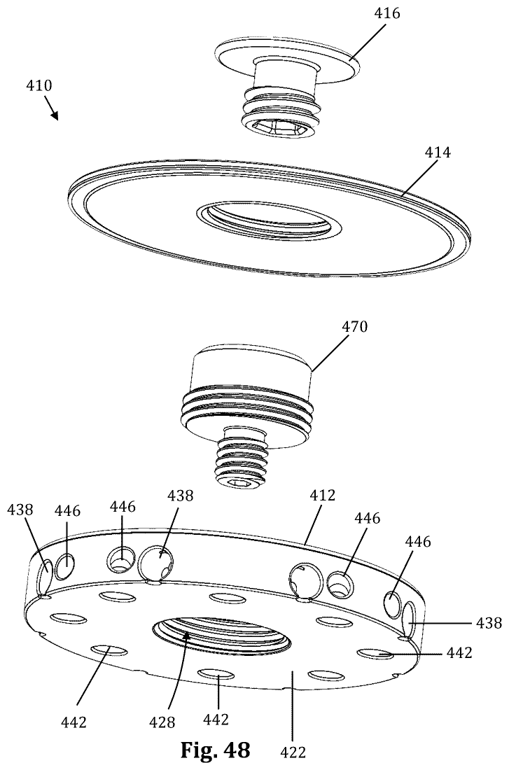

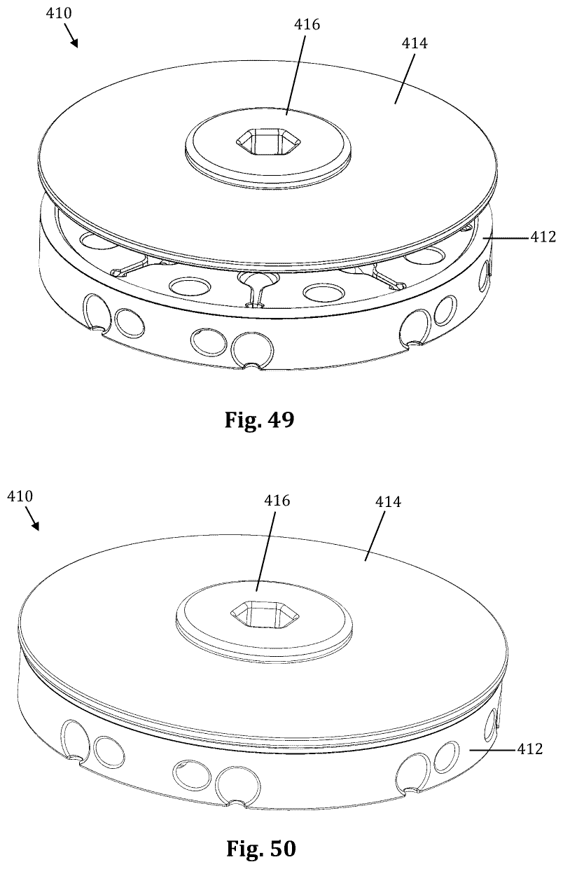

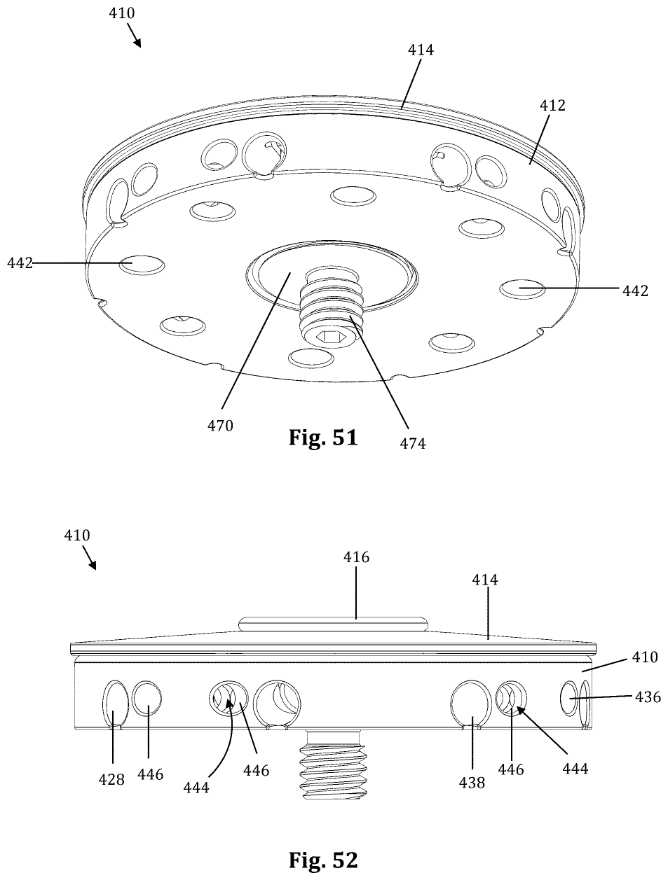

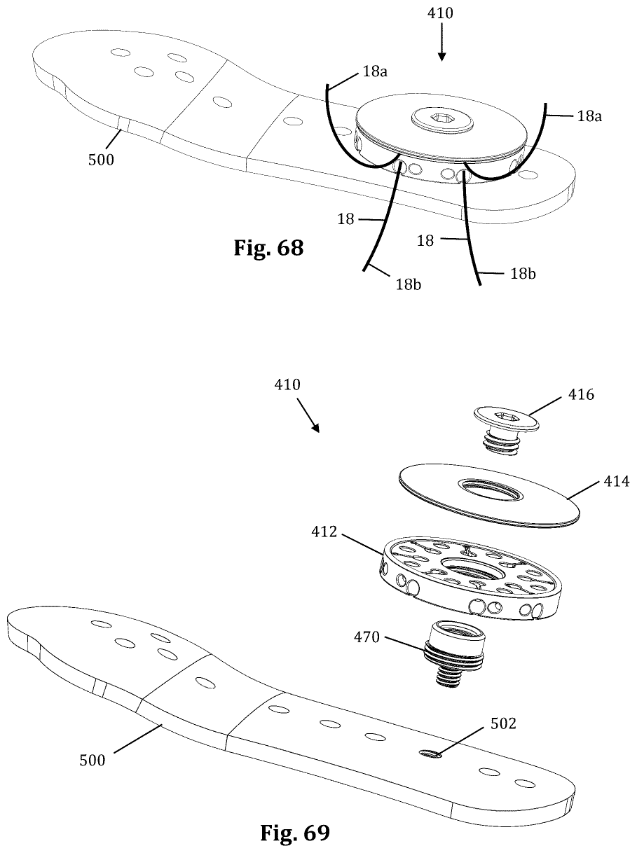

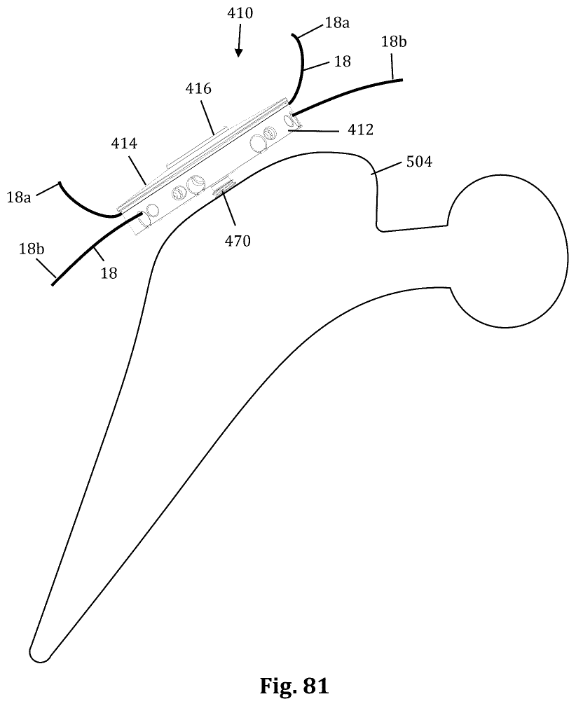

FIGS. 47-52 illustrate an example of a knotless tensionable fixation system 410 according to some embodiments of the disclosure. By way of example only, the knotless tensionable fixation system 410 (also referred to herein as “system 410”) includes a base 412, a locking element 414, a lock screw 416, at least one tensionable fixation member 18, and a coupling element 470. The system 410 of the present disclosure enables a user to achieve complex soft tissue-to-bone or bone-to-bone repair by first securing the base 412 to a first bone segment, an implanted fixation plate (e.g., FIGS. 68-71), or prosthesis (e.g., FIG. 81) and then securing multiple tensionable fixation members 18 (e.g., surgical sutures, tape, wires, etc.) that may be attached to the same or different tissue or bone to be repaired (e.g., several unique tensionable fixation member strands may be attached the same tissue, or unique tensionable fixation member strands may be attached to different bone fragments or tissue segments), or to another artificial fixation member such as a prosthesis. In some embodiments, each tensionable fixation member 18 has one or more free ends 18a and a target engagement portion 18b, the target engagement portion 18b may be attached to a tissue or bone segment to be repaired or a surgical implant or prosthesis, and the tensionable fixation member 18 is passed through the base 412 such that the one or more free ends 18a extend proximally from the base 412 to enable manipulation by a user. In some embodiments, the target engagement portion 18b comprises one end of the tensionable fixation member, which then has one free end 18a. In some embodiments, the target engagement portion 18b may be a loop or middle portion of the tensionable fixation member 18, which then has two free ends 18a that may be secured by the base 412. In some embodiments, the system 410 enables each tensionable fixation member 18 to be provisionally secured to the base 412 under adequate tension while other tensionable fixation members 18 (or another strand of the same tensionable fixation member 18) are being utilized. In some embodiments, once all tensionable fixation members 18 have been provisionally secured to the base 412 under the desired tension to achieve reduction of the repair, the locking element 414 may be coupled to the base 412 and secured by the lock screw 416 under compression to provide a secondary or final lock (e.g., the provisional lock being a first lock) simultaneously to all tensionable fixation members 18 to securely lock all tensionable fixation members 18 under tension at the same time and with one single locking element 414.

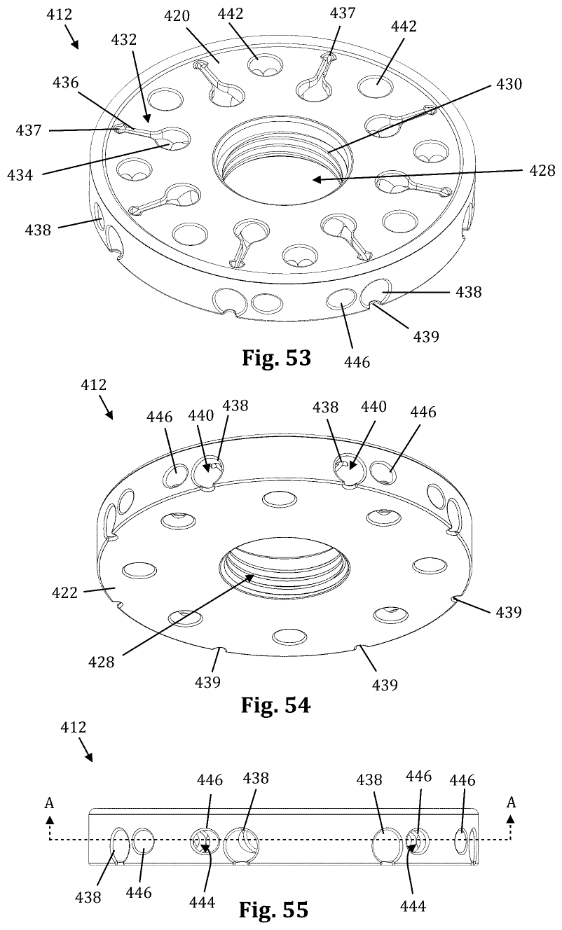

FIGS. 53-57 illustrate an example of a base 412 forming part of the system 410, according to some embodiments. By way of example, the base 412 may have any perimeter shape suitable to allow fixation of multiple tensionable fixation members 18 in multiple directions, including but not limited to circular (shown by way of example in FIGS. 47-57), semi-circular, curved, triangular, square, oval, hexagonal, octagonal, pentagonal, and the like. In some embodiments, the base 412 may have a top or proximal surface 420, a bottom or distal surface 422, and a lateral surface 424, which may be a planar surface, or a circumferential surface as shown, depending on the perimeter shape of the base. In some embodiments, the base 412 may have a perimeter ridge or lip 426 configured to engage with the perimeter recess 458 of the locking element 414 when the locking element 414 is advanced onto the base 412. In some embodiments, the perimeter ridge 426 of the base 412 and perimeter recess 458 of the locking element 414 cooperate to form a “locking interface” configured to capture and compress the tensionable fixation members 18 therebetween upon actuation of the lock screw 416, thereby providing the secondary locking feature.

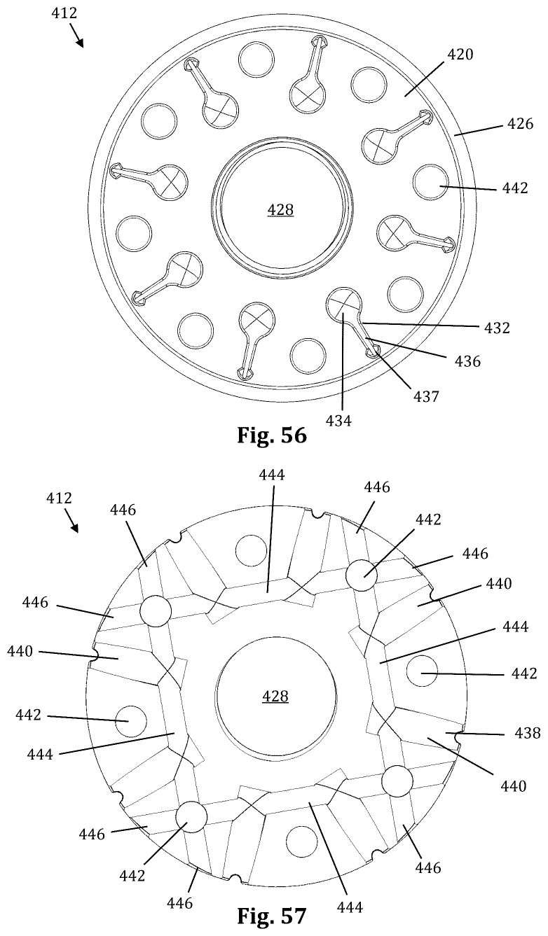

In some embodiments, the base 412 includes central opening 428 extending through the base 412 from the top surface 420 to the bottom surface 422 along a central axis. In some embodiments, the central opening 428 includes a threaded lumen 430 configured to threadedly receive the threaded distal portion 486 of the head 472 of the coupling element 470 (described below) to secure the coupling element 470 to the base 412. In some embodiments, the base 412 includes a plurality of cleats 432 formed in the top surface 420 distributed around the central opening 428. In some embodiments, the plurality of cleats 432 may be positioned equidistant from one another in an annular fashion, as shown by way of example in FIG. 56, however other configurations are possible. For example, in some embodiments, the plurality of cleats 432 may be grouped or bunched to one or more sides of the base 412. In some embodiments, each cleat 432 comprises a round opening 434 formed in the top surface 420 and a tapered extension 436. In some embodiments, the cleats 432 may be oriented such that the round openings 434 are positioned near the central opening 428 and the tapered extensions 436 extend toward the perimeter ridge 426. In some embodiments, the tapered extensions 436 are wider near the round opening 434 and narrower near the perimeter ridge 426. In some embodiments, the top surface 420 may include a plurality of sloped recesses 437 positioned at the radial ends of the tapered extensions 436 and configured to enable smooth passage of the tensionable fixation member 18 from the base 412.

In some embodiments, the base 412 includes a plurality of lateral openings 438 formed in the lateral surface 424 with each lateral opening 438 providing access to a fixation channel 440 extending diagonally (toward the top surface 420) into the base 412. In some embodiments, each lateral opening 438 is aligned with a unique cleat 432 such that the associated fixation channel 440 extends diagonally between the lateral opening 438 and the round opening 434 of the cleat 432, with the tapered extension 436 being open to the fixation channel 440. Thus, the base 412 is configured such that, after securing a fixation end 18b of a tensionable fixation member 18 to a tissue or bone segment to be repaired, the free end 18a of the tensionable fixation member 18 may be passed (e.g., using a chaperone element such as a needle, passing loop, or guide wire, etc.) into the base 412 through the lateral opening 438, along the diagonal fixation channel 440, and emerging through the top surface 420 through the associated cleat 432, either by way of the round opening 434 or a wider portion of the tapered extension 436. After pulling on the free end 18a to apply a desired amount of tension to the tensionable fixation member 18, the user may then pull laterally on the tensionable fixation member 18 to urge the tensionable fixation member into (or farther into) the tapered extension 436 of the cleat 432 so that the tensionable fixation member 18 is wedged or otherwise captured by the tapered extension 436. This provides a primary or provisional locking of the tensionable fixation member 18 to the base 412. In some embodiments, the base 412 may include one or more notches or recesses 439 at the intersection of one or more lateral openings 438 and the bottom surface 422, the recesses 439 being configured to improve clearance for certain types of needles during use. In some embodiments, the recesses 439 also enable use of a longer cleat 432 and/or fixation channel 440.

In some embodiments, the base 412 further includes one or more vertical openings 442 extending through the base 412 from the top surface 420 to the bottom surface 422. By way of example, the vertical openings 442 may be configured to enable passage of a purchase element (not shown) configured to extend through the vertical opening 442 and into the bone segment (or plate, prosthesis, etc.) that the base 412 is positioned against in order to secure the base 412 to the bone prior to performing the repair procedure (or after commencement of the repair procedure if determined to be necessary). In some embodiments, the purchase element may be (by way of example only) a bone screw, pin, wire, tack, or any member capable of securing the base 412 to a bone. In some embodiments, the base 412 includes a plurality of vertical openings 442. In some embodiments, the plurality of vertical openings 442 may be interspersed between the cleats 432. In some embodiments, the number of vertical openings 442 may be less than the number of cleats 432. In some embodiments, the vertical openings 442 may be used to pass additional tensionable fixation members 18 therethrough.

In some embodiments, the bottom surface 422 may be planar. In some embodiments, the bottom surface 422 may be contoured to more effectively rest against bone, plate, or prosthesis. In some embodiments, the bottom surface 422 may be concave.

In some embodiments, the base 412 may include a plurality of transverse channels 444 formed within the body 412 and extending between transverse openings 446 formed in the lateral surface 424, as best illustrated in FIG. 57. By way of example, the transverse channels 444 may configured to receive additional tensionable fixation members 18 or other fixation elements therein or therethrough, to enable a user to use additional fixation to secure the repair if desired. In some embodiments, the transverse channels 444 may be used to provide additional or alternative pathways for the tensionable fixation members 18 through the base 412. By way of example, the instant embodiment describes a plurality of transverse channels 444 provided in pairs of parallel channels positioned on either side of the central opening 428, with a first pair of parallel transverse channels 444 being perpendicular to a second pair of parallel transverse channels 444. However, any configuration of transverse channels 444 is possible so long as the transverse channels 444 do not extend through the central opening 428. In some embodiments, one or more transverse channels 444 may intersect with one or more vertical openings 442.

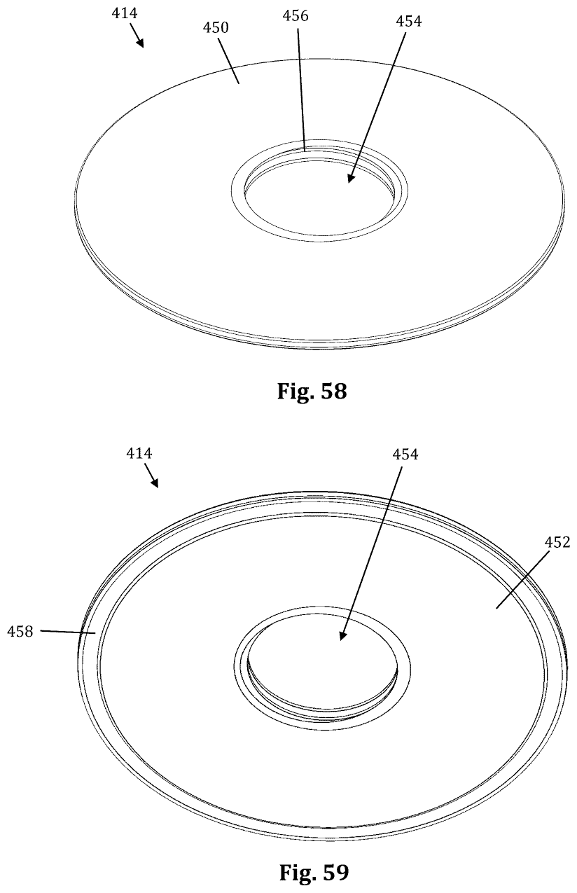

FIGS. 58-59 illustrate an example of a locking element 414, according to some embodiments. In some embodiments, the locking element 414 has an outer perimeter shape corresponding to the perimeter shape of the base 412. In some embodiments, the locking element 414 may be sized to fit on top of the base 412 such that the outer perimeter dimensions of the locking element 414 and base 412 are substantially similar. In some embodiments, the locking element 414 has a proximal or top surface 450, a distal or bottom surface 452, and a central opening 454 extending through the locking element 414 from the top surface 450 to the bottom surface 452 along the central axis. In some embodiments, the central opening 454 includes a threaded lumen 456 configured to threadedly receive the threaded post 462 of the lock screw 416 to enable secure coupling of the locking element 414 and the lock screw 416. In some embodiments, the locking element 414 may have a perimeter recess 458 configured to engage with the perimeter ridge 426 of the base 412 when the locking element 414 is advanced onto the base 412. In some embodiments, one or more tensionable fixation members 18 may be captured between the perimeter ridge 426 and perimeter recess 458 thereby providing a secondary locking feature.

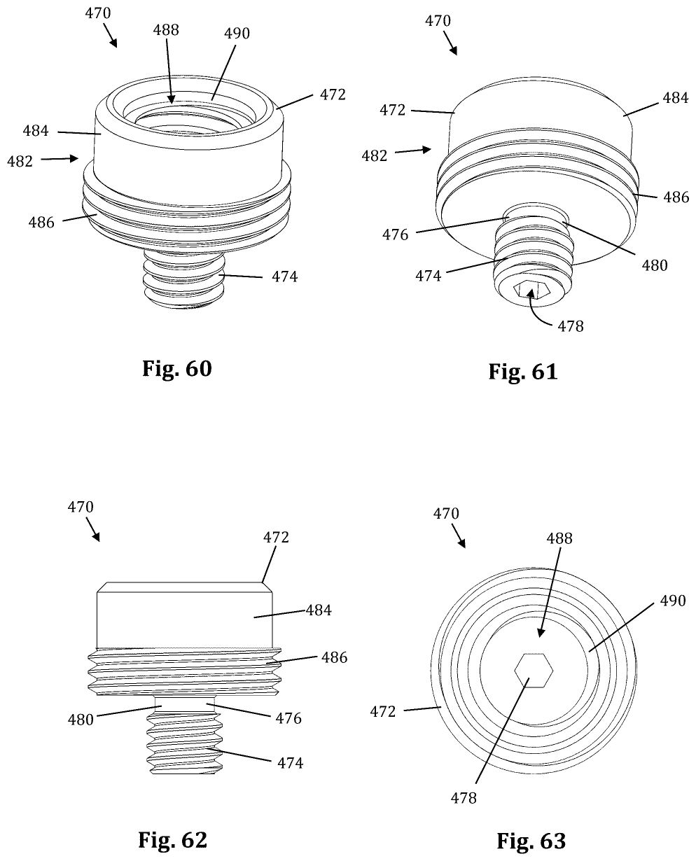

FIGS. 60-64 illustrate an example of a coupling element 470 forming part of the system 410, according to some embodiments. By way of example, the coupling element 470 may include a head 472, a threaded post 474 extending distally from the head 472, a neck recess 476 positioned between the head 472 and the threaded post 474, and a central lumen 478 extending through the coupling element 470 along the central axis. In some embodiments, the neck recess 476 extends circumferentially around the perimeter of the threaded post 474 and includes a smooth surface 480. In some embodiments, the coupling element 470 may be secured to a bone plate (e.g., the bone plate 500 shown by way of example only in FIGS. 68-69), a prosthesis, or a bone segment by way of the threaded post 474. In some embodiments, the threaded post 474 has a diameter value that is less than the diameter of the head 472. In some embodiments, the threaded post 474 is configured to engage a threaded through-hole 502 in plate 500 (FIGS. 68-69).

In some embodiments, the head 472 of the coupling element 470 has an outer surface 482 having a having a smooth proximal portion 484 and a threaded distal portion 486. In some embodiments, the head 472 further includes a proximal recess 488 having a threaded inner circumferential surface 490. By way of example, the proximal recess 488 is configured to threadedly receive the threaded post 462 of the lock screw 416 therein to secure the lock screw 416 to the coupling element 470 and by extension, the base 412. In some embodiments, the coupling element 470 may be coupled to the locking element 414 by threadedly advancing the coupling element 470 through the central opening 454 of the locking element 414. By way of example, the threaded portion 486 may advance fully through the central opening 454 until the smooth proximal portion 484 is positioned within the threaded opening 454. At this point, the locking element 414 is captured by the smooth proximal portion 484 and the head 460 of the lock screw 416 (which may already be coupled with the coupling element 470), preventing dissociation of the locking element 414 from the coupling element 470 while allowing rotational movement of the lock screw 416 relative to the locking element 414. This feature ensures that the locking element 414 does not rotate against the tensionable fixation members 18 during final tightening of the locking element 414, which could introduce wear due to friction on the tensionable fixation members 18 as well as potentially adversely impact the tension applied to the tensionable fixation members 18.

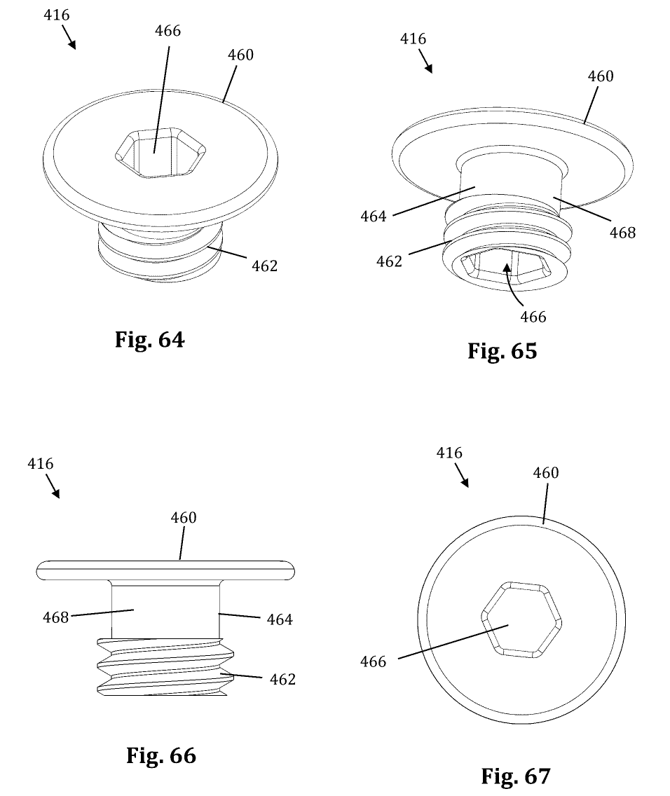

FIGS. 64-67 illustrate an example of a lock screw 416 forming part of the system 410, according to some embodiments. By way of example, the lock screw 416 may include a head 460, a threaded post 462 extending distally from the head 460, a neck recess 464 positioned between the head 460 and the threaded post 462, and a central lumen 466 extending through the lock screw 416 along the central axis. In some embodiments, the neck recess 464 extends circumferentially around the perimeter of the threaded post 462 and includes a smooth surface 468. In some embodiments, the lock screw 416 is coupled to the coupling element 470 by threadedly advancing the threaded post 462 of the lock screw 416 into the proximal recess 488 of the coupling element 470. By way of example, the head 460 has an outer perimeter that is configured to extend over and physically abut the top surface 450 of the locking element 414 during and after the final tightening. For example, during the final tightening process, the user will rotate the lock screw 416 to further advance the lock screw 416 into the proximal recess 488 of the coupling element 480. This will bring the head 460 into contact with the locking element 414 and further rotation of the lock screw 416 exerts a compressive force on the locking element 414 which in turn causes the locking element 414 to capture the tension members 18 within the locking interface between the perimeter recess 458 and the perimeter ridge 426 of the base 410. In some embodiments, the central lumen 466 is sized and shaped to facilitate engagement with a driver tool. For example, the central lumen 466 as shown by way of example only in FIGS. 64-67 has a hexagon-shaped perimeter shape, however shapes are possible.

In some embodiments, the system 410 of the present disclosure may be used in soft tissue-to-bone and/or bone-to-bone repair. To use the system 410, a user may first place the base 412 on a portion of a bone to which the damaged soft tissue and/or bone is to be attached. In some embodiments, the base 412 is coupled with a bone plate 500, for example as shown in FIGS. 68-71. At this point, the user may begin the repair by attaching a fixation end 18b of at least one tensionable fixation member 18 to the damaged soft tissue or bone. The free end 18a of the attached tensionable fixation member 18 may be associated with a chaperone member (e.g., needle, passing loop, etc.) and then passed through a lateral opening 438 of the base 412, for example a lateral opening 438 that is closest to and/or oriented toward the damaged tissue/bone to ensure the most efficient path to reduction. The free end 18a is then passed through the fixation channel 440 and cleat 432 associated with the selected lateral opening 438 such that the free end 18a extends proximally from the top surface 420 of the base 412. The user may then adjust the tension in the tensionable fixation member 18 to a desired level, at which point the user may then urge the tensionable fixation member 18 into the tapered extension of the cleat 432 while maintaining the desired tension in the tensionable fixation member 18 to provisionally lock the tensionable fixation member 18 to the base 412 by wedging or pinching the tensionable fixation member 18 within the cleat 432.

This process may be repeated multiple times with multiple tensionable fixation members 18 passing through distinct lateral openings 438/cleats 432. In some embodiments, the fixation ends 18b of the additional tensionable fixation members 18 may be attached to the same damaged tissue and/or bone and/or prosthesis or distinct damaged tissue and/or bone and/or prosthesis. Once all the tensionable fixation members 18 have been provisionally locked within their respective cleats 432, the user may then apply the secondary lock by coupling the locking element 414 to the base 412 by way of the coupling element 470 and lock screw 416. Prior to coupling the locking element 414 to the base 412, the locking element 414 may by coupled to the coupling element 470 and lock screw 416 as described above. In some embodiments, the locking element 414, coupling element 470, and lock screw 416 may be provided in an already coupled state so the user does not have to perform this step themselves. The secondary locking step may be accomplished by aligning the threaded post 462 of the lock screw 416 with the proximal recess 488 of the coupling element 470, and then rotationally advancing the lock screw 416 into the proximal recess 488 until the locking element 414 contacts the tensionable fixation members 18. Final tightening (e.g., the secondary lock) is then accomplished by further advancement of the lock screw 416. Once the locking element 414 is in contact with the tensionable fixation members 18, further rotation of the lock screw 416 will cause the locking element 414 to compress the tensionable fixation members 18 without rotating. This will effectively pinch the tensionable fixation members 18 within the locking interface between the perimeter ridge 426 of the base 412 and the perimeter recess 458 of the locking element 414. At this point the construct is secured and excess free ends 18a of the tensionable fixation members 18 may be removed.

If the user determines at any point that any of the tensionable fixation members 18 needs to be re-tensioned, the lock screw 416 may be rotated in the opposite direction (e.g., counterclockwise) which will release the locking element 414 from the tensionable fixation members 18, enabling the locking element 414 to be temporarily removed. Once this happens, the tensionable fixation members 18 should still be provisionally locked under the original applied tension by virtue of being wedged within their respective cleats 432. To adjust tension in any one tensionable fixation member 18, the user merely locates the tensionable fixation member at issue, pulls the tensionable fixation member 18 toward the central opening 428 to decouple the tensionable fixation member 18 from the tapered extension 436 of the cleat 432, adjust the tension to a desired level, and re-wedges the tensionable fixation member 18 within the tapered extension 436 of the cleat 432. This can be repeated independently for any tensionable fixation member 18 that needs adjusting.

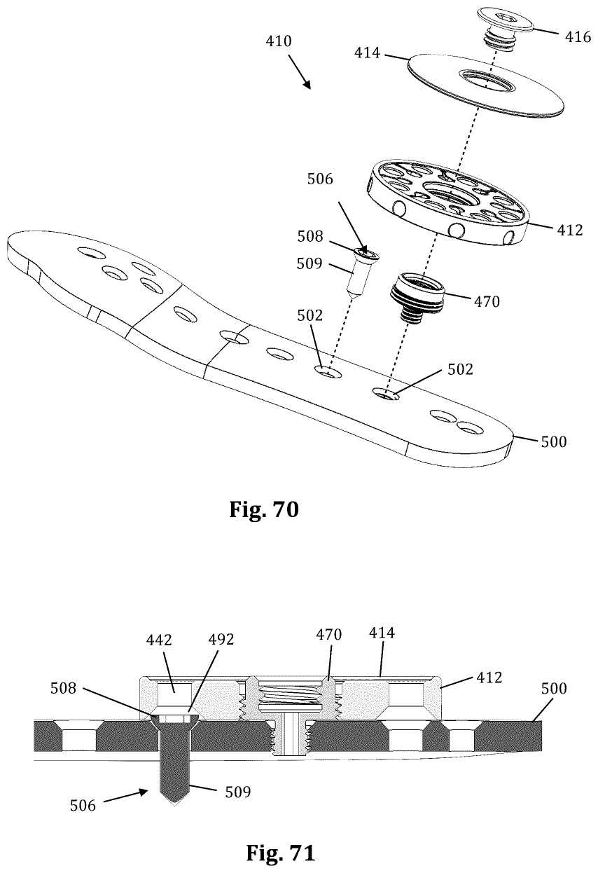

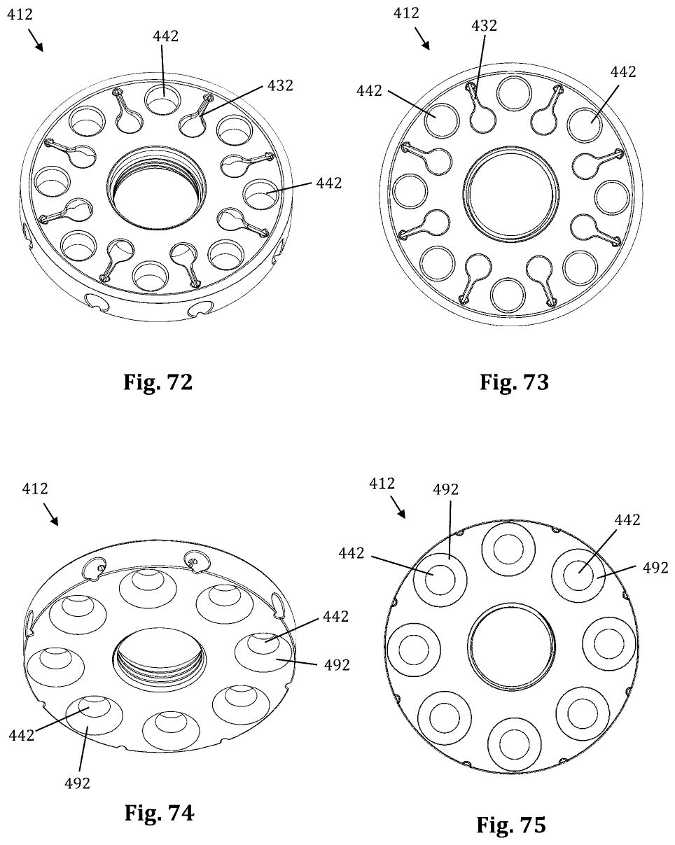

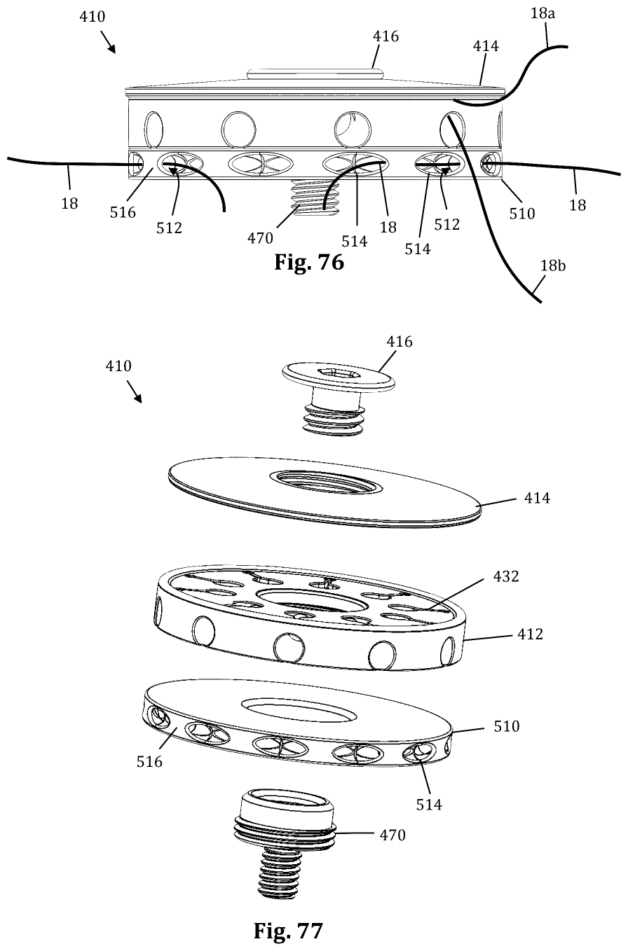

As previously mentioned, in some embodiments, the system 410 may be secured to a fracture fixation plate 500, as shown by way of example in FIG. 68. In some embodiments, the fracture fixation plate 500 may be secured to the bone fracture using one or more bone screws 506, each having a head 508 and a shank 509. By way of example, in some fracture plate systems the head 508 of the bone screw 506 may protrude beyond the surface profile of the fixation plate 500. In such cases the raised profile of the screw head 508 may interfere with the base 412. By way of example, FIGS. 70-75 illustrate an example of a system 410 coupled with a fixation plate 500 with a bone anchor 506 and having a base 412 including a plurality of recesses 492 on the bottom surface 422 of the base 412. In some embodiments, each recess 492 is positioned such that it surrounds a vertical opening 442 as shown by way of example in FIGS. 72-75. In some embodiments, each recess 492 is configured to receive the protruding profile of the head 508 of the bone screw 506 therein, as shown by way of example in FIG. 71. As such, the base 412 is able be positioned on the fracture fixation plate 500 without interference from the bone anchor 506.

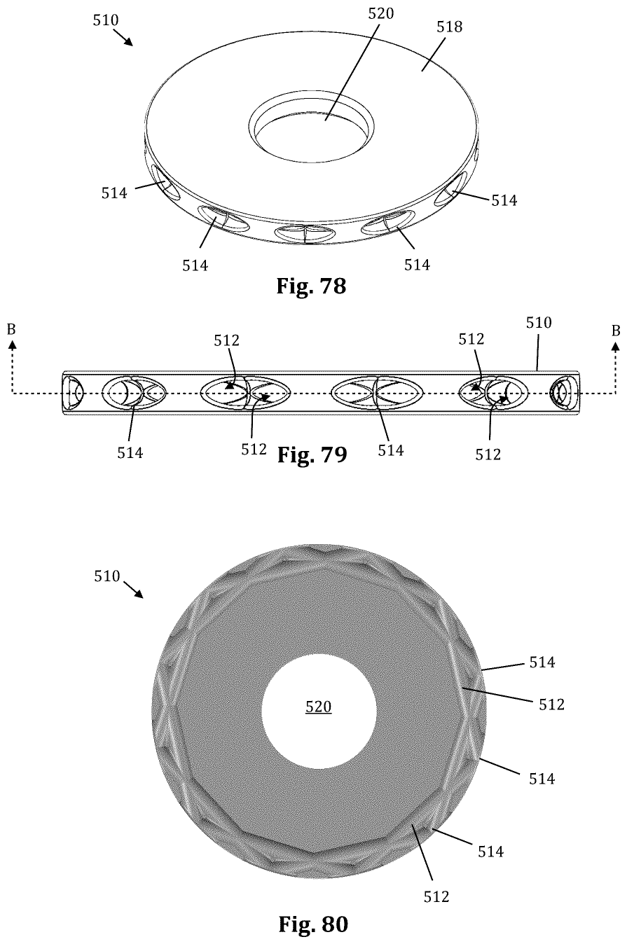

In some embodiments, the system 410 (or other described embodiments including system 10, system 110, system 210, system 610, system 710, and system 810) may further include a secondary tunnel plate 510, as shown by way of example only in FIGS. 76-80. In some embodiments, the secondary tunnel plate 510 may include a plurality of transverse channels 512 formed within the plate 510 and extending between transverse openings 514 formed in a lateral surface 516. By way of example, the transverse channels 512 may be configured to receive additional tensionable fixation members 18 or other fixation elements therein or therethrough, to enable a user to use additional fixation to secure the repair if desired. In some embodiments, the transverse channels 512 may be used to provide additional or alternative pathways for the tensionable fixation members 18 through the tunnel plate 510 and/or base 412, for example to enable a user to route a tensionable fixation member 18 to a cleat 432 on a different part of the base 412. In some embodiments, the tunnel plate 510 may include a generally planar top surface 518 and a central opening 520. In some embodiments, the central opening 520 is sized and configured to enable passage of the lock screw 416 and/or coupling element 470 therethrough.

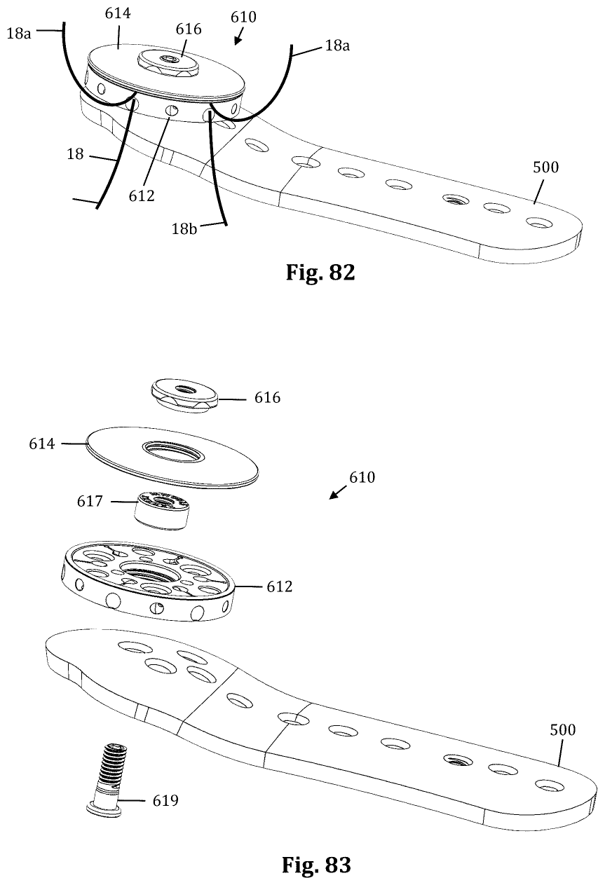

FIGS. 82-84 illustrate an example of a knotless tensionable fixation system 610 according to some embodiments of the disclosure. By way of example only, the knotless tensionable fixation system 610 (also referred to herein as “system 610”) includes a base 612, a locking element 614, a lock screw 616, a holder 617, a tensionable fixation member 18, and a plate coupler 619. In some embodiments, the system 610 is configured for coupling with a bone fixation plate 500, as shown by way of example in FIGS. 82-83. By way of example, the system 610 of the present disclosure enables a user to achieve complex soft tissue-to-bone or bone-to-bone repair by first securing the base 612 to a fixation plate 500 and then securing multiple tensionable fixation members 18 (e.g., surgical sutures, tapes, wires, etc.) that may be attached to the same or different tissue or bone to be repaired (e.g., several unique tensionable fixation member strands may be attached the same tissue, or unique tensionable fixation member strands may be attached to different bone fragments or tissue segments), or to another artificial fixation member such as a prosthesis. In some embodiments, each tensionable fixation member 18 has one or more free ends 18a and a target engagement portion 18b, the target engagement portion 18b may be attached to a tissue or bone segment to be repaired or a surgical implant or prosthesis, and the tensionable fixation member 18 is passed through the base 612 such that the one or more free ends 18a extend proximally from the base 612 to enable manipulation by a user. In some embodiments, the target engagement portion 18b comprises one end of the tensionable fixation member, which then has one free end 18a. In some embodiments, the target engagement portion 18b may be a loop or middle portion of the tensionable fixation member 18, which then has two free ends 18a that may be secured by the base 612. In some embodiments, the system 610 enables each tensionable fixation member 18 to be provisionally secured to the base 612 under adequate tension while other tensionable fixation members 18 (or another strand of the same tensionable fixation member 18) are being utilized. In some embodiments, once all tensionable fixation members 18 have been provisionally secured to the base 612 under the desired tension to achieve reduction of the repair, the locking element 614 may be coupled to the base 612 and secured by the lock screw 616 under compression to provide a secondary or final lock (e.g., the provisional lock being a first lock) simultaneously to all tensionable fixation members 18 to securely lock all tensionable fixation members 18 under tension at the same time and with one single locking element 614.

By way of example, the base 612 and locking element 614 are substantially similar to the base 12 and locking element 14 described above, in both form and function, and thus the description of the features of base 12 and locking element 14 apply to base 612 and locking element 614. By way of example, the base 612 may have any perimeter shape suitable to allow fixation of multiple tensionable fixation members 18 in multiple directions, including but not limited to circular (shown by way of example in FIGS. 82-84), semi-circular, curved, triangular, square, oval, hexagonal, octagonal, pentagonal, and the like. In some embodiments, the base 612 may have a perimeter ridge or lip 626 configured to engage with a perimeter recess (not shown but identical to perimeter recess 58 described above) of the locking element 614 when the locking element 614 is advanced onto the base 612. In some embodiments, the perimeter ridge 626 of the base 612 and perimeter recess of the locking element 614 cooperate to form a “locking interface” configured to capture and compress the tensionable fixation members 18 therebetween upon actuation of the lock screw 616, thereby providing the secondary locking feature.

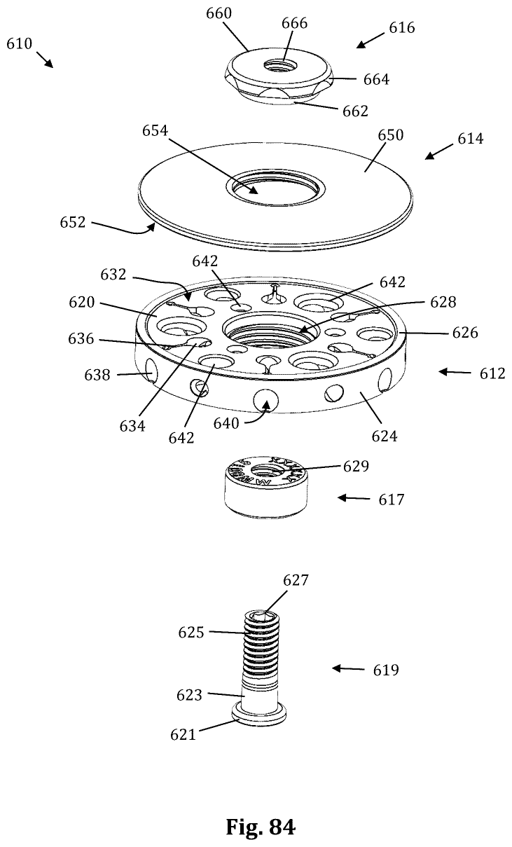

In some embodiments, the base 612 includes central opening 628 extending through the base 612, configured to receive the holder 617 therein. In some embodiments, the base 612 further comprises a plurality of provisional locking cleats 632 (hereinafter “cleats 632”) distributed around the central opening 628, which are identical in form and function to cleats 32 described above. In some embodiments, the plurality of cleats 632 may be positioned equidistant from one another in an annular fashion, as shown by way of example in FIG. 84, however other configurations are possible. For example, in some embodiments, the plurality of cleats 632 may be grouped or bunched to one or more sides of the base 612. In some embodiments, each cleat 632 comprises a round opening 634 formed in the top surface 620 and a tapered extension 636. In some embodiments, the cleats 632 may be oriented such that the round openings 634 are positioned near the central opening 628 and the tapered extensions 636 extend toward the perimeter ridge 626. In some embodiments, the tapered extensions 636 are wider near the round opening 634 and narrower near the perimeter ridge 626.

In some embodiments, the base 612 includes a plurality of lateral openings 638 formed in the lateral surface 624 with each lateral opening 638 providing access to a fixation channel 640 extending diagonally (toward the top surface 620) into the base 612. In some embodiments, each lateral opening 638 is aligned with a unique cleat 632 such that the associated fixation channel 640 extends diagonally between the lateral opening 638 and the round opening 634 of the cleat 632, with the tapered extension 636 being open to the fixation channel 640. Thus, the base 612 is configured such that, after securing a fixation end (or portion) 18b of a tensionable fixation member 18 to a tissue or bone segment to be repaired, the free end(s) 18a of the tensionable fixation member 18 may be passed (e.g., using a chaperone element such as a needle, passing loop, or guide wire, etc.) into the base 612 through the lateral opening 638, along the diagonal fixation channel 640, and emerging through the top surface 620 through the associated cleat 632, either by way of the round opening 634 or a wider portion of the tapered extension 636. After pulling on the free end 18a to apply a desired amount of tension to the tensionable fixation member 18, the user may then pull laterally on the tensionable fixation member 18 to urge the tensionable fixation member into (or farther into) the tapered extension 636 of the cleat 632 so that the tensionable fixation member 18 is wedged or otherwise captured by the tapered extension 636. This provides the primary or provisional locking of the tensionable fixation member 18 to the base 612 referred to above.

In some embodiments, the base 612 further includes one or more vertical openings 642 extending through the base 612. By way of example, the vertical openings 642 may be configured to enable passage of a purchase element (not shown) configured to extend through the vertical opening 642 and into fixation plate 500 that the base 612 is positioned against in order to secure the base 612 to the bone prior to performing the repair procedure (or after commencement of the repair procedure if determined to be necessary). In some embodiments, the base 612 includes a plurality of vertical openings 642. In some embodiments, the plurality of vertical openings 642 may be interspersed between the cleats 632. In some embodiments, the number of vertical openings 642 may be less than the number of cleats 632. In some embodiments, individual vertical openings of the plurality of vertical openings 642 may have different sizes and shapes. In some embodiments, one or more of the plurality of vertical openings 642 may be used to pass additional tensionable fixation members 18 therethrough.

By way of example, the locking element 614 is identical in form and function to the locking element 14 described above and thus the description of locking element 14 applies locking element 614. In some embodiments, the locking element 614 has an outer perimeter shape corresponding to the perimeter shape of the base 612. In some embodiments, the locking element 614 may be sized to fit on top of the base 612 such that the outer perimeter dimensions of the locking element 614 and base 612 are substantially similar. In some embodiments, the locking element 614 has a proximal or top surface 650, a distal or bottom surface 652, and a central opening 654 extending through the locking element 614 from the top surface 650 to the bottom surface 652 along the central axis. In some embodiments, the central opening 654 is sized and configured to receive a portion of the lock screw 616 therein to enable secure coupling of the locking element 614 and the lock screw 616 and to enable the lock screw 616 to exert a compressive force on the locking element 614. In some embodiments, the locking element 614 may have a perimeter recess (not shown, but identical to perimeter recess 58 of locking element 14 described above) configured to engage with the perimeter ridge 626 of the base 612 when the locking element 614 is advanced onto the base 612. In some embodiments, one or more tensionable fixation members 18 may be captured within the locking interface between the perimeter ridge 626 and the perimeter recess thereby providing the secondary locking feature as described above.

In some embodiments, the plate coupler 619 includes a head 621, neck 623, threaded post 625, and a driver recess 627 positioned at a proximal end of the threaded post 625. In some embodiments, the plate coupler 619 is configured to extend through a through-hole 502 in a fixation plate 500 such that the head 621 is on the distal (bottom or bone engaging) side of the fixation plate 500, the neck 623 is positioned within the through-hole 502, and the threaded post 625 extends proximally from the fixation plate 500. In some embodiments, the holder 617 is generally circular in shape and is sized and configured to nest within the central opening 628 of the base 612. In some embodiments, the holder 617 includes a threaded central lumen 629 configured to threadedly receive the threaded post 625 of the plate coupler 619 therein. In some embodiments, the plate coupler 619 may be secured to the fixation plate 500 by inserting the threaded post 625 through a through-hole 502 and thereafter threadedly coupling the holder 617 to the plate coupler 619 by inserting the threaded post 625 into the central lumen 629 and rotating the holder 617 to engage the threaded interaction.

In some embodiments, after securing the plate coupler 619 to the fixation plate 500, the base 612 may be placed onto the holder 617 such that the holder 617 is nested within the central opening 628. In some embodiments, the user may opt to secure the placement on the fixation plate 500 by inserting one or more purchase members (e.g., screws, wires, etc.) through one or more vertical openings 642 to ensure the base 612 remains in place during use. In some embodiments, the system 610 of the present disclosure may then be used in soft tissue-to-bone and/or bone-to-bone repair as described herein. At this point, the user may begin the repair by attaching a fixation end 18b of at least one tensionable fixation member 18 to the damaged soft tissue or bone (e.g., by passing the fixation end 18b through the tissue, bone, or other member). The free end 18a of the attached tensionable fixation member 18 may be associated with a chaperone member (e.g., needle, passing loop, etc.) and then passed through a lateral opening 638 of the base 612, for example a lateral opening 638 that is closest to and/or oriented toward the damaged tissue/bone to ensure the most efficient path to reduction. The free end 18a is then passed through the fixation channel 640 and cleat 632 associated with the selected lateral opening 638 such that the free end 18a extends proximally from the top surface 620 of the base 612. The user may then adjust the tension in the tensionable fixation member 18 to a desired level, at which point the user may then urge the tensionable fixation member 18 into the tapered extension 636 of the cleat 632 while maintaining the desired tension in the tensionable fixation member 18 to provisionally lock the tensionable fixation member 18 to the base 612 by wedging or pinching the tensionable fixation member 18 within the cleat 632.

This process may be repeated multiple times with multiple tensionable fixation members 18 passing through distinct lateral opening 638/cleat 632 combinations. In some embodiments, the fixation ends 18b of the additional tensionable fixation members 18 may be attached to the same damaged tissue and/or bone and/or prosthesis or distinct damaged tissue and/or bone and/or prosthesis. Once all the tensionable fixation members 18 have been provisionally locked within their respective cleats 632, the user may then apply the secondary lock by coupling the locking element 614 to the base 612 and securing said coupling with the lock screw 616.

By way of example, the lock screw 616 may include a head 660, a tapered distal protrusion 662 extending distally from the head 660, a textured outer circumferential surface 664, and a threaded central lumen 666 extending through the lock screw 616 along the central axis. In some embodiments, the threaded central lumen 666 is configured to engage the threaded post 625 of the plate coupler 619. In some embodiments, after placement of the locking element 614 on the base 612, the threaded post 625 of the plate coupler 619 is protruding proximally from the central opening 654 of the locking element 614. To secure the locking element 614 to the base 612, first the lock screw 616 is threaded onto the threaded post 625 of the plate coupler 619. In some embodiments, actuation of the lock screw 616 (e.g., by hand using the textured outer circumferential surface 664) advances the tapered distal protrusion 662 of the lock screw 616 into the central opening 654 of the locking element 614, which exerts a compressive force on the locking element 614, which in turn exerts a compressive force on the tensionable fixation members 18 within the locking interface described above. Final tightening may be achieved by engaging a driver tool (not shown) with the driver recess 627 of the plate coupler 619 and rotating, which will draw the lock screw 616 further toward the base 612 and exert further compression on the locking interface. At this point the construct is secured and excess free ends 18a of the tensionable fixation members 18 may be removed or optionally tied in one or more knots.

If the user determines at any point that any of the tensionable fixation members 18 need to be re-tensioned, the lock screw 616 may be rotated in the opposite direction (e.g., counterclockwise) which will release the locking element 614 from the tensionable fixation members 18, enabling the locking element 614 to be temporarily removed. Once this happens, the tensionable fixation members 18 should still be provisionally locked under the original applied tension by virtue of being wedged within their respective cleats 632. To adjust tension in any one tensionable fixation member 18, the user merely locates the tensionable fixation member at issue, pulls the tensionable fixation member 18 toward the central opening 628 to decouple the tensionable fixation member 18 from the tapered extension 636 of the cleat 632, adjust the tension to a desired level, and re-wedge the tensionable fixation member 18 within the tapered extension 636 of the cleat 632. This can be repeated independently for any tensionable fixation member 18 that needs adjusting.

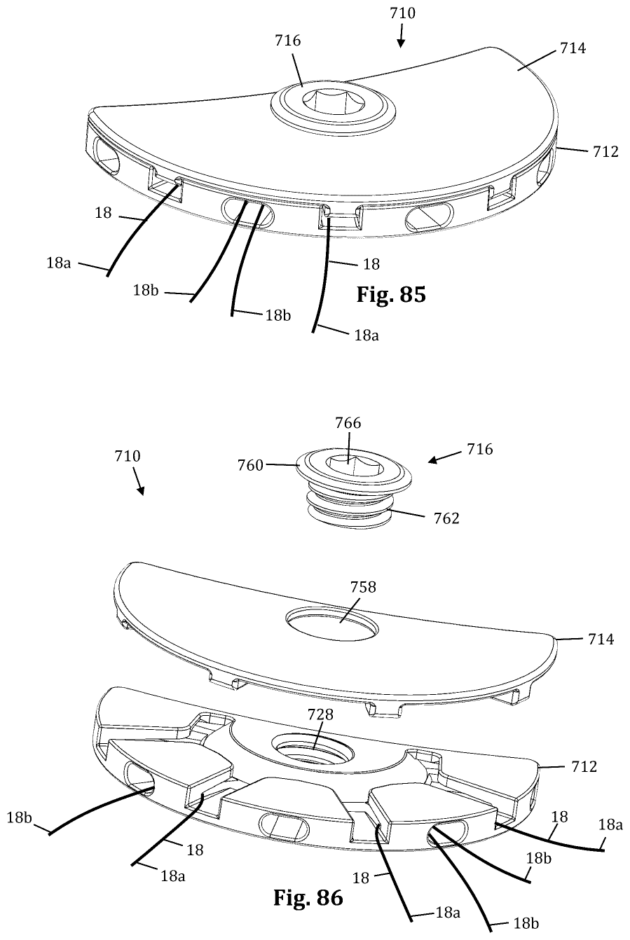

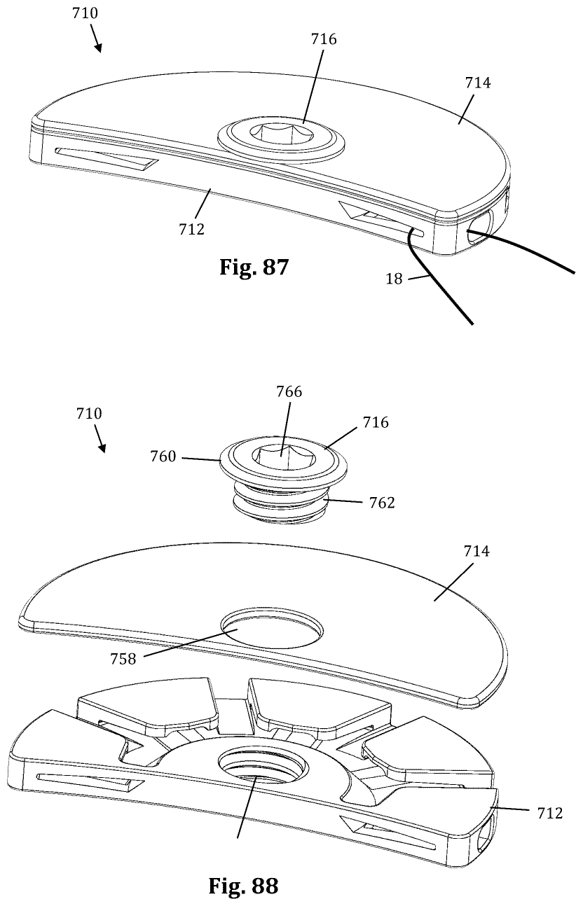

FIGS. 85-88 illustrate an example of a knotless tensionable fixation system 710 according to some embodiments of the disclosure. By way of example only, the knotless tensionable fixation system 710 (also referred to herein as “system 710”) includes a base 712, a locking element 714, a lock screw 716 and at least one tensionable fixation member 18. The system 710 of the present disclosure enables a user to achieve complex soft tissue-to-bone or bone-to-bone repair by first placing the base 712 against a first bone segment (or securing to a fixation plate or prosthesis) and then securing multiple tensionable fixation members 18 (e.g., surgical sutures, tape, wires, etc.) that may be attached to the same or different tissue or bone to be repaired (e.g., several unique tensionable fixation member strands may be attached the same tissue, or unique tensionable fixation member strands may be attached to different bone fragments or tissue segments), or to another artificial fixation member such as a prosthesis. In some embodiments, each tensionable fixation member 18 has one or more free ends 18a and a target engagement portion 18b, the target engagement portion 18b may be attached to a tissue or bone segment to be repaired or a surgical implant or prosthesis, and the tensionable fixation member 18 is passed through the base 712 such that the one or more free ends 18a extend proximally from the base 712 to enable manipulation by a user. In some embodiments, the target engagement portion 18b comprises one end of the tensionable fixation member, which then has one free end 18a. In some embodiments, the target engagement portion 18b may be a loop or middle portion of the tensionable fixation member 18, which then has two free ends 18a that may be secured by the base 712. In some embodiments, the system 710 enables each tensionable fixation member 18 to be provisionally secured to the base 712 under adequate tension while other tensionable fixation members 18 (or another strand of the same tensionable fixation member 18) are being utilized. In some embodiments, once all tensionable fixation members 18 have been provisionally secured to the base 712 under the desired tension to achieve reduction of the repair, the locking element 714 may be coupled to the base 712 and secured by the lock screw 716 under compression to provide a secondary or final lock (e.g., the provisional lock being a first lock) simultaneously to all tensionable fixation members 18 to securely lock all tensionable fixation members 18 under tension at the same time and with one single locking element 714.

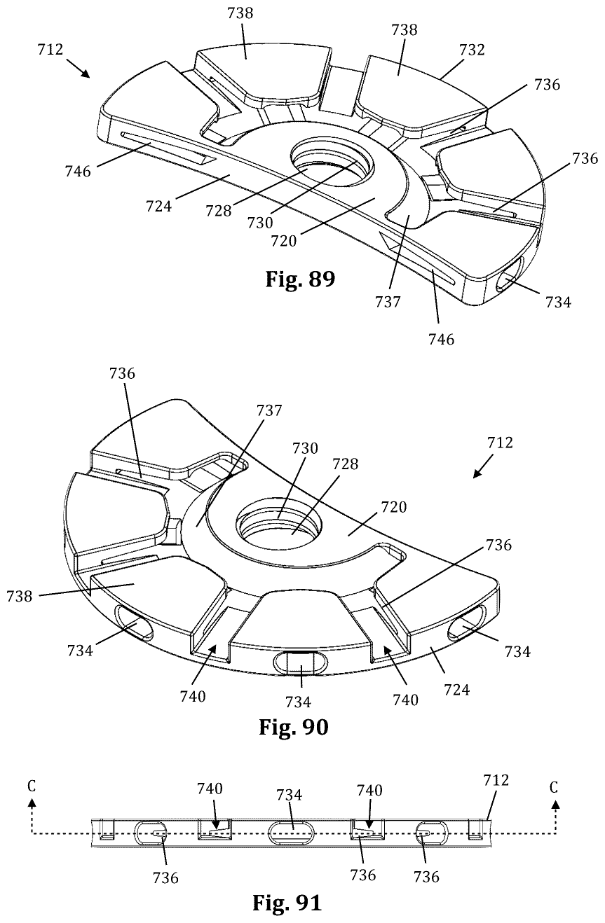

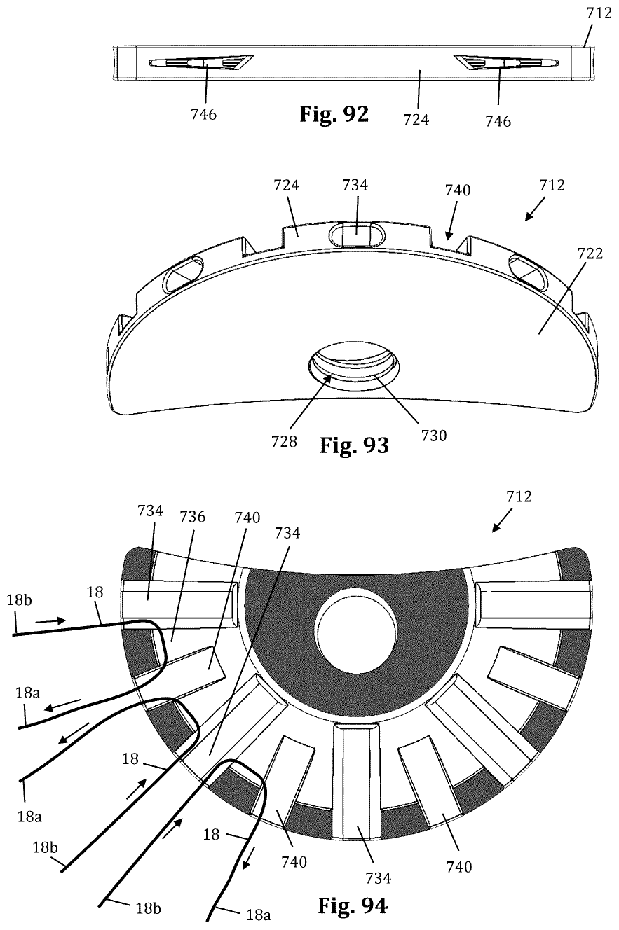

FIGS. 89-94 illustrate an example of a base 712 forming part of the system 710, according to some embodiments. By way of example, the base 712 may have any perimeter shape suitable to allow fixation of multiple tensionable fixation members 18 in multiple directions, including but not limited to circular, semi-circular, curved, half-moon (shown by way of example in FIGS. 89-94), triangular, square, oval, hexagonal, octagonal, pentagonal, and the like. In some embodiments, the base 712 may have a top or proximal surface 720, a bottom or distal surface 722, and a lateral surface 724, which may be a planar surface or a curved circumferential surface as shown, depending on the perimeter shape of the base. In some embodiments, the base 712 may have an inner cavity 726 configured to enable passage of one or more tensionable fixation members 18 therethrough.

In some embodiments, the base 712 includes central opening 728 extending through the base 712 from the top surface 720 to the bottom surface 722 along a central axis. In some embodiments, the central opening 728 includes a threaded lumen 730 configured to threadedly receive the threaded post 762 of the lock screw 716 to secure the lock screw 716 to the base 712. In some embodiments, the base 712 further comprises a plurality of provisional locking cleats 732 (hereinafter “cleats 732”) distributed around the central opening 728. In some embodiments, the plurality of cleats 732 may be positioned equidistant from one another in an annular fashion, as shown by way of example in FIG. 94, however other configurations are possible. For example, in some embodiments, the plurality of cleats 732 may be grouped or bunched to one or more sides of the base 712. In some embodiments, each cleat 732 comprises an ingress opening 734 formed in the lateral surface 724 and a pair of tapered wedge recesses 736 positioned on either side of the ingress opening 734 and extending radial inward toward the central opening 728 such that the wide end of the wedge recess 736 is nearest the central opening 728. In some embodiments, each cleat 732 further includes a guide flange 738 extending radially inward from the perimeter edge of the base 712 and configured to ensure the tensionable fixation members 18 remain within the wedge recesses 736 during use. In some embodiments, the base 712 may include an annular sloped surface 737 surrounding the central opening 728 and configured to deflect a chaperone element (such as a needle, passing loop, or guide wire used to usher the tensionable fixation member 18 through the cleat 732) away from the central opening 728 to make it easier for a user to maneuver during use.

In some embodiments, the base 712 includes a plurality of fixation channels 740 positioned between each cleat 732 and extending radially inward (toward the central opening 728). By way of example, each fixation channel 740 comprises a compression surface 744 which may be planar or curved and is configured to interact with a distal protrusion 758 of the locking element 714 to form a locking interface that captures the tensionable fixation member 18 in the secondary lock feature of the system 710. In some embodiments, each fixation channel 740 may be associated with a pair of cleats 732 such that a single distal protrusion 758 may lock one or two tensionable fixation members 18 within a single fixation channel 740 (e.g., a tensionable fixation member 18 engaged with a cleat 732 located on either side of the fixation channel 740). Thus, the base 712 is configured such that, after securing or otherwise associating a fixation end (or portion) 18b of a tensionable fixation member 18 to a tissue or bone segment to be repaired (or prosthesis), the free end 18a (or a first free end 18a) of the tensionable fixation member 18 may be passed (e.g., using a chaperone element such as a needle, passing loop, or guide wire, etc.) into the base 712 through the ingress opening 734, then guided laterally through one of the associated tapered wedge recesses 736 of the cleat 732, and then into the fixation channel 740 such that the free end 18b extends away from the fixation channel 740, as shown by way of example in FIG. 94. After pulling on the free end 18a to apply a desired amount of tension to the tensionable fixation member 18, the user may then pull radially on the tensionable fixation member 18 to urge the tensionable fixation member into (or farther into) the tapered wedge recess 736 of the cleat 732 so that the tensionable fixation member 18 is wedged or otherwise captured by the tapered wedge recess 736. This provides the primary or provisional locking of the tensionable fixation member 18 to the base 712 referred to above.

In some embodiments, the lateral surface 724 may include one or more lateral openings 746 configured to for passage of a tensionable fixation member 18 strand therethrough. In some embodiments, the one or more lateral openings may have a wedge shape to enable provisional locking of the tensionable fixation member 18 therein.

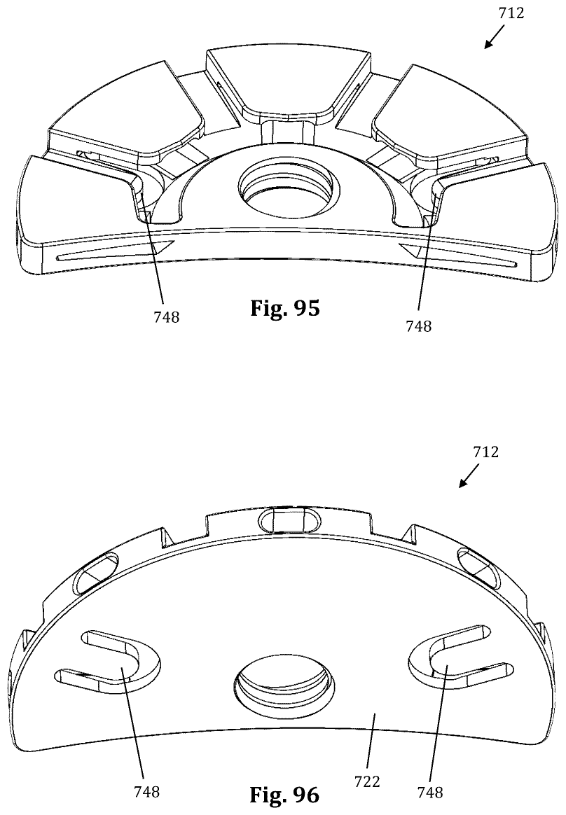

In some embodiments, the bottom surface 722 may be planar. In some embodiments, the bottom surface 722 may be contoured to more effectively rest against a bone segment or a prosthesis. In some embodiments, the bottom surface 722 may have a concave curvature. In some embodiments, the bottom surface 722 may include on or more utility flanges 748 formed therein, as shown by way of example in FIGS. 95-96. By way of example, the utility flanges 748 are configured to engage with a tensionable fixation member 18, for example a user may loop a tensionable fixation member 18 around a utility flange 748 to change the direction of the tensionable fixation member 18 within the inner cavity 726 of the base 712.

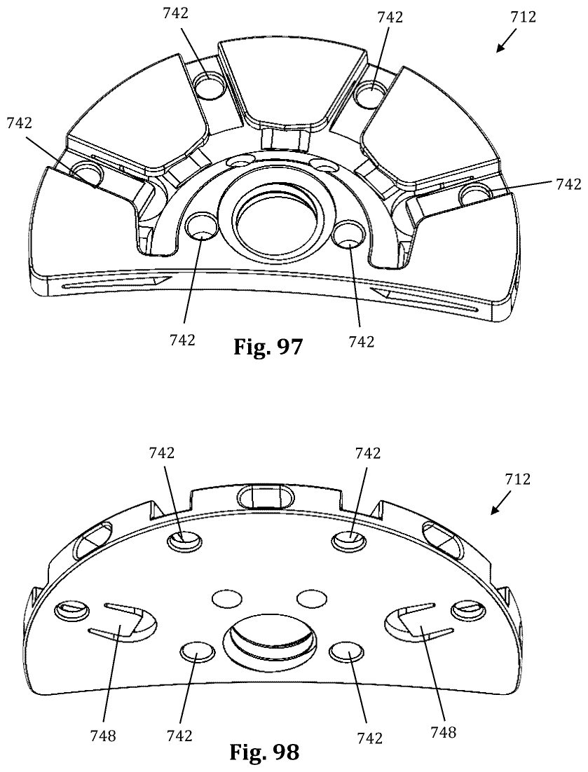

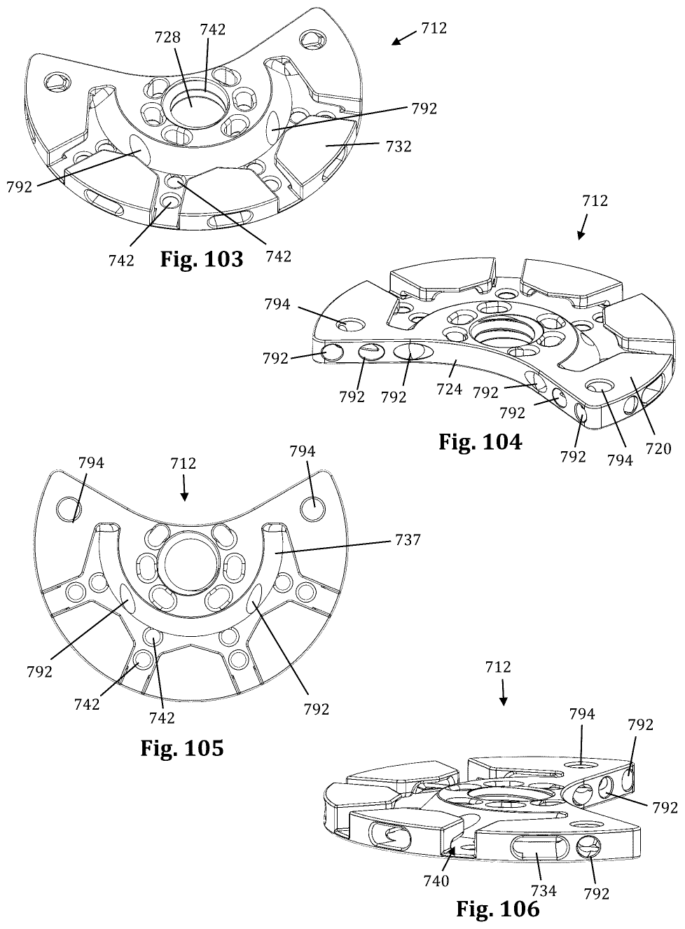

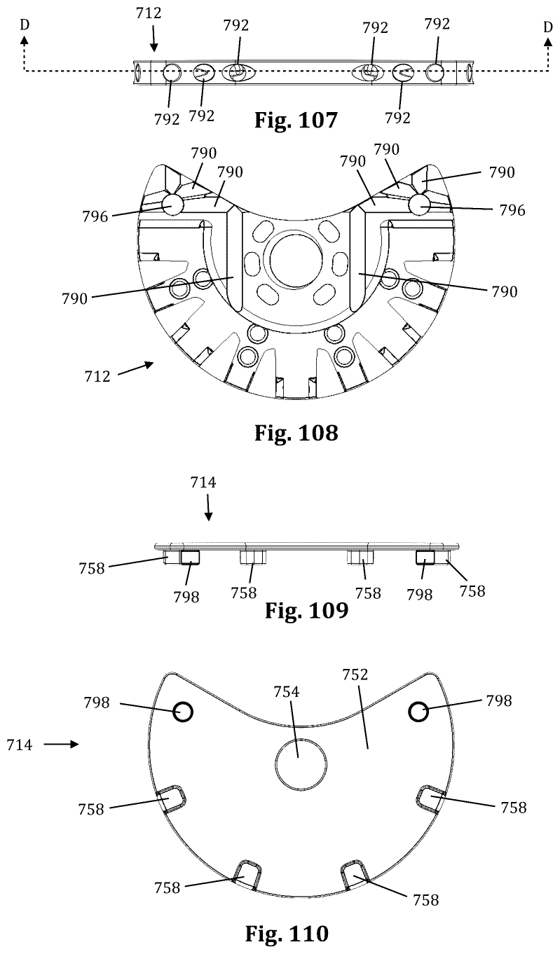

In some embodiments, the base 712 further includes one or more vertical openings 742 extending through the base 712 from the top surface 720 to the bottom surface 722, as shown by way of example in FIGS. 97-98. By way of example, the vertical openings 742 may be configured to enable passage of a purchase element (not shown) configured to extend through the vertical opening 742 and into a bone segment (or fixation plate or prosthesis) that the base 712 is positioned against in order to secure the base 712 to the bone prior to performing the repair procedure (or after commencement of the repair procedure if determined to be necessary). In some embodiments, the purchase element may be (by way of example only) a bone screw, pin, wire, tack, or any member capable of securing the base 712 to a bone. In some embodiments, the base 712 includes a plurality of vertical openings 742. In some embodiments, the plurality of vertical openings 742 may be interspersed between the cleats 732. For example, in such a case, the vertical openings 742 positioned between the cleats 732 (and within a fixation channel 740) may serve as an anchor point for a fixation portion 18b of a tensionable fixation member 18, which is then threaded through or wrapped around an object (e.g., muscle, tendon, bone, prosthesis, or other member) and returned to the base 712 such that the free ends 18a are secured to the base 712 using the cleats 732 and locking element 714. In some embodiments, a single vertical opening 742 is positioned with each fixation channel 740 as shown in FIGS. 97-98. In some embodiments, multiple vertical openings 742 may be positioned within each fixation channel 740, as shown by way of example in FIGS. 103-107. In some embodiments, the number of vertical openings 742 may be less than the number of cleats 732. In some embodiments, the vertical openings 742 may be used to pass additional tensionable fixation members 18 therethrough.

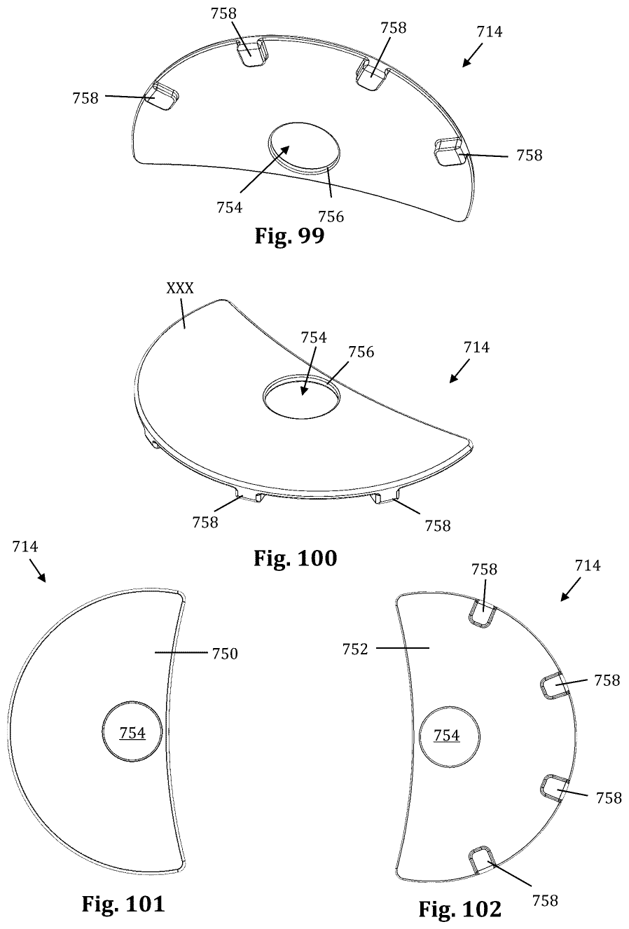

FIGS. 99-102 illustrate an example of a locking element 714, according to some embodiments. In some embodiments, the locking element 714 has an outer perimeter shape corresponding to the perimeter shape of the base 712. In some embodiments, the locking element 714 may be sized to fit on top of the base 712 such that the outer perimeter dimensions of the locking element 714 and base 712 are substantially similar. In some embodiments, the locking element 714 has a proximal or top surface 750, a distal or bottom surface 752, and a central opening 754 extending through the locking element 714 from the top surface 750 to the bottom surface 752 along the central axis. In some embodiments, the central opening 754 includes a threaded lumen 756 configured to threadedly receive the threaded post 762 of the lock screw 716 to enable secure coupling of the locking element 714 and the lock screw 716. In some embodiments, the locking element 714 may have a plurality of distal protrusions 758 extending distally from the bottom surface 752 and configured to nest within the fixation channels 740 of the base 712 when the locking element 714 is advanced onto the base 712. By way of example, the plurality of distal protrusions 758 may be distributed in a radial manner adjacent the outer perimeter edge of the locking element 714, in spatial alignment with the fixation channels 740 of the base 712 when the locking element 714 is applied to the base 712. In some embodiments, one or more tensionable fixation members 18 may be captured between the distal protrusions 758 and compression surface 744 of the fixation channel 740 (which collectively form locking interfaces) thereby providing the secondary locking feature as described above. In some embodiments, the distal protrusions 758 may have variable height dimensions to accommodate different types of tensionable fixation members 18, including but not limited to (and by way of example only) surgical sutures, tapes, wires, etc. In some embodiments, the distal protrusions 758 have a width dimension substantially similar to (but slightly smaller than) the width dimension of the fixation channels 740, so that the distal protrusions 758 may be snugly received within the fixation channels 740 to ensure alignment during association of the locking element 714 with the base 712 and to act as a counter torque feature during actuation of the lock screw 716 during final tightening, reducing or eliminating rotational friction that might otherwise cause rotational misalignment of the locking element 714 relative to the base 712.

With reference to FIGS. 86-88, the lock screw 716 may include a head 760, a threaded post 762 extending distally from the head 760, and a driver recess 766 extending into the head 760. In some embodiments, the locking element 714 is coupled to the base 712 by threadedly advancing the lock screw 716 through the central opening 754 of the locking element 714 and into the central opening 728 of the base 712. In some embodiments, the driver recess 766 is sized and shaped to facilitate engagement with a driver tool (not shown). By way of example, the threaded post 762 is configured to threadedly engage the threaded surface 730 of the central recess 728 of the base 712. In some embodiments, actuation of the lock screw 716 (e.g., by a driver tool) advances the lock screw 716 into the central opening 728 of the base 712, which causes the head 760 of the lock screw 716 to exert a compressive force on the locking element 714, which in turn exerts a compressive force on the tensionable fixation members 18 within the locking interface described above.

Referring now to FIGS. 103-110, in some embodiments, the base 712 may further include one or more transverse channels 790, each transverse channel 790 having a pair of associated channel openings 792. In some embodiments, at least one channel opening 792 is positioned on the lateral surface 724. In some embodiments, at least one channel opening is positioned on the annular sloped surface 737. In some embodiments, the base 712 includes at least one compression surface 796 positioned such that at least one transverse channel 790 passes through the compression surface 796, as shown by way of example in FIG. 108. In some embodiments, the compression surface 796 may be positioned such that more than one transverse channel 790 pass through the compression surface 796. In some embodiments, the at least one compression surface 796 is spatially aligned with at least one compression aperture 794 formed in the top surface 720 of the base 712. In some embodiments, the locking element 714 includes one or more compression posts 798 extending distally from the bottom surface 752 and positioned in alignment with the at least one compression aperture 794. In some embodiments, the at least one compression aperture 794 is sized and configured to enable passage of the compression post 798 therethrough so that the compression post 798 may contact a tensionable fixation member 18 present in the corresponding transverse channel 790 during the final tightening step to secure the tensionable fixation member 18 under compression. By way of example, in use a user may choose to guide a tensionable fixation member 18 such that it passes through one or more transverse channels, around a fracture and/or muscle and/or tendon and/or prosthesis and back to the base 712 to be secured in a cleat 732, for example.

In some embodiments, the system 710 of the present disclosure may be used in soft tissue-to-bone and/or bone-to-bone repair. To use the system 710, a user may first place the base 712 on a portion of a bone to which the damaged soft tissue and/or bone is to be attached (e.g., or the base 712 may be secured to a fixation plate or prosthesis as described herein). The user may then opt to secure the placement on the bone by inserting one or more purchase members (e.g., screws, wires, etc.) through one or more vertical openings 742 to ensure the base 712 remains in place during use. At this point, the user may begin the repair by attaching a fixation end 18b of at least one tensionable fixation member 18 to the damaged soft tissue or bone (e.g., by passing the fixation end 18b through the tissue, bone, or other member). The free end 18a of the attached tensionable fixation member 18 may be associated with a chaperone member (e.g., needle, passing loop, etc.) and then passed through an ingress opening 734 of the base 712, for example an ingress opening 734 that is closest to and/or oriented toward the damaged tissue/bone to ensure the most efficient path to reduction. The free end 18a is then passed through one of the lateral tapered wedge recesses 736 of the cleat 732 associated with the selected ingress opening 734 such that the free end 18a extends into and through the fixation channel 740. The user may then adjust the tension in the tensionable fixation member 18 to a desired level, at which point the user may then wedge the tensionable fixation member 18 into the lateral tapered wedge recess 736 of the cleat 732 while maintaining the desired tension in the tensionable fixation member 18 to provisionally lock the tensionable fixation member 18 to the base 712 by wedging or pinching the tensionable fixation member 18 within the cleat 732.

This process may be repeated multiple times with multiple tensionable fixation members 18 passing through various ingress opening 734/cleat 732 combinations. In some embodiments, the fixation ends 18b of the additional tensionable fixation members 18 may be attached to the same damaged tissue and/or bone and/or prosthesis or distinct damaged tissue and/or bone and/or prosthesis. Once all the tensionable fixation members 18 have been provisionally locked within their respective cleats 732, the user may then apply the secondary lock by coupling the locking element 714 to the base 712. The secondary locking step may be accomplished by aligning the threaded post 762 of the lock screw 716 with the central opening 728 of the base 712, and then rotationally advancing the lock screw 716 into the central opening 728 until the locking element 714 contacts the tensionable fixation members 18. Final tightening (e.g., the secondary lock) is then accomplished by further advancement of the lock screw 716. Once the locking element 714 is in contact with the tensionable fixation members 18, further rotation of the lock screw 716 will cause the locking element 714 to compress the tensionable fixation members 18. This will effectively pinch the tensionable fixation members 18 within the locking interfaces between the compression surfaces 744 within the fixation channel 740 of the base 712 and the distal protrusions 758 of the locking element 714. At this point the construct is secured and excess free ends 18a of the tensionable fixation members 18 may be removed or optionally tied in one or more knots.

If the user determines at any point that any of the tensionable fixation members 18 need to be re-tensioned, the lock screw 716 may be rotated in the opposite direction (e.g., counterclockwise) which will release the locking element 714 from the tensionable fixation members 18, enabling the locking element 714 to be temporarily removed. Once this happens, the tensionable fixation members 18 should still be provisionally locked under the original applied tension by virtue of being wedged within their respective cleats 732. To adjust tension in any one tensionable fixation member 18, the user merely locates the tensionable fixation member at issue, pulls the tensionable fixation member 18 toward the central opening 728 to decouple the tensionable fixation member 18 from the tapered wedge recess 736 of the cleat 732, adjust the tension to a desired level, and re-wedge the tensionable fixation member 18 within the tapered wedge recess 736 of the cleat 732. This can be repeated independently for any tensionable fixation member 18 that needs adjusting.

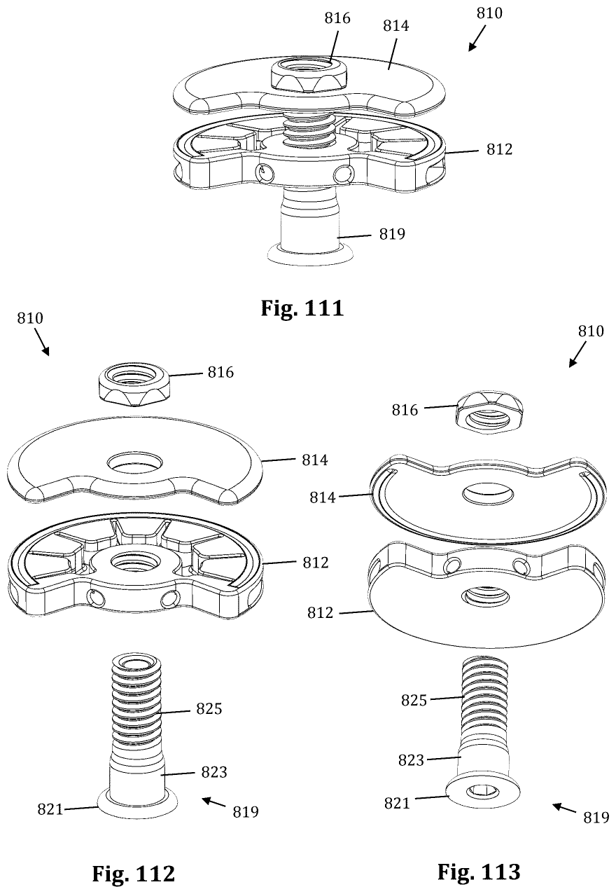

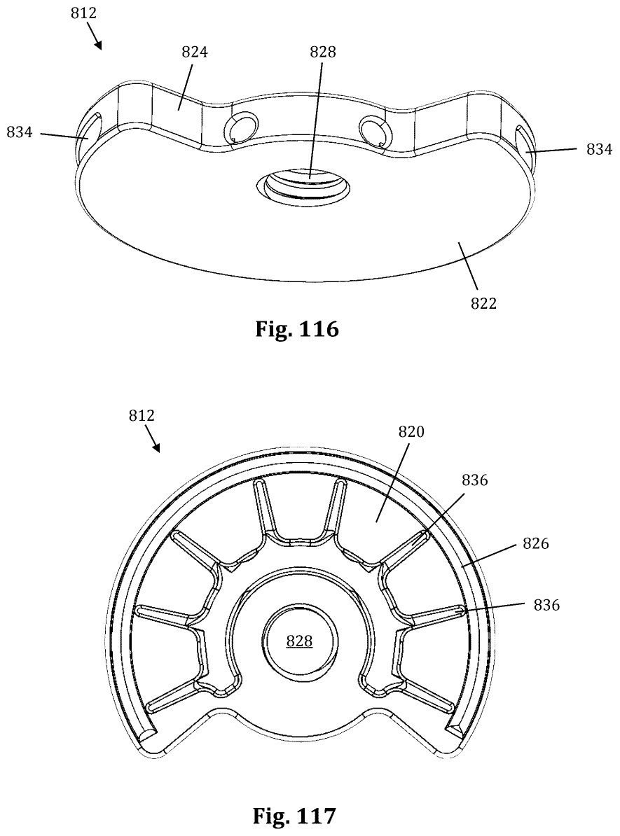

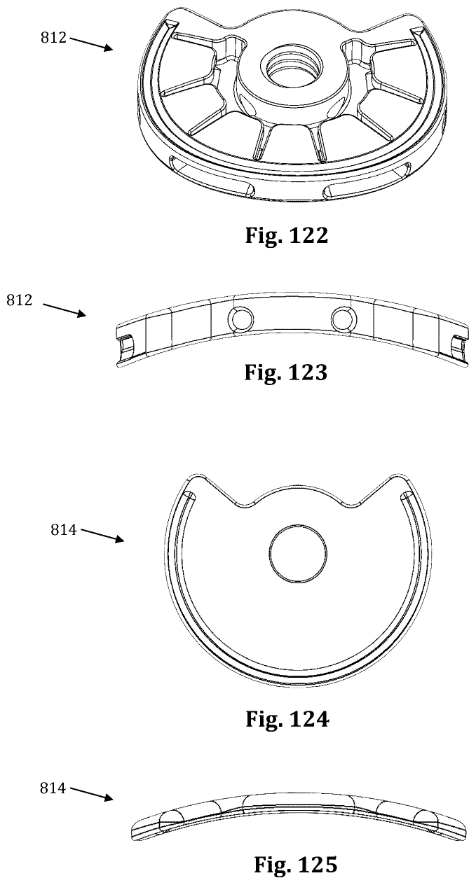

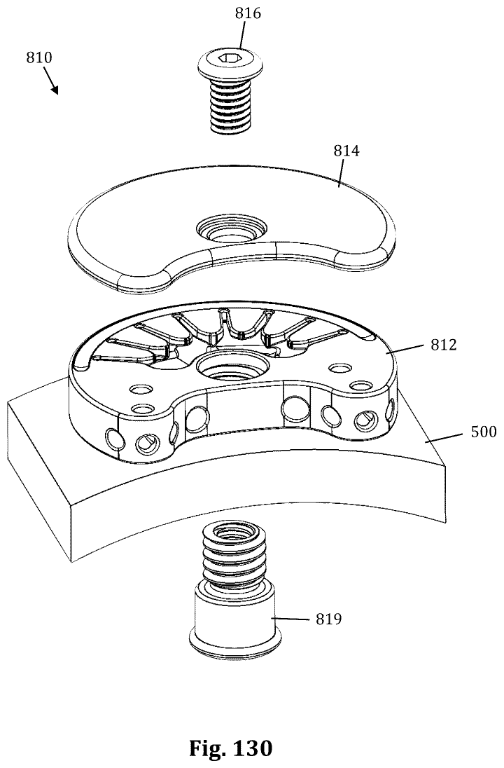

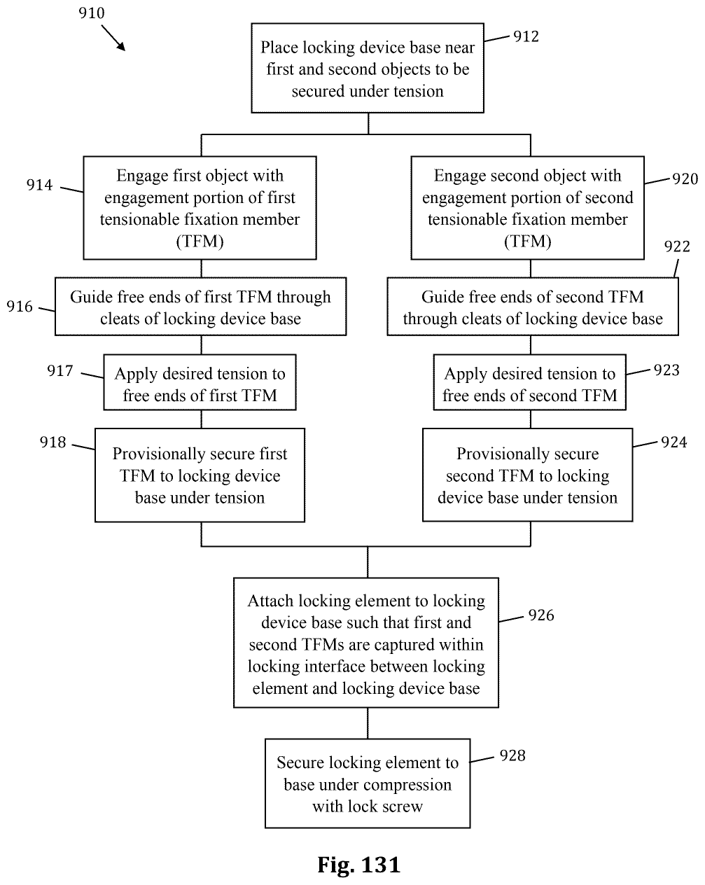

FIGS. 111-130 illustrate an example of a knotless tensionable fixation system 810 according to some embodiments of the disclosure. By way of example only, the knotless tensionable fixation system 810 (also referred to herein as “system 810”) includes a base 812, a locking element 814, a lock screw 816, and a plate coupler 819. In some embodiments, the system 810 is configured for coupling with a bone fixation plate 500. By way of example, the system 810 of the present disclosure enables a user to achieve complex soft tissue-to-bone or bone-to-bone repair by first securing the base 812 to a fixation plate 500 and then securing multiple tensionable fixation members 18 (e.g., surgical sutures, wires, tapes, etc.) that may be attached to the same or different tissue or bone to be repaired (e.g., several unique tensionable fixation member strands may be attached the same tissue, or unique tensionable fixation member strands may be attached to different bone fragments or tissue segments), or to another artificial fixation member such as a prosthesis. In some embodiments, each tensionable fixation member 18 has one or more free ends 18a and a target engagement portion 18b, the target engagement portion 18b may be attached to a tissue or bone segment to be repaired or a surgical implant or prosthesis, and the tensionable fixation member 18 is passed through the base 812 such that the one or more free ends 18a extend proximally from the base 812 to enable manipulation by a user. In some embodiments, the target engagement portion 18b comprises one end of the tensionable fixation member, which then has one free end 18a. In some embodiments, the target engagement portion 18b may be a loop or middle portion of the tensionable fixation member 18, which then has two free ends 18a that may be secured by the base 812. In some embodiments, the system 810 enables each tensionable fixation member 18 to be provisionally secured to the base 812 under adequate tension while other tensionable fixation members 18 (or another strand of the same tensionable fixation member 18) are being utilized. In some embodiments, once all tensionable fixation members 18 have been provisionally secured to the base 812 under the desired tension to achieve reduction of the repair, the locking element 814 may be coupled to the base 812 and secured by the lock screw 816 under compression to provide a secondary or final lock (e.g., the provisional lock being a first lock) simultaneously to all tensionable fixation members 18 to securely lock all tensionable fixation members 18 under tension at the same time and with one single locking element 814.

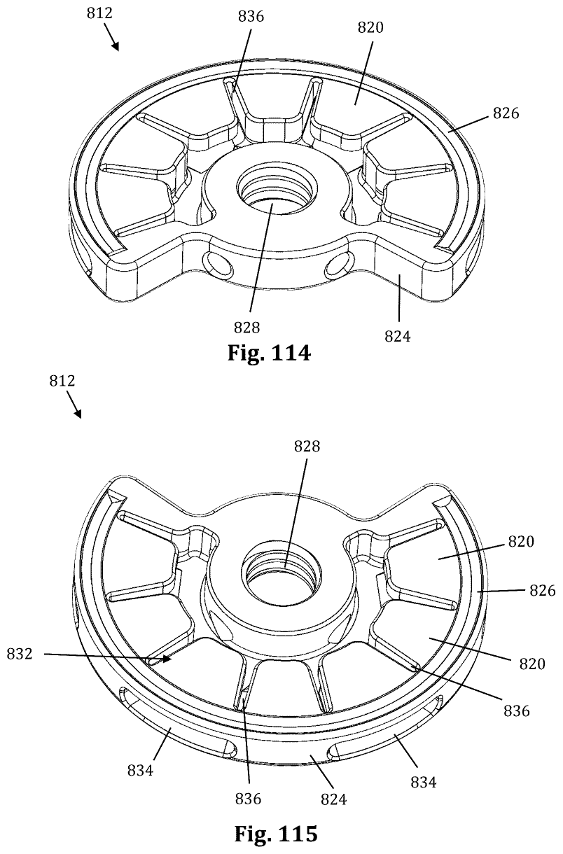

Referring to FIGS. 114-117, and by way of example only, the base 812 may have any perimeter shape suitable to allow fixation of multiple tensionable fixation members 18 in multiple directions, including but not limited to circular, semi-circular, curved, half-moon, kidney-shaped, triangular, square, oval, hexagonal, octagonal, pentagonal, and the like. In some embodiments, the base 812 may have a perimeter recess 826 configured to engage with a perimeter ridge 858 of the locking element 814 when the locking element 814 is advanced onto the base 812. In some embodiments, the perimeter recess 826 of the base 812 and perimeter ridge 858 of the locking element 814 cooperate to form a “locking interface” configured to capture and compress the tensionable fixation members 18 therebetween upon actuation of the lock screw 816, thereby providing the secondary locking feature.

In some embodiments, the base 812 includes a top surface 820, bottom surface 822, a lateral surface 824 defining the outer perimeter of the base 812, and a threaded central opening 828 extending through the base 812 and configured to receive the threaded post 914 of the plate coupler 819 therein. In some embodiments, the base 812 further comprises a plurality of provisional locking cleats 832 (hereinafter “cleats 832”) distributed around the central opening 828. In some embodiments, the plurality of cleats 832 may be positioned equidistant from one another in an annular fashion, as shown by way of example in FIG. 112, however other configurations are possible. For example, in some embodiments, the plurality of cleats 832 may be grouped or bunched to one or more sides of the base 812. In some embodiments, each cleat 832 is accessible by at least one ingress opening 834 formed in the lateral surface 824 and comprises a tapered wedge recess 836 formed in the top surface 820 positioned near an ingress opening 834 and extending radial inward toward the central opening 828 such that the wide end of the wedge recess 836 is nearest the central opening 828 and the narrow end of the wedge recess 836 is near the outer perimeter edge of the base 812.

Thus, the base 812 is configured such that, after securing a fixation end (or portion) 18b of a tensionable fixation member 18 to a tissue or bone segment to be repaired, the free end(s) 18a of the tensionable fixation member 18 may be passed (e.g., using a chaperone element such as a needle, passing loop, or guide wire, etc.) into the base 812 through the ingress opening 834 and emerging through the top surface 820 through a tapered wedge recess 836 of an adjacent cleat 832. After pulling on the free end 18a to apply a desired amount of tension to the tensionable fixation member 18, the user may then pull proximally on the tensionable fixation member 18 to urge the tensionable fixation member into (or farther into) the tapered wedge recess 836 of the cleat 832 so that the tensionable fixation member 18 is wedged or otherwise captured by the wedge recess 836. This provides the initial or provisional locking of the tensionable fixation member 18 to the base 812 referred to above.

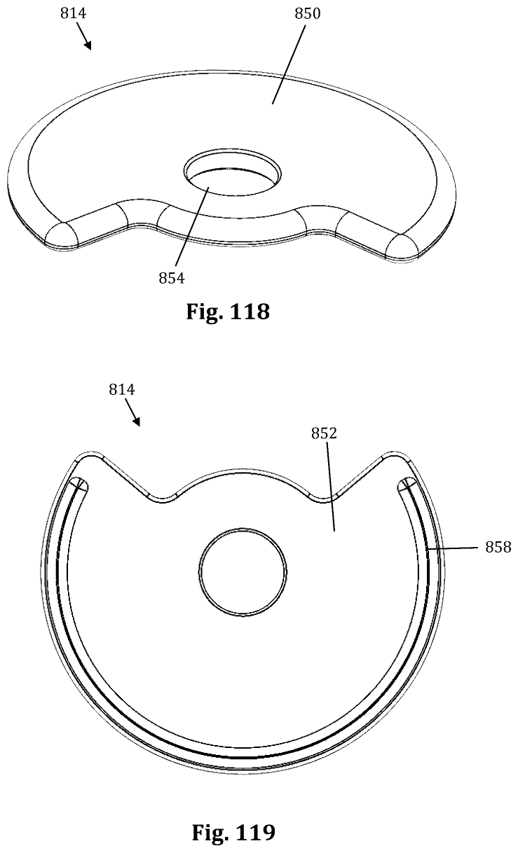

Referring now to FIGS. 118-119, in some embodiments, the locking element 814 has an outer perimeter shape corresponding to the perimeter shape of the base 812. In some embodiments, the locking element 814 may be sized to fit on top of the base 812 such that the outer perimeter dimensions of the locking element 814 and base 812 are substantially similar. In some embodiments, the locking element 814 has a proximal or top surface 850, a distal or bottom surface 852, and a central opening 854 extending through the locking element 814 from the top surface 850 to the bottom surface 852 along the central axis. In some embodiments, the central opening 854 is sized and configured to receive a portion of the plate coupler 819 therethrough to facilitate engagement of the lock screw 816 to the plate coupler 819, which enables the lock screw 816 to exert a compressive force on the locking element 814. In some embodiments, the locking element 814 may have a perimeter ridge 858 configured to engage with the perimeter recess 826 of the base 812 when the locking element 814 is advanced onto the base 812. In some embodiments, one or more tensionable fixation members 18 may be captured within the locking interface between the perimeter ridge 858 and the perimeter recess 826 thereby providing the secondary locking feature as described above.

With reference to FIGS. 111-113, in some embodiments, the plate coupler 819 includes a head 821, neck 823, and a threaded post 825. In some embodiments, the plate coupler 819 is configured to extend through a through-hole 502 in a fixation plate 500 such that the head 821 is on the distal (bottom or bone engaging) side of the fixation plate 500, the neck 823 is positioned within the through-hole 502, and the threaded post 825 extends proximally from the fixation plate 500. In some embodiments, the plate coupler 819 may be secured to the fixation plate 500 by inserting the threaded post 825 through a through-hole 502 and thereafter threadedly coupling the plate coupler 819 to the base 812 by inserting the threaded post 825 into the central opening 828 and rotating the base 812 until secured to the plate 500.

In some embodiments, the system 810 of the present disclosure may then be used in soft tissue-to-bone and/or bone-to-bone repair as described herein. At this point, the user may begin the repair by attaching a fixation end 18b of at least one tensionable fixation member 18 to the damaged soft tissue or bone (e.g., by passing the fixation end 18b through the tissue, bone, or other member). The free end 18a of the attached tensionable fixation member 18 may be associated with a chaperone member (e.g., needle, passing loop, etc.) and then passed through an ingress opening 834 of the base 812, for example an ingress opening 834 that is closest to and/or oriented toward the damaged tissue/bone to ensure the most efficient path to reduction. The free end 18a is then passed through the tapered wedge recess 836 of an adjacent cleat 632 such that the free end 18a extends proximally from the top surface 820 of the base 812. The user may then adjust the tension in the tensionable fixation member 18 to a desired level, at which point the user may then urge the tensionable fixation member 18 into the tapered wedge recess 836 of the cleat 832 while maintaining the desired tension in the tensionable fixation member 18 to provisionally lock the tensionable fixation member 18 to the base 812 by wedging or pinching the tensionable fixation member 18 within the cleat 832.

This process may be repeated multiple times with multiple tensionable fixation members 18 passing through distinct lateral opening 834/cleat 832 combinations. In some embodiments, the fixation ends 18b of the additional tensionable fixation members 18 may be attached to the same damaged tissue and/or bone and/or prosthesis or distinct damaged tissue and/or bone and/or prosthesis. Once all the tensionable fixation members 18 have been provisionally locked within their respective cleats 832, the user may then apply the secondary lock by coupling the locking element 814 to the base 812 and securing said coupling with the lock screw 816, for example by threading the lock screw 816 onto the threaded post 825 of the plate coupler 819.

With continued reference to FIGS. 111-113, the lock screw 816 may include a head 860 including a distal surface 862, a textured outer circumferential surface 864, and a threaded central lumen 866 extending through the lock screw 816 along the central axis. In some embodiments, the threaded central lumen 866 is configured to engage the threaded post 825 of the plate coupler 819. In some embodiments, after placement of the locking element 814 on the base 812, the threaded post 825 of the plate coupler 819 is protruding proximally from the central opening 854 of the locking element 814. To secure the locking element 814 to the base 812, first the lock screw 816 is threaded onto the threaded post 825 of the plate coupler 819. In some embodiments, actuation of the lock screw 816 (e.g., by hand using the textured outer circumferential surface 864) advances the distal surface 862 of the lock screw 816 into contact with the top surface 850 around the central opening 854 of the locking element 814, which exerts a compressive force on the locking element 814, which in turn exerts a compressive force on the tensionable fixation members 18 within the locking interface described above. At this point the construct is secured and excess free ends 18a of the tensionable fixation members 18 may be removed or optionally tied in one or more knots.

If the user determines at any point that any of the tensionable fixation members 18 need to be re-tensioned, the lock screw 816 may be rotated in the opposite direction (e.g., counterclockwise) which will release the locking element 814 from the tensionable fixation members 18, enabling the locking element 814 to be temporarily removed. Once this happens, the tensionable fixation members 18 should still be provisionally locked under the original applied tension by virtue of being wedged within their respective cleats 832. To adjust tension in any one tensionable fixation member 18, the user merely locates the tensionable fixation member at issue, pulls the tensionable fixation member 18 toward the central opening 828 to decouple the tensionable fixation member 18 from the tapered wedge recess 836 of the cleat 832, adjust the tension to a desired level, and re-wedge the tensionable fixation member 18 within the tapered wedge recess 836 of the cleat 832. This can be repeated independently for any tensionable fixation member 18 that needs adjusting.

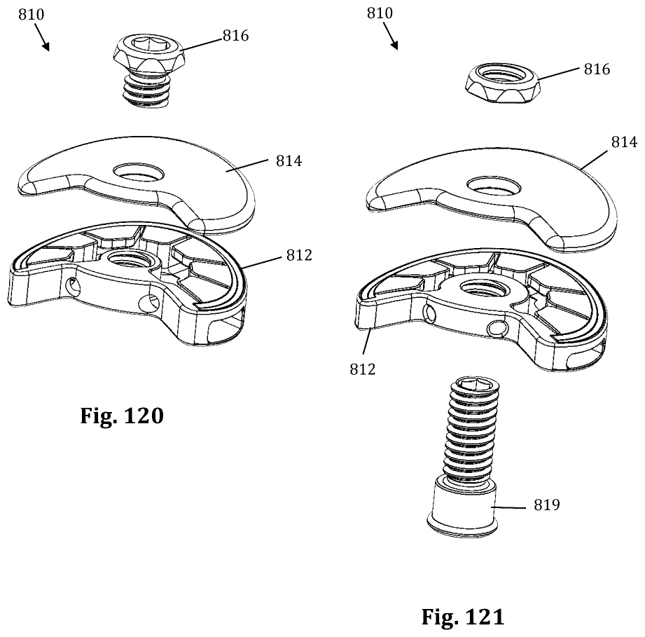

By way of example, FIGS. 120-125 illustrate examples of the system 810 wherein the base 812 and locking element 814 have an overall concave curvature. By way of example, this configuration can be advantageous to ensure a more secure interaction with a contoured anatomy, curved plate, or curved prosthesis.

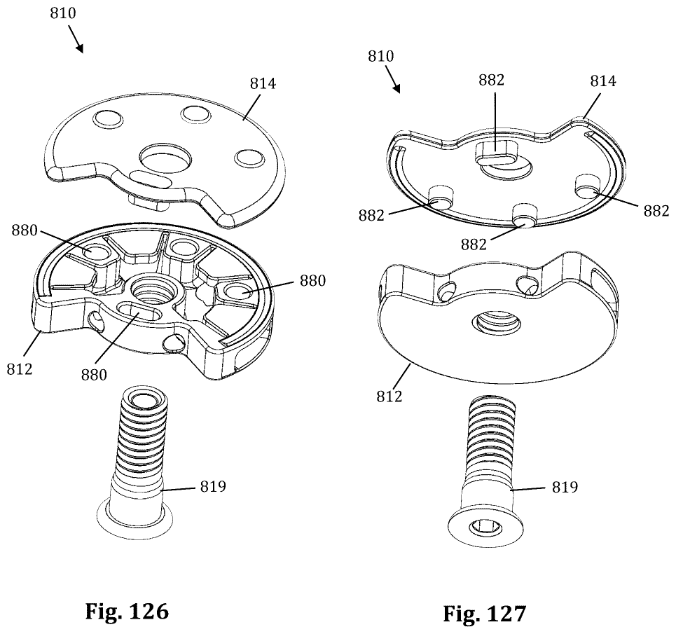

Referring to FIGS. 126-127, in some embodiments, the base plate 812 may have one or more alignment apertures 880 formed in the top surface 820, configured to receive one or more alignment posts 882 extending distally from the bottom surface 852 of the locking element 814. By way of example, the alignment posts 882, when received in the alignment apertures 880 upon placement of the locking element 814 on the base 812, prevent movement of the locking element 814 during final tightening of the construct. In some embodiments, the alignment posts 882 and alignment apertures 880 have complementary shapes to ensure a snug interaction, including but not limited to (and by way of example only), cylindrical, circular, oblong, triangular, square, rectangular, and the like.

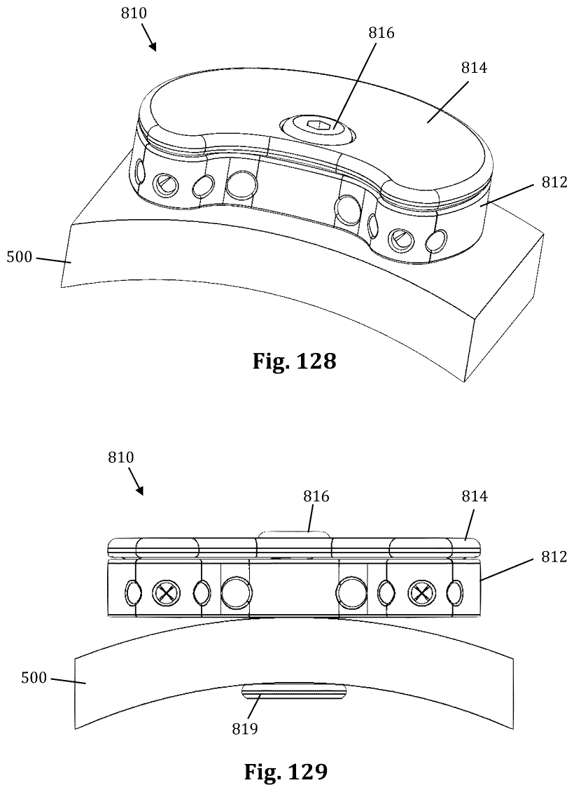

Referring to FIGS. 128-130, in some embodiments, the system 810 may be coupled to the plate 500 using a plate coupler 819 and a lock screw 816 that threads into the plate coupler 819.

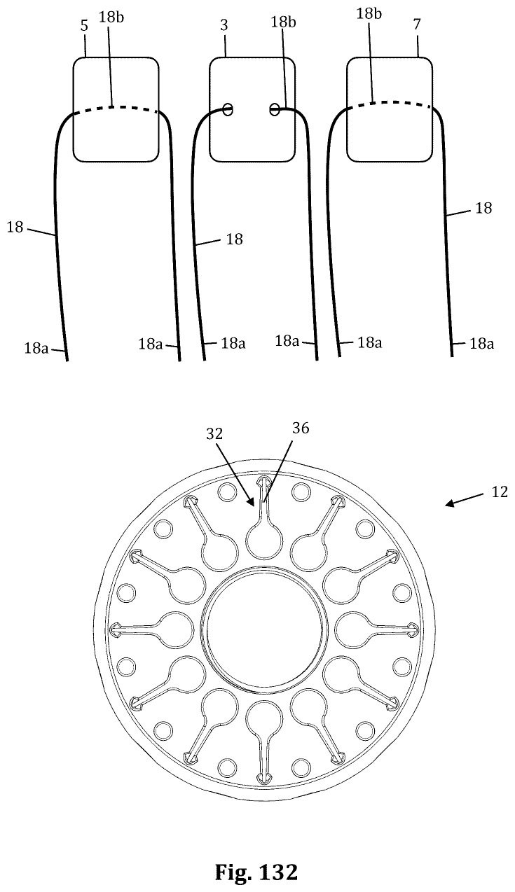

Referring to FIGS. 131-136, in some embodiments, any of the multi-strand knotless tensionable fixation systems described herein may be used in a method 910 of securing at least two objects under tension with at a plurality of tensionable fixation members. In some embodiments, the method 910 may be used in orthopedic fracture fixation procedures, in which case the objects to be secured under tension may comprise one or more of bone (e.g., fractured sections of bone, tuberosities), muscle, tendon, and/or surgical implants. By way of example the method 910 is described herein using the system 10, described above with reference to FIGS. 1-14, however this method can be following using any of the multi-strand knotless tensionable fixation systems 10, 110, 210, 410, 610, 710, 810 described herein. In some embodiments, a first step 912 in the method 910 is to place a base 12 of the multi-strand knotless tensionable fixation system 10 near first and second objects to be secured under tension as shown by way of example in FIG. 132. For example, as shown in FIG. 132, the base 12 may be placed near a first object 3, which in some embodiments may represent a fractured bone or surgical prosthesis, a second object 5, which in some embodiments, may represent a muscle, tendon or bone fragment (e.g., fractured tuberosities in a shoulder repair scenario) or surgical implant, and a third object 7, which in some embodiments, may represent a muscle, tendon bone fragment (e.g., fractured tuberosities in a shoulder repair scenario) or surgical implant.

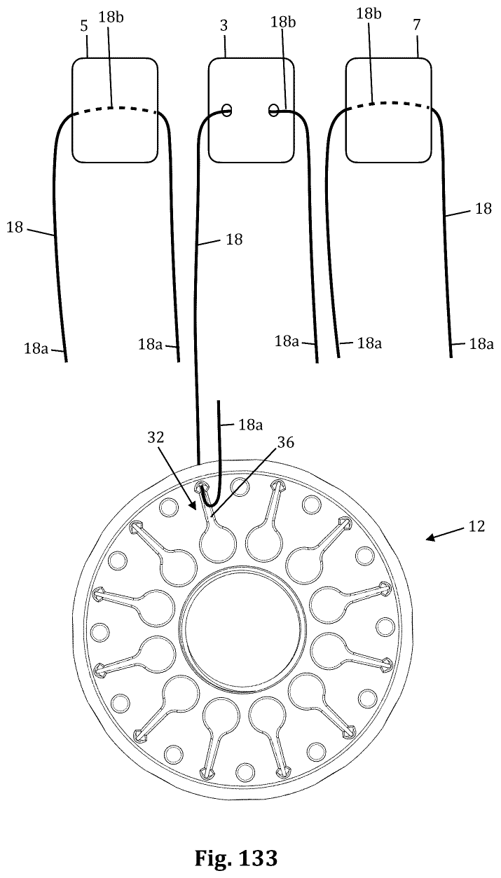

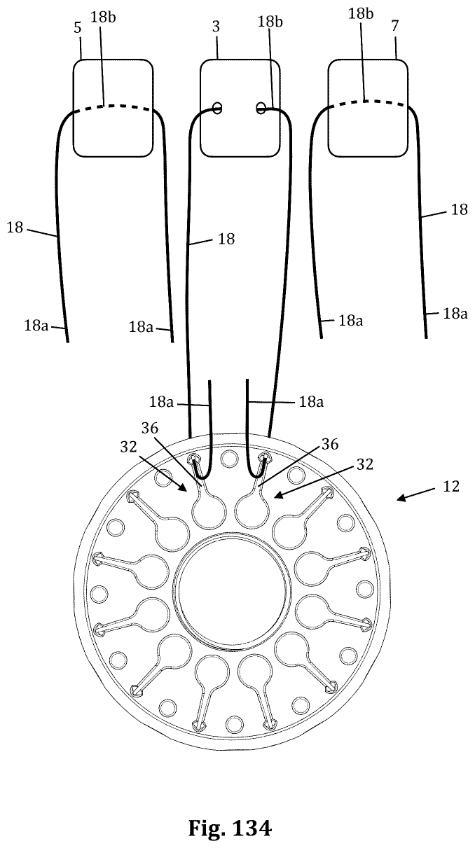

In some embodiments, a next step 914 of the method 910 is to engage the first object 3 with an engagement portion 18b of a first tensionable fixation member (TFM) 18. By way of example, this may be done by passing the first TFM 18 through the object 3 or by wrapping the first TFM 18 around the object 3. In some embodiments, a next step 916 of the method 910 is to guide a first free end 18a of the first TFM 18 through a first provisional locking cleat 32 of the base 12, as shown by way of example in FIG. 133. In some embodiments, a next step 917 of the method 910 is to apply a desired tension to first TFM 18. In some embodiments, a next step 918 of the method 910 is to provisionally secure the first free end 18a of the first TFM 18 to the base 12 under tension, for example by pulling on the first free end 18a to wedge the first free end 18a into tapered end 36 of the cleat 32, as described above. In some embodiments, these steps 914-918 may be repeated for the second free end 18a of the first TFM 18, with the end result being that the second free end 18a is wedged into a second provisional locking cleat 32 under tension, as shown by way of example in FIG. 134. Once both the first and second free end 18a of the TFM 18 have been provisionally secured within the first and second cleats 32, the user (e.g., surgeon) may proceed with securing a second TFM 18 to the base 12.

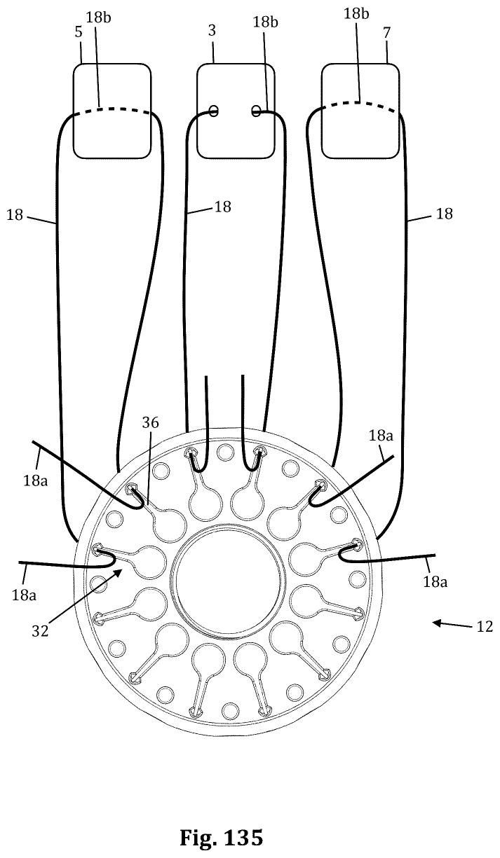

In some embodiments, a next step 920 in the method 910 is to engage the second object 5 with an engagement portion 18b of a second tensionable fixation member (TFM) 18. By way of example, this may be done by passing the second TFM 18 through the object 5 or by wrapping the second TFM 18 around the object 5. In some embodiments, a next step 922 of the method 910 is to guide a first free end 18a of the second TFM 18 through a third provisional locking cleat 32 of the base 12, as shown by way of example in FIG. 135. In some embodiments, a next step 923 of the method 910 is to apply a desired tension to second TFM 18. In some embodiments, a next step 924 of the method 910 is to provisionally secure the first free end 18a of the second TFM 18 to the base 12 under tension, for example by pulling on the first free end 18a to wedge the first free end 18a into tapered end 36 of the third cleat 32, as described above. In some embodiments, these steps 920-924 may be repeated for the second free end 18a of the second TFM 18, with the end result being that the second free end 18a is wedged into a fourth provisional locking cleat 32 under tension, as shown by way of example in FIG. 135. In some embodiments, steps 914-924 may be repeated as many times as needed to secure the desired number of TFMs 18 to the base 12.

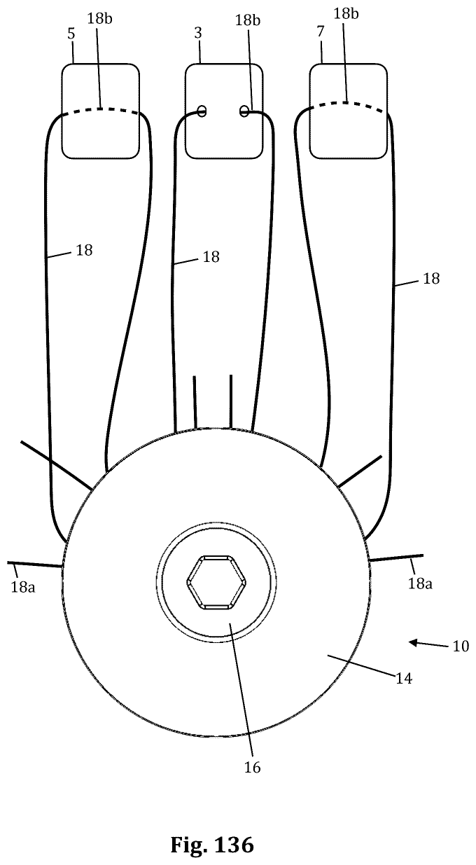

Once all desired TFMs 18 have been secured to their target objects and to the base 12, a next step 926 in the method 910 is to attach the locking element 14 to the base 12 such that the first and second (and third, fourth, etc.) TFMs 18 are captured within the locking interface between the locking element 14 and the base 12, as described above. In some embodiments, a next step 928 in the method 910 is to secure the locking element 14 to the base under compression with the lock screw 16, as shown by way of example in FIG. 136. At this point, the excess tensionable fixation member 18 material may be cut off, tied into a knot (for additional but unnecessary security) or simply left alone.

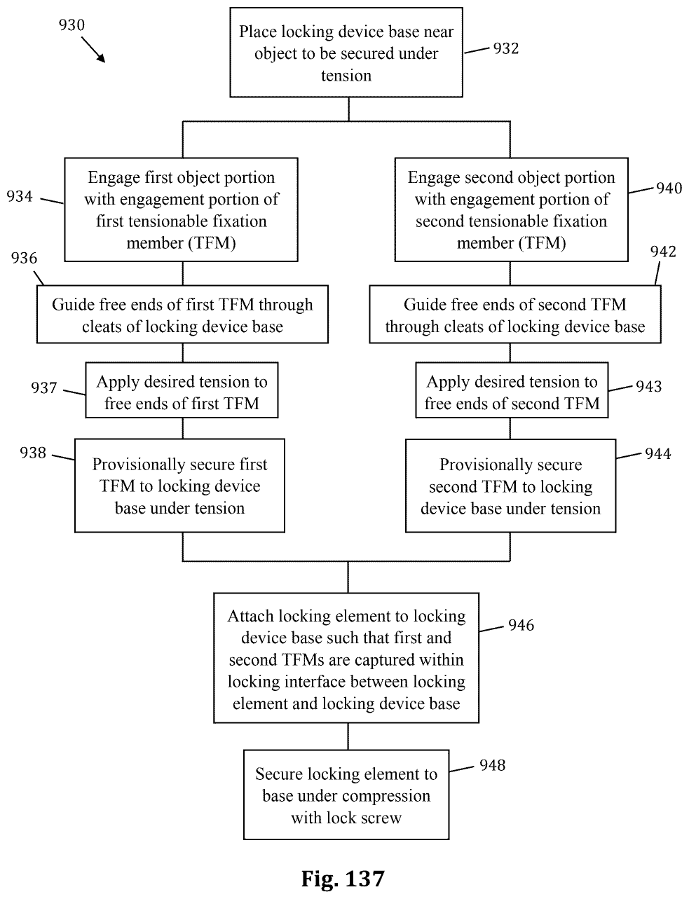

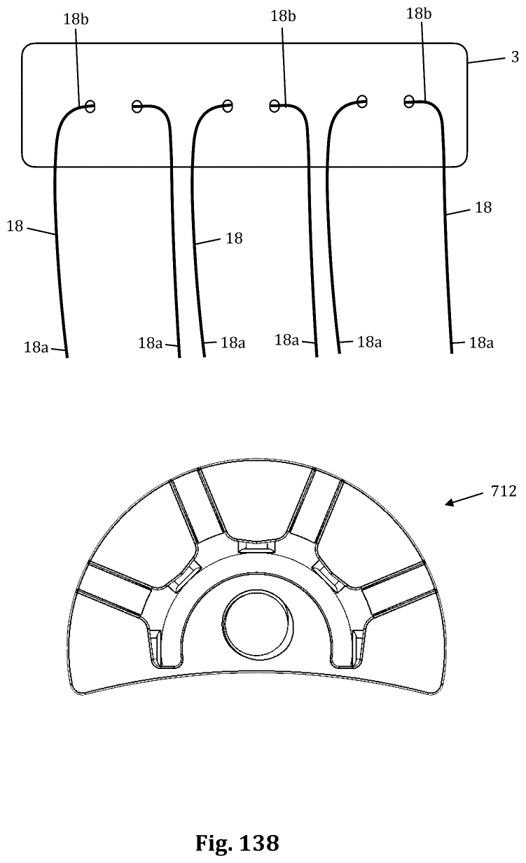

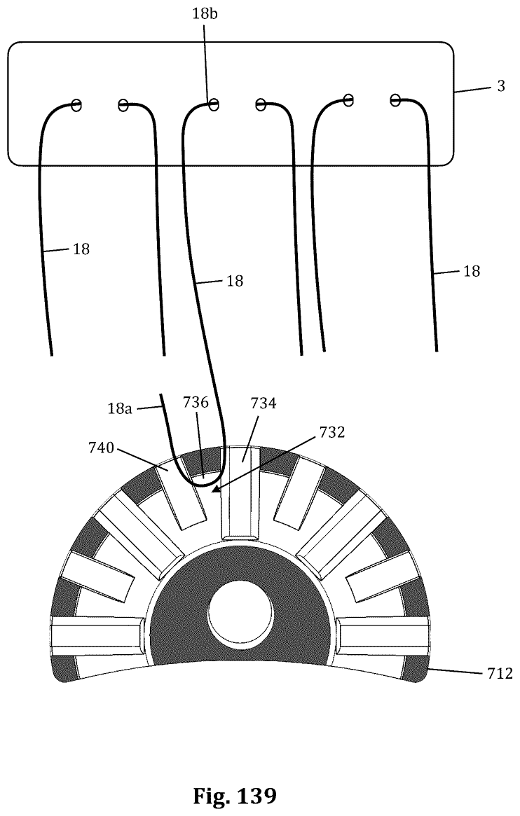

Referring to FIGS. 137-142, in some embodiments, any of the multi-strand knotless tensionable fixation systems described herein may be used in a method 930 of securing multiple portions of a singular object under tension with at a plurality of tensionable fixation members. In some embodiments, a first step 932 in the method 930 is to Referring to FIGS. 137-142, in some embodiments, any of the multi-strand knotless tensionable fixation systems described herein may be used in a method 930 of securing at least two portions of a singular object 3 under tension with at a plurality of tensionable fixation members 18. In some embodiments, the method 930 may be used in orthopedic fracture fixation procedures, in which case the objects to be secured under tension may comprise one or more of bone (e.g., fractured sections of bone), muscle, tendon, and/or surgical implants. By way of example the method 930 is described herein using the system 710, described above with reference to FIGS. 85-110, however this method can be following using any of the multi-strand knotless tensionable fixation systems 10, 110, 210, 410, 610, 710, 810 described herein. In some embodiments, a first step 932 in the method 930 is to place a base 712 of the multi-strand knotless tensionable fixation system 710 an object to be secured under tension as shown by way of example in FIG. 138. For clarity, FIGS. 139-141 shown a cross-section of the base 712 to ensure that the path followed by the tensionable fixation members 18 is clearly shown.

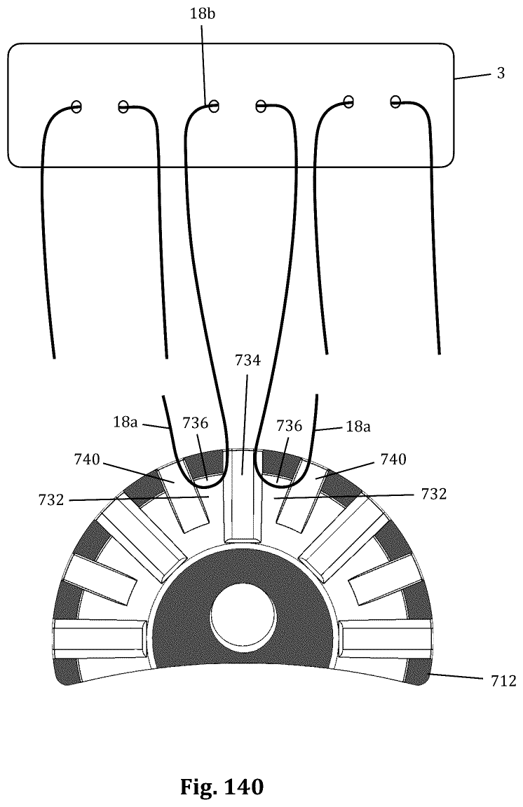

In some embodiments, a next step 934 of the method 930 is to engage a first portion of the object 3 with an engagement portion 18b of a first tensionable fixation member (TFM) 18. By way of example, this may be done by passing the first TFM 18 through the object 3 or by wrapping the first TFM 18 around the object 3. In some embodiments, a next step 936 of the method 930 is to guide a first free end 18a of the first TFM 18 through a first provisional locking cleat 732 of the base 712, as shown by way of example in FIG. 139. In some embodiments, a next step 937 of the method 930 is to apply a desired tension to the first TFM 18. In some embodiments, a next step 938 of the method 930 is to provisionally secure the first free end 18a of the first TFM 18 to the base 712 under tension, for example by pulling on the first free end 18a to wedge the first free end 18a into the lateral tapered wedge recess 736 of the cleat 732, as described above. In some embodiments, these steps 934-938 may be repeated for the second free end 18a of the first TFM 18, with the end result being that the second free end 18a is wedged into a second provisional locking cleat 732 under tension, as shown by way of example in FIG. 140. Once both the first and second free end 18a of the TFM 18 have been provisionally secured within the first and second cleats 732, the user (e.g., surgeon) may proceed with securing a second TFM 18 to the base 712.

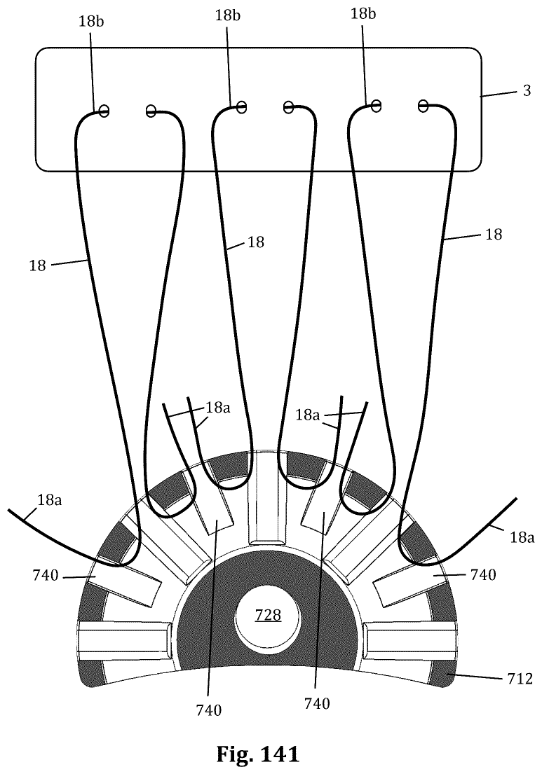

In some embodiments, a next step 940 in the method 930 is to engage a second portion of object 3 with an engagement portion 18b of a second tensionable fixation member (TFM) 18. By way of example, this may be done by passing the second TFM 18 through the second portion of object 3 or by wrapping the second TFM 18 around the object 3. In some embodiments, a next step 942 of the method 930 is to guide a first free end 18a of the second TFM 18 through a third provisional locking cleat 732 of the base 712, as shown by way of example in FIG. 141. In some embodiments, a next step 943 of the method 930 is to apply a desired tension to the second TFM 18. In some embodiments, a next step 944 of the method 930 is to provisionally secure the first free end 18a of the second TFM 18 to the base 712 under tension, for example by pulling on the first free end 18a to wedge the first free end 18a into the tapered wedge recess 736 of the third cleat 732, as described above. In some embodiments, these steps 940-944 may be repeated for the second free end 18a of the second TFM 18, with the end result being that the second free end 18a is wedged into a fourth provisional locking cleat 732 under tension, as shown by way of example in FIG. 141. In some embodiments, steps 934-944 may be repeated as many times as needed to secure the desired number of TFMs 18 to the base 712.

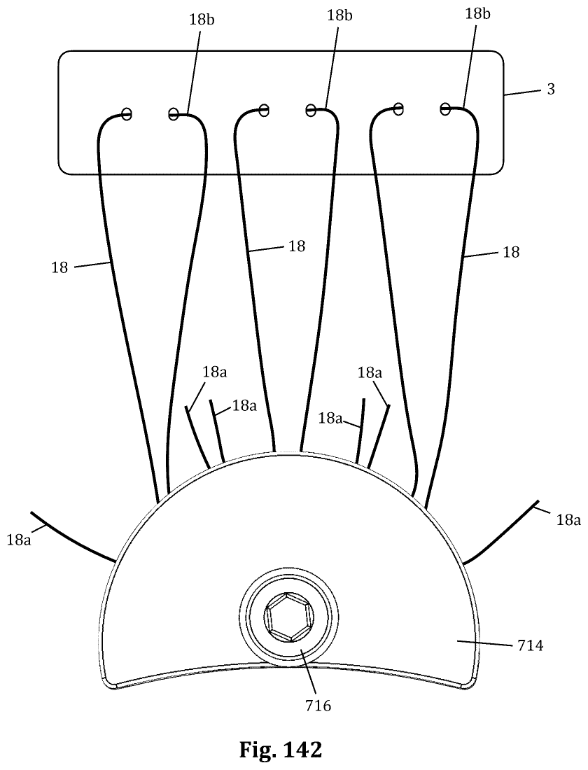

Once all desired TFMs 18 have been secured to their target objects and to the base 712, a next step 946 in the method 930 is to attach the locking element 714 to the base 712 such that the first and second (and third, fourth, etc.) TFMs 18 are captured within the locking interface between the locking element 714 and the base 712, as described above. In some embodiments, a next step 948 in the method 930 is to secure the locking element 714 to the base under compression with the lock screw 716, as shown by way of example in FIG. 142. At this point, the excess tensionable fixation member 18 material may be cut off, tied into a knot (for additional but unnecessary security) or simply left alone.

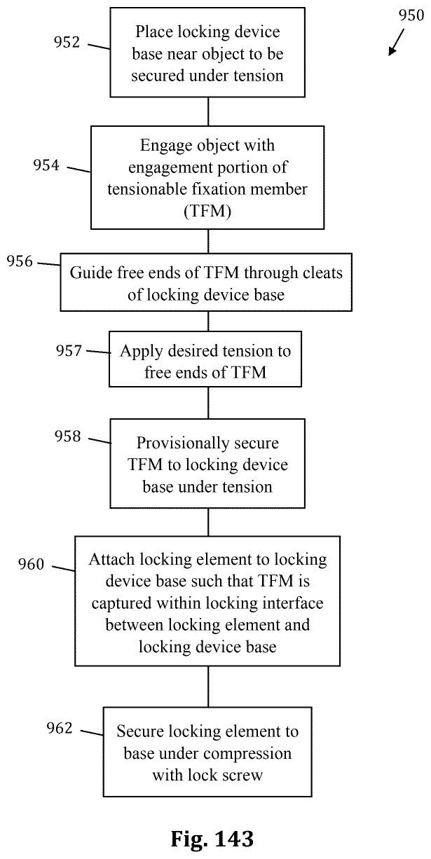

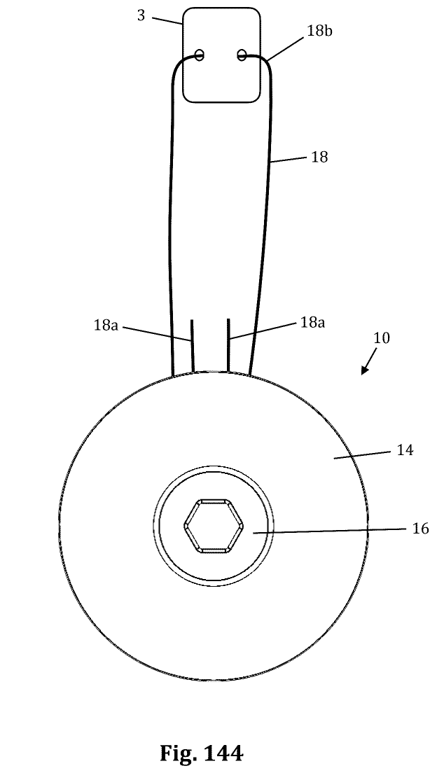

Referring to FIGS. 143-144, in some embodiments, any of the multi-strand knotless tensionable fixation systems described herein may be used in a method 950 of securing an object 3 under tension with at least one tensionable fixation member. In some embodiments, a first step 952 in the method 950 is to Referring to FIGS. 143-144, in some embodiments, any of the multi-strand knotless tensionable fixation systems described herein may be used in a method 950 of securing at least an object under tension with at least one tensionable fixation member. In some embodiments, the method 950 may be used in orthopedic fracture fixation procedures, in which case the object to be secured under tension may comprise one or more of bone (e.g., fractured sections of bone, tuberosities), muscle, tendon, and/or surgical implants. By way of example the method 950 is described herein using the system 10, described above with reference to FIGS. 1-14, however this method can be following using any of the multi-strand knotless tensionable fixation systems 10, 110, 210, 410, 610, 710, 810 described herein. In some embodiments, a first step 952 in the method 950 is to place a base 12 of the multi-strand knotless tensionable fixation system 10 near the object 3 to be secured under tension. For example, in some embodiments the object 3 may represent a fractured bone or surgical prosthesis.

In some embodiments, a next step 954 of the method 950 is to engage the object 3 with an engagement portion 18b of a tensionable fixation member (TFM) 18. By way of example, this may be done by passing the TFM 18 through the object 3 or by wrapping the TFM 18 around the object 3. In some embodiments, a next step 956 of the method 950 is to guide a first free end 18a of the TFM 18 through a first provisional locking cleat 32 of the base 12. In some embodiments, a next step 957 of the method 950 is to apply a desired tension to the TFM 18. In some embodiments, a next step 958 of the method 950 is to provisionally secure the first free end 18a of the TFM 18 to the base 12 under tension, for example by pulling on the first free end 18a to wedge the first free end 18a into tapered end 36 of the cleat 32, as described above. In some embodiments, these steps 954-958 may be repeated for the second free end 18a of the TFM 18, with the end result being that the second free end 18a is wedged into a second provisional locking cleat 32 under tension. Once both the first and second free ends 18a of the TFM 18 have been provisionally secured within the first and second cleats 32, the user (e.g., surgeon) may proceed with securing a second TFM 18 to the base 12.

Once the TFM 18 has been secured to its target object and to the base 12, a next step 960 in the method 950 is to attach the locking element 14 to the base 12 such that the TFM 18 is captured within the locking interface between the locking element 14 and the base 12, as described above. In some embodiments, a next step 962 in the method 950 is to secure the locking element 14 to the base under compression with the lock screw 16, as shown by way of example in FIG. 144. At this point, the excess tensionable fixation member 18 material may be cut off, tied into a knot (for additional but unnecessary security) or simply left alone.

Optionally, any embodiment described herein can have one or multiple cleats in the base. In any embodiment described herein, the angle of the fixation channel can be variable and sometimes maybe designed specifically to work with a certain needle design. In any embodiment described herein, the fixation channel can have a variable diameter. In any embodiment described herein, the shape of the embodiment can be varied based on targeted anatomy and the components can have additional features to allow stabilization on the bone using guide pins or screws. In any embodiment described herein, the length of each of these components can be variable. In any embodiment described herein, the base may have one or more cleats with different dimensions and/or orientations based for use with different sutures, procedures, locations, etc. In any embodiment described herein, the secondary fixation between the locking element and base can be either a convex/concave mating surface interaction or can be two planer mating surfaces interacting. In any embodiment described herein, the base may include transverse channels extending therethrough. In any embodiment described herein, the system may include a secondary tunnel plate.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or system that “comprises,” “has,” “includes” or “contains” one or more steps or elements, possesses those one or more steps or elements, but is not limited to possessing only those one or more elements. Likewise, a step of a method or an element of a system that “comprises,” “has,” “includes” or “contains” one or more features, possesses those one or more features, but is not limited to possessing only those one or more features. Furthermore, a system or structure that is configured in a certain way is configured in at least that way but may also be configured in ways that are not listed.

All patents and publications mentioned in this specification are indicative of the levels of those skilled in the art to which the disclosure pertains. It is to be understood that while a certain form of the disclosure is illustrated, it is not to be limited to the specific form or arrangement herein described and shown. It will be apparent to those skilled in the art that various changes may be made without departing from the scope of the disclosure and the disclosure is not to be considered limited to what is shown and described in the specification and any drawings/figures included herein.

One skilled in the art will readily appreciate that the present disclosure is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the disclosure and are defined by the scope of the appended claims. Although the disclosure has been described in connection with specific preferred embodiments, it should be understood that the disclosure as claimed should not be unduly limited to such specific embodiments. Indeed, various modifications of the described modes for carrying out the disclosure which are obvious to those skilled in the art are intended to be within the scope of the following claims.

What is claimed is:

1. A multi-strand knotless tensionable fixation system, comprising:
a plurality of tensionable fixation members, each tensionable fixation member of the plurality of tensionable fixation members having at least one free end and a target engagement portion;
a base member having a proximal surface, a distal surface, a laterally facing perimeter surface, a central opening extending between the proximal and distal surface, a first locking interface feature positioned on the proximal surface, and a plurality of provisional locking cleats formed within the proximal surface;
a locking element having a proximal surface, a distal surface, a central opening extending between the proximal and distal surfaces, and a second locking interface feature positioned on the distal surface; and
a compression member configured to extend through the central opening of the locking element and into the central opening of the base member, the compression member having a radial flange configured to engage the proximal surface of the locking element;
wherein each provisional locking cleat of said plurality of provisional locking cleats is configured to hold fast an associated one of the plurality of tensionable fixation members extending between the provisional locking cleat and a target object under tension;
wherein the compression member is configured such that actuation of the compression member causes the compression member to translate distally within the central opening of the base member such that the radial flange exerts a compressive force on the locking element; and
wherein the first locking interface feature and second locking interface feature are configured to cooperate with one another to pinch said plurality of tensionable fixation members associated with said plurality of provisional locking cleats upon actuation of the compression member.

2. The system of claim 1, wherein the compression member is a lock screw having a head and a threaded shank, and the head comprises the radial flange.

3. The system of claim 1, wherein each provisional locking cleat of the plurality of provisional locking cleats comprises a tapered wedge member configured to engage one of said plurality of tensionable fixation members.

4. The system of claim 1, wherein the base member further comprises a plurality of lateral openings formed in said laterally facing surface, each one of said plurality of lateral openings providing access into a corresponding one of said plurality of provisional locking cleats.

5. The system of claim 4, wherein each one of the plurality of lateral openings is configured to enable passage of one of said plurality of tensionable fixation members therethrough.

6. The system of claim 1, wherein the base includes one or more transverse channels extending therethrough.

7. The system of claim 1, wherein the first locking interface feature is a raised portion of the proximal surface extending around the perimeter of the base member.

8. The system of claim 1, wherein the second locking interface features is a recess of the distal surface extending around the perimeter of the locking element.

9. The system of claim 1, further comprising a coupling member configured to couple the base member to at least one of a fixation plate, artificial prosthesis, and bone.

10. The system of claim 1, wherein the second locking interface comprises a distal facing protrusion extending from the distal surface of the locking element.

11. A method of securing one or more objects with a multi-strand tensionable fixation system, comprising:
positioning a multi-strand tensionable fixation system near one or more target objects to be secured under tension, the multi-strand tensionable fixation system comprising:
a plurality of tensionable fixation members, each tensionable fixation member of the plurality of tensionable fixation members having at least one free end and a target engagement portion;
a base member having a proximal surface, a distal surface, a laterally facing perimeter surface, a central opening extending between the proximal and distal surface, a first locking interface feature positioned on the proximal surface, and a plurality of provisional locking cleats formed within the proximal surface, each provisional locking cleat of said plurality of provisional locking cleats configured to hold fast an associated one of said plurality of tensionable fixation members extending between the provisional locking cleat and a target object under tension;
a locking element having a proximal surface, a distal surface, a central opening extending between the proximal and distal surfaces, and a second locking interface feature positioned on the distal surface;
a compression member a compression member configured to extend through the central opening of the locking element and into the central opening of the base member, the compression member having a radial flange configured to engage the proximal surface of the locking element;
engaging a first target object of said one or more target objects with a target engagement portion of a first tensionable fixation member of said plurality of tensionable fixation members;
provisionally securing a first free end of said first tensionable fixation member within a first provisional locking cleat of said plurality of provisional locking cleats;
provisionally securing a second free end of said first tensionable fixation member within a second provisional locking cleat of said plurality of provisional locking cleats;
engaging a second target object of said one or more target objects with a target engagement portion of a second tensionable fixation member of said plurality of tensionable fixation members;
provisionally securing a first free end of said second tensionable fixation member within a third provisional locking cleat of said plurality of provisional locking cleats;
provisionally securing a second free end of said second tensionable fixation member within a fourth provisional locking cleat of said plurality of provisional locking cleats;
positioning the locking element on the base member such that the provisionally secured first and second free ends of said first tensionable fixation member and first and second free ends of said second tensionable fixation member are positioned between the first locking interface feature of the base member and the second locking interface feature of the locking element; and
advancing the compression member through the central opening of the locking element and into the central opening of the base member such that the radial flange contacts the proximal surface of the locking element; and
actuating the compression member such that the compression member translates distally within the central opening of the base member and the radial flange exerts a compressive force on the engaged proximal surface of the locking element, thereby causing the provisionally secured first and second free ends of said first tensionable fixation member and first and second free ends of said second tensionable fixation member to be pinched between the first locking interface feature of the base member and the second locking interface feature of the locking element to achieve a final locked state.

12. The method of claim 11, wherein the step of provisionally securing a first free end of said first tensionable fixation member within a first provisional locking cleat of said plurality of provisional locking cleats further comprises the sub-steps of:
passing the first free end of said first tensionable fixation member into the first provisional locking cleat of said plurality of provisional locking cleats;
applying a desired amount of tension to the first free end of said first tensionable fixation member; and
pulling on said free end to wedge said first free end of said first tensionable fixation member into a tapered wedge portion of said first provisional locking cleat under said desired amount of tension.

13. The method of claim 11, wherein the step of provisionally securing a second free end of said first tensionable fixation member within a second provisional locking cleat of said plurality of provisional locking cleats further comprises the sub-steps of:
passing the second free end of said first tensionable fixation member into the second provisional locking cleat of said plurality of provisional locking cleats;
applying a desired amount of tension to the second free end of said first tensionable fixation member; and
pulling on said second free end to wedge said second free end of said first tensionable fixation member into a tapered wedge portion of said second provisional locking cleat under said desired amount of tension.

14. The method of claim 11, wherein the step of provisionally securing a first free end of said second tensionable fixation member within a third provisional locking cleat of said plurality of provisional locking cleats further comprises the sub-steps of:
passing the second free end of said first tensionable fixation member into the third provisional locking cleat of said plurality of provisional locking cleats;
applying a desired amount of tension to the first free end of said second tensionable fixation member; and
pulling on said first free end of said second tensionable fixation member to wedge said first free end of said second tensionable fixation member into a tapered wedge portion of said third provisional locking cleat under said desired amount of tension.

15. The method of claim 11, wherein the step of provisionally securing a second free end of said second tensionable fixation member within a fourth provisional locking cleat of said plurality of provisional locking cleats further comprises the sub-steps of:
passing the second free end of said second tensionable fixation member into the fourth provisional locking cleat of said plurality of provisional locking cleats;
applying a desired amount of tension to the second free end of said second tensionable fixation member; and
pulling on said second free end of said second tensionable fixation member to wedge said second free end of said second tensionable fixation member into a tapered wedge portion of said fourth provisional locking cleat under said desired amount of tension.

16. The method of claim 11, wherein said first and second provisional locking cleats are positioned adjacent to one another.

17. The method of claim 11, wherein the third and fourth provisional locking cleats are positioned adjacent to one another.

18. The method of claim 11, further comprising the step of:
coupling the base member to an orthopedic fixation plate, artificial prosthesis, or bone segment.

19. The method of claim 11, wherein the compression member is a lock screw having a head and a threaded shank, and the head comprises the radial flange.

20. The method of claim 11, wherein each provisional locking cleat of the plurality of provisional locking cleats comprises a tapered wedge member configured to engage one of said plurality of tensionable fixation members.
